SEQUENCE LISTING (48 sequences)

SEQ ID NO: 1 - protein, 193 aa, Homo sapiens
MSGPPSAPQGALAAPRSPAVRRKGLQAPSWGSPGRPAAHSPWACGPPHWGPQRGPRNAAAARPGPRSRWHKRCAAACGACARPPGHQLQPPGAGAPQPGVACSYLGPRPQRTPCSAQSRPGWCAGPRRRHAPGTEPHVAPGRAPPPRAGASPGSRLLPGSPSLLLPAATWRTWGQESKVLRKILKAWDPFSLL

SEQ ID NO: 2 - protein, 2472 aa, Homo sapiens
MDPSGVKVLETAEDIQERRQQVLDRYHRFKELSTLRRQKLEDSYRFQFFQRDAEELEKWIQEKLQIASDENYKDPTNLQGKLQKHQAFEAEVQANSGAIVKLDETGNLMISEGHFASETIRTRLMELHRQWELLLEKMREKGIKLLQAQKLVQYLRECEDVMDWINDKEAIVTSEELGQDLEHVEVLQKKFEEFQTDMAAHEERVNEVNQFAAKLIQEQHPEEELIKTKQDEVNAAWQRLKGLALQRQGKLFGAAEVQRFNRDVDETISWIKEKEQLMASDDFGRDLASVQALLRKHEGLERDLAALEDKVKALCAEADRLQQSHPLSATQIQVKREELITNWEQIRTLAAERHARLNDSYRLQRFLADFRDLTSWVTEMKALINADELASDVAGAEALLDRHQEHKGEIDAHEDSFKSADESGQALLAAGHYASDEVREKLTVLSEERAALLELWELRRQQYEQCMDLQLFYRDTEQVDNWMSKQEAFLLNEDLGDSLDSVEALLKKHEDFEKSLSAQEEKITALDEFATKLIQNNHYAMEDVATRRDALLSRRNALHERAMRRRAQLADSFHLQQFFRDSDELKSWVNEKMKTATDEAYKDPSNLQGKVQKHQAFEAELSANQSRIDALEKAGQKLIDVNHYAKDEVAARMNEVISLWKKLLEATELKGIKLREANQQQQFNRNVEDIELWLYEVEGHLASDDYGKDLTNVQNLQKKHALLEADVAAHQDRIDGITIQARQFQDAGHFDAENIKKKQEALVARYEALKEPMVARKQKLADSLRLQQLFRDVEDEETWIREKEPIAASTNRGKDLIGVQNLLKKHQALQAEIAGHEPRIKAVTQKGNAMVEEGHFAAEDVKAKLHELNQKWEALKAKASQRRQDLEDSLQAQQYFADANEAESWMREKEPIVGSTDYGKDEDSAEALLKKHEALMSDLSAYGSSIQALREQAQSCRQQVAPTDDETGKELVLALYDYQEKSPREVTMKKGDILTLLNSTNKDWWKVEVNDRQGFVPAAYVKKLDPAQSASRENLLEEQGSIALRQEQIDNQTRITKEAGSVSLRMKQVEELYHSLLELGEKRKGMLEKSCKKFMLFREANELQQWINEKEAALTSEEVGADLEQVEVLQKKFDDFQKDLKANESRLKDINKVAEDLESEGLMAEEVQAVQQQEVYGMMPRDETDSKTASPWKSARLMVHTVATFNSIKELNERWRSLQQLAEERSQLLGSAHEVQRFHRDADETKEWIEEKNQALNTDNYGHDLASVQALQRKHEGFERDLAALGDKVNSLGETAERLIQSHPESAEDLQEKCTELNQAWSSLGKRADQRKAKLGDSHDLQRFLSDFRDLMSWINGIRGLVSSDELAKDVTGAEALLERHQEHRTEIDARAGTFQAFEQFGQQLLAHGHYASPEIKQKLDILDQERADLEKAWVQRRMMLDQCLELQLFHRDCEQAENWMAAREAFLNTEDKGDSLDSVEALIKKHEDFDKAINVQEEKIAALQAFADQLIAAGHYAKGDISSRRNEVLDRWRRLKAQMIEKRSKLGESQTLQQFSRDVDEIEAWISEKLQTASDESYKDPTNIQSKHQKHQAFEAELHANADRIRGVIDMGNSLIERGACAGSEDAVKARLAALADQWQFLVQKSAEKSQKLKEANKQQNFNTGIKDFDFWLSEVEALLASEDYGKDLASVNNLLKKHQLLEADISAHEDRLKDLNSQADSLMTSSAFDTSQVKDKRDTINGRFQKIKSMAASRRAKLNESHRLHQFFRDMDDEESWIKEKKLLVGSEDYGRDLTGVQNLRKKHKRLEAELAAHEPAIQGVLDTGKKLSDDNTIGKEEIQQRLAQFVEHWKELKQLAAARGQRLEESLEYQQFVANVEEEEAWINEKMTLVASEDYGDTLAAIQGLLKKHEAFETDFTVHKDRVNDVCTNGQDLIKKNNHHEENISSKMKGLNGKVSDLEKAAAQRKAKLDENSAFLQFNWKADVVESWIGEKENSLKTDDYGRDLSSVQTLLTKQETFDAGLQAFQQEGIANITALKDQLLAAKHVQSKAIEARHASLMKRWSQLLANSAARKKKLLEAQSHFRKVEDLFLTFAKKASAFNSWFENAEEDLTDPVRCNSLEEIKALREAHDAFRSSLSSAQADFNQLAELDRQIKSFRVASNPYTWFTMEALEETWRNLQKIIKERELELQKEQRRQEENDKLRQEFAQHANAFHQWIQETRTYLLDGSCMVEESGTLESQLEATKRKHQEIRAMRSQLKKIEDLGAAMEEALILDNKYTEHSTVGLAQQWDQLDQLGMRMQHNLEQQIQARNTTGVTEEALKEFSMMFKHFDKDKSGRLNHQEFKSCLRSLGYDLPMVEEGEPDPEFEAILDTVDPNRDGHVSLQEYMAFMISRETENVKSSEEIESAFRALSSEGKPYVTKEELYQNLTREQADYCVSHMKPYVDGKGRELPTAFDYVEFTRSLFVN

SEQ ID NO: 3 - protein, 109 aa, Homo sapiens
MSARGEGAGQPSTSAQGQPAAPAPQKRGRGRPRKQQQEPTGEPSPKRPRGRPKGSKNKSPSKAAQKKAEATGEKRPRGRPRKWPQQVVQKKPAQEETEETSSQESAEED

SEQ ID NO: 4 - protein, 213 aa, Homo sapiens [sequence omitted]

SEQ ID NO: 5 - protein, 494 aa, Homo sapiens
MPRVYIGRLSYQARERDVERFFKGYGKILEVDLKNGYGFVEFDDLRDADDAVYELNGKDLCGERVIVEHARGPRRDGSYGSGRSGYGYRRSGRDKYGPPTRTEYRLIVENLSSRCSWQDLKDYMRQAGEVTYADAHKGRKNEGVIEFVSYSDMKRALEKLDGTEVNGRKIRLVEDKPGSRRRRSYSRSRSHSRSRSRSRHSRKSRSRSGSSKSSHSKSRSRSRSGSRSRSKSRSRSQSRSRSKKEKSRSPSKEKSRSRSHSAGKSRSKSKDQAEEKIQNNDNVGKPKSRSPSRHKSKSKSRSRSQERRVEEEKRGSVSRGRSQEKSLRQSRSRSRSKGGSRSRSRSRSKSKDKRKGRKRSREESRSRSRSRSKSERSRKRGSKRDSKAGSSKKKKKEDTDRSQSRSPSRSVSKEREHAKSESSQREGRGESENAGTNQETRSRSRSNSKSKPNLPSESRSRSKSASKTRSRSKSRSRSASRSPSRSRSRSHSRS

SEQ ID NO: 6 - protein, 271 aa, Homo sapiens
MEAAAEPGNLAGVRHIILVLSGKGGVGKSTISTELALALRHAGKKVGILDVDLCGPSIPRMLGAQGRAVHQCDRGWAPVFLDREQSISLMSVGFLLEKPDEAVVWRGPKKNALIKQFVSDVAWGELDYLVVDTPPGTSDEHMATIEALRPYQPLGALVVTTPQAVSVGDVRRELTFCRKTGLRVMGIVENMSGFTCPHCTECTSVFSRGGGEELAQLAGVPFLGSVPLDPALMRTLEEGHDFIQEFPGSPAFAALTSIAQKILDATPACLP

SEQ ID NO: 7 - protein, 268 aa, Homo sapiens
MAAVRALVASRLAAASAFTSLSPGGRTPSQRAALHLSVPRPAARVALVLSGCGVYDGTEIHEASAILVHLSRGGAEVQIFAPDVPQMHVIDHTKGQPSEGESRNVLTESARIARGKITDLANLSAANHDAAIFPGGFGAAKNLSTFAVDGKDCKVNKEVERVLKEFHQAGKPIGLCCIAPVLAAKVLRGVEVTVGHEQEEGGKWPYAGTAEAIKALGAKHCVKEVVEAHVDQKNKVVTTPAFMCETALHYIHDGIGAMVRKVLELTGK

SEQ ID NO: 8 - protein, 372 aa, Homo sapiens
MSGRSGKKKMSKLSRSARAGVIFPVGRLMRYLKKGTFKYRISVGAPVYMAAVIEYLAAEILELAGNAARDNKKARIAPRHILLAVANDEELNQLLKGVTIASGGVLPRIHPELLAKKRGTKGKSETILSPPPEKRGRKATSGKKGGKKSKAAKPRTSKKSKPKDSDKEGTSNSTSEDGPGDGFTILSSKSLVLGQKLSLTQSDISHIGSMRVEGIVHPTTAEIDLKEDIGKALEKAGGKEFLETVKELRKSQGPLEVAEAAVSQSSGLAAKFVIHCHIPQWGSDKCEEQLEETIKNCLSAAEDKKLKSVAFPPFPSGRNCFPKQTAAQVTLKAISAHFDDSSASSLKNVYFLLFDSESIGIYVQEMAKLDAK

SEQ ID NO: 9 - protein, 606 aa, Homo sapiens
MAVTFEDVTIIFTWEEWKFLDSSQKRLYREVMWENYTNVMSVENWNESYKSQEEKFRYLEYENFSYWQGWWNAGAQMYENQNYGETVQGTDSKDLTQQDRSQCQEWLILSTQVPGYGNYELTFESKSLRNLKYKNFMPWQSLETKTTQDYGREIYMSGSHGFQGGRYRLGISRKNLSMEKEQKLIVQHSYIPVEEALPQYVGVICQEDLLRDSMEEKYCGCNKCKGIYYWNSRCVFHKRNQPGENLCQCSICKACFSQRSDLYRHPRNHIGKKLYGCDEVDGNFHQSSGVHFHQRVHIGEVPYSCNACGKSFSQISSLHNHQRVHTEEKFYKIECDKDLSRNSLLHIHQRLHIGEKPFKCNQCGKSFNRSSVLHVHQRVHTGEKPYKCDECGKGFSQSSNLRIHQLVHTGEKSYKCEDCGKGFTQRSNLQIHQRVHTGEKPYKCDDCGKDFSHSSDLRIHQRVHTGEKPYTCPECGKGFSKSSKLHTHQRVHTGEKPYKCEECGKGFSQRSHLLIHQRVHTGEKPYKCHDCGKGFSHSSNLHIHQRVHTGEKPYQCAKCGKGFSHSSALRIHQRVHAGEKPYKCREYYKGFDHNSHLHNNHRRGNL

SEQ ID NO: 10 - protein, 1188 aa, Homo sapiens
MKKKQTVQGTFSKLFGKKHTTTPSTSLYATNPPWIFTQEAPEEGTGGFDGIYYGDNRFNTVSESGTATLKARPRVRPLLTFLPLNAQENHGLAVPTPSVPDDFADKEVTGTSSLVNGNLRLYSSVGDLRPGQYGQDLLIPPPPPGPAPGPPQDISEPPGGSPLPSPPSTAPPPPPLLLEPPPPPSMAPPPPPVLEALSPPHTLSSPSIPTPPDFIPPAPPLAFLAPPPPPVPAPAPPAPASPHTVGTRLFPPGGVTKWKSDVALNGRQAEATRASPPRSPAEPKGSALGPNPEPHLTFPRSFKVPPPTPVRTSSIPVQEAQEAPRKEEGATKKAPSRLPLPPSFHIRPASQVYPDRAPEPDCPGELKATAPASPRLGQSQSQADERAGTPPPAPPLPPPAPPLPPPAPPLPPAAPPLPCAQKAAHPPAGFTKTPKSSSPALKPKPNPPSPENTASSAPVDWRDPSQMEKLRNELAAYLCGSRREDRFLSHRPGPTVAPQSKEGKKGPRLPEKETLLSLPAKDTPPGVPEKSLGGSSLTETEAAPSLTLPSVDYIPQDSPTPSVRQIRNELEARLSSAAEKEAKPSIGSLPPKPRLEGGRICENGADDDKLSKPVAKNLPPQSTTLLPTTSLQPKAMLGPAIPPKATPEPAIPPKATLWPATPPKATLGPATPLKATSGPTTPLKATSGPAIASTATTLPTTTSQLMAEKDSGPAGQPEKPASQEVSTPSQARGEGSPSEATRLPTQGARSSAAFPPKTSPGGGEVPCLYKPHCHQSSLSREVAVVMPTLARGGAAGPGEPVEVKEPPGLPAKPPASAQPTDELLRHPVTGEVVERGSPMALLLAARQRAQKGRSVGAALGRSSLPGSLRDHSHQAEASSDSIFHSQGTPNSFTVVPKLPKEAEKDSPLTTEIPNKWGPRLGRDAEGTELSRRHNWTKPEPQAPVAWERVAPSNLPQGHPLPKSFSSPPSPSNKREEEEEEFNFEVIPPPPEFSNDPEPPAPALQYLGRQSSPPRNNYSDLRQLPNAGPGAPPALGFSRFPAGARYAGAGGLERFSGGGRSLIKKRLYVGEPHRGPGLPHGGTGRSLSSPNCFGPQPGGPEMRRVNSAGRAPPGGLHAPRLSLEGAARGAAEAKHKAPGSADYGFAPAAGRSPYTTTRYGSPINTFTVRPGTRHPISYVCSGAHRKATS

SEQ ID NO: 11 - protein, 869 aa, Homo sapiens
MEPETALWGPDLQGPEQSPNDAHRGAESENEEESPRQESSGEEIIMGDPAQSPESKDSTEMSLERSSQDPSVPQNPPTPLGHSNPLDHQIPLDPPAPEVVPTPSDWTKACEASWQWGALTTWNSPPVVPANEPSLRELVQGRPAGAEKPYICNECGKSFSQWSKLLRHQRIHTGERPNTCSECGKSFTQSSHLVQHQRTHTGEKPYKCPDCGKCFSWSSNLVQHQRTHTGEKPYKCTECEKAFTQSTNLIKHQRSHTGEKPYKCGECRRAFYRSSDLIQHQATHTGEKPYKCPECGKRFGQNHNLLKHQKIHAGEKPYRCTECGKSFIQSSELTQHQRTHTGEKPYECLECGKSFGHSSTLIKHQRTHLREDPFKCPVCGKTFTLSATLLRHQRTHTGERPYKCPECGKSFSVSSNLINHQRIHRGERPYICADCGKSFIMSSTLIRHQRIHTGEKPYKCSDCGKSFIRSSHLIQHRRTHTGEKPYKCPECGKSFSQSSNLITHVRTHMDENLFVCSDCGKAFLEAHELEQHRVIHERGKTPARRAQGDSLLGLGDPSLLTPPPGAKPHKCLVCGKGFNDEGIFMQHQRIHIGENPYKNADGLIAHAAPKPPQLRSPRLPFRGNSYPGAAEGRAEAPGQPLKPPEGQEGFSQRRGLLSSKTYICSHCGESFLDRSVLLQHQLTHGNEKPFLFPDYRIGLGEGAGPSPFLSGKPFKCPECKQSFGLSSELLLHQKVHAGGKSSQKSPELGKSSSVLLEHLRSPLGARPYRCSDCRASFLDRVALTRHQETHTQEKPPNPEDPPPEAVTLSTDQEGEGETPTPTESSSHGEGQNPKTLVEEKPYLCPECGAGFTEVAALLLHRSCHPGVSL

SEQ ID NO: 12 - protein, 623 aa, Homo sapiens
MAGFSHFSQPPYRDLWEPPRPGGERESTQRLGGQRSGADSTACSRAGTPGAESEAGACWLHPHCSFTPRPRRRGCSDSLRGSRSLSDVARRPLERSRKHRPRSRRLEDAWGETGTKPRPAWQPQTQLPPQRPQPCPHYPLAQGDSPPPCPGGAGTPLSGTFRVEKAQGGDQWAVPLGRHLGRWSPSSVPSERSSVPSQKFKRHSACVCAQKRDSSDQVESLASRDSQPLASSKEMRSPHTQVLKSKLEEVVVSSQDQQIVALVLTRLKKAQRIRELQQQAAKAWEELKRSDQKVQMTLERERRLLLRQSQEQWQEKEQRKTLQSPEQRGLRRDSQRKNVPPGESRWKEQPEDQESPRQEKLEKARAQAEHRKQCQVRRLREQEKMLRNLREQHSLQLQRRLVEACRKRHLHAVEGQKKVQDTNLSSLINYQARKVLMDCQAKAEELLRQLSLEQSFQRSQEIHQGLRKERQRELREKAQKEEEQLQQARWRAGESEEQRKMRKRILVELADEKIRQARSHVHKTTRDKVQHLRELNHLREKNHHILKLKAEKEEKCHIEGIKEAIKKKEQRVQHISQGKDPNFQEFQKLPQASRREERAPPNSSLDQMVLEAQLRACQQNRGY

SEQ ID NO: 13 - protein, 660 aa, Homo sapiens
MATSHRVAKLVASSLQTPVNPITGARVAQYEREDPLKALAAAEAILEDEEEEKVAQPAGASADLNTSFSGVDEHAPISYEDFVNFPDIHHSNEEYFKKVEELKAAHIETMAKLEKMYQDKLHLKEVQPVVIREDSLSDSSRSVSEKNSYHPVSLMTSFSEPDLGQSSSLYVSSSEEELPNLEKEYPRKNRMMTYAKELINNMWTDFCVEDYIRCKDTGFHAAEKRRKKRKEWVPTITVPEPFQMMIREQKKKEESMKSKSDIEMVHKALKKQEEDPEYKKKFRANPVPASVFLPLYHDLVKQKEERRRSLKEKSKEALLASQKPFKFIAREEQKRAAREKQLRDFLKYKKKTNRFKARPIPRSTYGSTTNDKLKEEELYRNLRTQLRAQEHLQNSSPLPCRSACGCRNPRCPEQAVKLKCKHKVRCPTPDFEDLPERYQKHLSEHKSPKLLTVCKPFDLHASPHASIKREKILADIEADEENLKETRWPYLSPRRKSPVRCAGVNPVPCNCNPPVPTVSSRGREQAVRKSEKERMREYQRELEEREEKLKKRPLLFERVAQKNARMAAEKHYSNTLKALGISDEFVSKKGQSGKVLEYFNNQETKSVTEDKESFNEEEKIEERENGEENYFIDTNSQDSYKEKDEANEESEEEKSVEESH

SEQ ID NO: 14 - protein, 477 aa, Homo sapiens
MLRWLRDFVLPTAACQDAEQPTRYETLFQALDRNGDGVVDIGELQEGLRNLGIPLGQDAEEKIFTTGDVNKDGKLDFEEFMKYLKDHEKKMKLAFKSLDKNNDGKIEASEIVQSLQTLGLTISEQQAELILQSIDVDGTMTVDWNEWRDYFLFNPVTDIEEIIRFWKHSTGIDIGDSLTIPDEFTEDEKKSGQWWRQLLAGGIAGAVSRTSTAPLDRLKIMMQVHGSKSDKMNIFGGFRQMVKEGGIRSLWRGNGTNVIKIAPETAVKFWAYEQYKKLLTEEGQKIGTFERFISGSMAGATAQTFIYPMEVMKTRLAVGKTGQYSGIYDCAKKILKHEGLGAFYKGYVPNLLGIIPYAGIDLAVYELLKSYWLDNFAKDSVNPGVMVLLGCGALSSTCGQLASYPLALVRTRMQAQAMLEGSPQLNMVGLFRRIISKEGIPGLYRGITPNFMKVLPAVGISYVVYENMKQTLGVTQK

SEQ ID NO: 15 - protein, 913 aa, Homo sapiens
MTKFQEMVTFKDVAVVFTEEELGLLDSVQRKLYRDVMLENFRNLLLVAHQPFKPDLISQLEREEKLLMVETETPRDGCSGRKNQQKMESIQEVTVSYFSPKELSSRQTWQQSAGGLIRCQDFLKVFQGKNSQLQEQGNSLGQVWAGIPVQISEDKNYIFTHIGNGSNYIKSQGYPSWRAHHSWRKMYLKESHNYQCRCQQISMKNHFCKCDSVSWLSHHNDKLEVHRKENYSCHDCGEDIMKVSLLNQESIQTEEKPYPCTGYRKAFSNDSSSEVHQQFHLEGKPYTYSSCGKGCNYSSLLHIHQNIEREDDIENSHLKSYQRVHTEEKPCKCGEYGENFNHCSPLNTYELIHTGEMSYRHNIYEKAFSHSLDLNSIFRVHTRDEPHEYEENENVFNQSSCLQVHQKIHTEEKLYTDIEYGKSFICSSNLDIQHRVHMEENSYNSEECGNGFSLASHFQDLQIVHTKEQPYKRYVCSNSFSHNLYLQGHPKIHIGEKPRKEHGNGFNWSSKLKDHQRVHTGQKPYKCNICGKGFNHRSVLNVHQRVHTGEKPYKCEECDKGFSRSSYLQAHQRVHTGEKPYKCEECGKGFSRNSYLQGHQRVHTGEKPYKCEECGKGFSRSSHLQGHQRVHTGEKPFKCEECGKGFSWSFNLQIHQRVHTGEKPYKCEECGKGFSKASTLLAHQRVHTGEKPYQCDECGKSFSQRSYLQSHQSVHSGERPYICEVCGKGFSQRAYLQGHQRVHTRVKPYKCEMCGKGFSQSSRLEAHRRVHTGGKPYKCEVCTKGFSESSRLQAHQRVHVEGRPYKCEQCGKGFSGYSSLQAHHRVHTGEKPYKCEVCGKGFSQRSNLQAHQRVHTGEKPYKCDACGKGFRWSSGLLIHQRVHSSDKFYKSEDYGKDYPSSENLHRNEDSVLF

SEQ ID NO: 16 - protein, 654 aa, Homo sapiens
MAFRDVAVDFTQDEWRLLSPAQRTLYREVMLENYSNLVSLGISFSKPELITQLEQGKETWREEKKCSPATCPADPEPELYLDPFCPPGFSSQKFPMQHVLCNHPPWIFTCLCAEGNIQPGDPGPGDQEKQQQASEGRPWSDQAEGPEGEGAMPLFGRTKKRTLGAFSRPPQRQPVSSRNGLRGVELEASPAQSGNPEETDKLLKRIEVLGFGTVNCGECGLSFSKMTNLLSHQRIHSGEKPYVCGVCEKGFSLKKSLARHQKAHSGEKPIVCRECGRGFNRKSTLIIHERTHSGEKPYMCSECGRGFSQKSNLIIHQRTHSGEKPYVCRECGKGFSQKSAVVRHQRTHLEEKTIVCSDCGLGFSDRSNLISHQRTHSGEKPYACKECGRCFRQRTTLVNHQRTHSKEKPYVCGVCGHSFSQNSTLISHRRTHTGEKPYVCGVCGRGFSLKSHLNRHQNIHSGEKPIVCKDCGRGFSQQSNLIRHQRTHSGEKPMVCGECGRGFSQKSNLVAHQRTHSGERPYVCRECGRGFSHQAGLIRHKRKHSREKPYMCRQCGLGFGNKSALITHKRAHSEEKPCVCRECGQGFLQKSHLTLHQMTHTGEKPYVCKTCGRGFSLKSHLSRHRKTTSVHHRLPVQPDPEPCAGQPSDSLYSL

SEQ ID NO: 17 - protein, 3088 aa, Homo sapiens
MEEFLQRAKSKLNRSKRLEKVHVVIGPKSCDLDSLISTFTYAYFLDKVSPPGVLCLPVLNIPRTEFNYFTETRFILEELNISESFHIFRDEINLHQLNDEGKLSITLVGSSVLASEDKTLESAVVKVINPVEQSDANVEFRESSSSLVLKEILQEAPELITEQLAHRLRGSILFKWMTMESEKISEKQEEILSILEEKFPNLPPREDIINVLQETQFSAQGLSIEQTMLKDLKELSDGEIKVAISTVSMNLENCLFHSNITSDLKAFTDKFGFDVLILFSSYLSEEQQPRRQIAVYSENMELCSQICCELEECQNPCLELEPFDCGCDEILVYQQEDPSVTCDQVVLVVKEVINRRCPEMVSNSRTSSTEAVAGSAPLSQGSSGIMELYGSDIEPQPSSVNFIENPPDLNDSNQAQVDANVDLVSPDSGLATIRSSRSSKESSVFLSDDSPVGEGAGPHHTLLPGLDSYSPIPEGAVAEEHAWSGEHGEHFDLFNFDPAPMASGQSQQSSHSADYSPADDFFPNSDLSEGQLPAGPEGLDGMGTNMSNYSSSSLLSGAGKDSLVEHDEEFVQRQDSPRDNSERNLSLTDFVGDESPSPERLKNTGKRIPPTPMNSLVESSPSTEEPASLYTEDMTQKATDTGHMGPPQTHARCSSWWGGLEIDSKNIADAWSSSEQESVFQSPESWKEHKPSSIDRRASDSVFQPKSLEFTKSGPWESEFGQPELGSNDIQDKNEESLPFQNLPMEKSPLPNTSPQGTNHLIEDFASLWHSGRSPTAMPEPWGNPTDDGEPAAVAPFPAWSAFGKEDHDEALKNTWNLHPTSSKTPSVRDPNEWAMAKSGFAFSSSELLDNSPSEINNEAAPEIWGKKNNDSRDHIFAPGNPSSDLDHTWTNSKPPKEDQNGLVDPKTRGKVYEKVDSWNLFEENMKKGGSDVLVPWEDSFLSYKCSDYSASNLGEDSVPSPLDTNYSTSDSYTSPTFAGDEKETEHKPFAKEEGFESKDGNSTAEETDIPPQSLQQSSRNRISSGPGNLDMWASPHTDNSSEINTTHNLDENELKTEHTDGKNISMEDDVGESSQSSYDDPSMMQLYNETNRQLTLLHSSTNSRQTAPDSLDLWNRVILEDTQSTATISDMDNDLDWDDCSGGAAIPSDGQTEGYMAEGSEPETRFTVRQLEPWGLEYQEANQVDWELPASDEHTKDSAPSEHHTLNEKSGQLIANSIWDSVMRDKDMSSFMLPGSSHITDSEQRELPPEIPSHSANVKDTHSPDAPAASGTSESEALISHLDKQDTERETLQSDAASLATRLENPGYFPHPDPWKGHGDGQSESEKEAQGATDRGHLDEEEVIASGVENASGISEKGQSDQELSSLVASEHQEICIKSGKISSLAVTFSPQTEEPEEVLEYEEGSYNLDSRDVQTGMSADNLQPKDTHEKHLMSQRNSGETTETSDGMNFTKYVSVPEKDLEKTEECNFLEPENVGGGPPHRVPRSLDFGDVPIDSDVHVSSTCSEITKNLDVKGSENSLPGAGSSGNFDRDTISSEYTHSSASSPELNDSSVALSSWGQQPSSGYQEENQGNWSEQNHQESELITTDGQVEIVTKVKDLEKNRINEFEKSFDRKTPTFLEIWNDSVDGDSFSSLSSPETGKYSEHSGTHQESNLIASYQEKNEHDISATVQPEDARVISTSSGSDDDSVGGEESIEEEIQVANCHVAEDESRAWDSLNESNKFLVTADPKSENIYDYLDSSEPAENENKSNPFCDNQQSSPDPWTFSPLTETEMQITAVEKEKRSSPETGTTGDVAWQISPKASFPKNEDNSQLEMLGFSADSTEWWKASPQEGRLIESPFERELSDSSGVLEINSSVHQNASPWGVPVQGDIEPVETHYTNPFSDNHQSPFLEGNGKNSHEQLWNIQPRQPDPDADKFSQLVKLDQIKEKDSREQTFVSAAGDELTPETPTQEQCQDTMLPVCDHPDTAFTHAEENSCVTSNVSTNEGQETNQWEQEKSYLGEMTNSSIATENFPAVSSPTQLIMKPGSEWDGSTPSEDSRGTFVPDILHGNFQEGGQLASAAPDLWIDAKKPFSLKADGENPDILTHCEHDSNSQASDSPDICHDSEAKQETEKHLSACMGPEVESSELCLTEPEIDEEPIYEPGREFVPSNAELDSENATVLPPIGYQADIKGSSQPASHKGSPEPSEINGDNSTGLQVSEKGASPDMAPILEPVDRRIPRIENVATSIFVTHQEPTPEGDGSWISDSFSPESQPGARALFDGDPHLSTENPALVPDALLASDTCLDISEAAFDHSFSDASGLNTSTGTIDDMSKLTLSEGHPETPVDGDLGKQDICSSEASWGDFEYDVMGQNIDEDLLREPEHFLYGGDPPLEEDSLKQSLAPYTPPFDLSYLTEPAQSAETIEEAGSPEDESLGCRAAEIVLSALPDRRSEGNQAETKNRLPGSQLAVLHIREDPESVYLPVGAGSNILSPSNVDWEVETDNSDLPAGGDIGPPNGASKEISELEEEKTIPTKEPEQIKSEYKEERCTEKNEDRHALHMDYILVNREENSHSKPETCEERESIAELELYVGSKETGLQGTQLASFPDTCQPASLNERKGLSAEKMSSKSDTRSSFESPAQDQSWMFLGHSEVGDPSLDARDSGPGWSGKTVEPFSELGLGEGPQLQILEEMKPLESLALEEASGPVSQSQKSKSRGRAGPDAVTLQAVTHDNEWEMLSPQPVQKNMIPDTEMEEETEFLELGTRISRPNGLLSEDVGMDIPFEEGVLSPSAADMRPEPPNSLDLNDTHPRRIKLTAPNINLSLDQSEGSILSDDNLDSPDEIDINVDELDTPDEADSFEYTGHDPTANKDSGQESESIPEYTAEEEREDNRLWRTVVIGEQEQRIDMKVIEPYRRVISHGGYYGDGLNAIIVFAACFLPDSSRADYHYVMENLFLYVISTLELMVAEDYMIVYLNGATPRRRMPGLGWMKKCYQMIDRRLRKNLKSFIIVHPSWFIRTILAVTRPFISSKFSSKIKYVNSLSELSGLIPMDCIHIPESIIKLDEELREASEAAKTSCLYNDPEMSSMEKDIDLKLKEKP

SEQ ID NO: 18 - protein, 597 aa, Homo sapiens
MSSTVNNGAASMQSTPDAANGFPQPSSSSGTWPRAEEELRAAEPGLVKRAHREILDHERKRRVELKCMELQEMMEEQGYSEEEIRQKVGTFRQMLMEKEGVLTREDRPGGHIVAETPRLTEGAEPGLEYAPFDDDDGPVDCDCPASCYRGHRGYRTKHWSSSSASPPPKKKKKKKGGHRRSRKKRRLESECSCGSSSPLRKKKKSVKKHRRDRSDSGSRRKRRHRSRSSKCKRKEKNKEKKRPHTESPGRRSHRHSSGSSHSPSLSSHYSDSRSPSRLSPKHRDEGRKTGSQRSSGSRSPSPSGGSGWGSPQRNGGSGQRSGAHGGRPGSAHSPPDKPSSPSPRVRDKAAAAAPTPPARGKESPSPRSAPSSQGRGGRAAGGAGRRRRRRRRRRRSRSSASAPRRRGRRRPRPAPPRGSSRSLSRARSSSDSGSGRGAPGPGPEPGSERGHGGHGKRAKERPPRARPASTSPSPGAHGRRGGPEGKSSSRSPGPHPRSWSSSRSPSKSRSRSAEKRPHSPSRSPSPKKPLSRDKDGEGRARHSEAEATRARRRSRSYSPIRKRRRDSPSFMEPRRITSAASVLFHTTGPAPLPPPAA

SEQ ID NO: 19 - protein, 669 aa, Homo sapiens
  MOD_RES 667: note = Selenocysteine
MAVYRAALGASLAAARLLPLGRCSPSPAPRSTLSGAAMEPAPRWLAGLRFDNRALRALPVEAPPPGPEGAPSAPRPVPGACFTRVQPTPLRQPRLVALSEPALALLGLGAPPAREAEAEAALFFSGNALLPGAEPAAHCYCGHQFGQFAGQLGDGAAMYLGEVCTATGERWELQLKGAGPTPFSRQADGRKVLRSSIREFLCSEAMFHLGVPTTRAGACVTSESTVVRDVFYDGNPKYEQCTVVLRVASTFIRFGSFEIFKSADEHTGRAGPSVGRNDIRVQLLDYVISSFYPEIQAAHASDSVQRNAAFFREVTRRTARMVAEWQCVGFCHGVLNTDNMSILGLTIDYGPFGFLDRYDPDHVCNASDNTGRYAYSKQPEVCRWNLRKLAEALQPELPLELGEAILAEEFDAEFQRHYLQKMRRKLGLVQVELEEDGALVSKLLETMHLTGADFTNTFYLLSSFPVELESPGLAEFLARLMEQCASLEELRLAFRPQMDPRQLSMMLMLAQSNPQLFALMGTRAGIARELERVEQQSRLEQLSAAELQSRNQGHWADWLQAYRARLDKDLEGAGDAAAWQAEHVRVMHANNPKYVLRNYIAQNAIEAAERGDFSEVRRVLKLLETPYHCEAGAATDAEATEADGADGRQRSYSSKPPLWAAELCVTXSS

SEQ ID NO: 20 - protein, 1300 aa, Homo sapiens
MEEASLCLGVSSAEPEAEPHLSGPVLNGQYAMSQKLHQITSQLSHAFPELHPRPNPEEKPPASLEEKAHVPMSGQPMGSQMALLANQLGREVDTSLNGRVDLQQFLNGQNLGIMSQMSDIEDDARKNRKYPCPLCGKRFRFNSILSLHMRTHTGEKPFKCPYCDHRAAQKGNLKIHLRTHKLGNLGKGRGRVREENRLLHELEERAILRDKQLKGSLLQPRPDLKPPPHAQQAPLAACTLALQANHSVPDVAHPVPSPKPASVQEDAVAPAAGFRCTFCKGKFKKREELDRHIRILHKPYKCTLCDFAASQEEELISHVEKAHITAESAQGQGPNGGGEQSANEFRCEVCGQVFSQAWFLKGHMRKHKDSFEHCCQICGRRFKEPWFLKNHMKVHLNKLSVKNKSPSDPEVPVPMGGMSQEAHANLYSRYLSCLQSGFMTPDKAGLSEPSQLYGKGELPMKEKEALGKLLSPISSMAHGVPEGDKHSLLGCLNLVPPLKSSCIERLQAAAKAAEMDPVNSYQAWQLMARGMAMEHGFLSKEHPLQRNHEDTLANAGVLFDKEKREYVLVGADGSKQKMPADLVHSTKVGSQRDLPSKLDPLESSRDFLSHGLNQTLEYNLQGPGNMKEKPTECPDCGRVFRTYHQVVVHSRVHKRDRKGEEDGLHVGLDERRGSGSDQESQSVSRSTTPGSSNVTEESGVGGGLSQTGSAQEDSPHPSSPSSSDIGEEAGRSAGVQQPALLRDRSLGSAMKDCPYCGKTFRTSHHLKVHLRIHTGEKPYKCPHCDYAGTQSASLKYHLERHHRERQNGAGPLSGQPPNQDHKDEMSSKASLFIRPDILRGAFKGLPGIDFRGGPASQQWTSGVLSSGDHSGQATGMSSEVPSDALKGTDLPSKSTHFSEIGRAYQSIVSNGVNFQGSLQAFMDSFVLSSLKKEKDMKDKALADPPSMKVHGVDGGEEKPSGKSSQRKSEKSQYEPLDLSVRPDAASLPGSSVTVQDSIAWHGCLFCAFTTSSMELMALHLQANHLGKAKRKDNTIGVTVNCKDQAREASKMALLPSLQSNKDLGLSNMISSLDSASEKMAQGQLKETLGEQKSGAWTGHVDPAFCNFPSDFYKQFGVYPGMVGSGASSSCPNKEPDGKAHSEEDVPILIPETTSKNTTDDLSDIASSEDMDSSKGENNDEEDVETEPEMMTKPLSALSKDSSSDGGDSLQPTGTSQPVQGLVSPLSQAPEKQWHSQGLLQAQDPLAGLPKPERGPQSLDKPMNMLSVLRAYSSDGLAAFNGLASSTANSGCIKRPDLCGK

SEQ ID NO: 21 - protein, 1623 aa, Homo sapiens
MAAEASKTGPSRSSYQRMGRKSQPWGAAEIQCTRCGRRVSRSSGHHCELQCGHAFCELCLLMTEECTTIICPDCEVATAVNTRQRYYPMAGYIKEDSIMEKLQPKTIKNCSQDFKKTADQLTTGLERSASTDKTLLNSSAVMLDTNTAEEIDEALNTAHHSFEQLSIAGKALEHMQKQTIEERERVIEVVEKQFDQLLAFFDSRKKNLCEEFARTTDDYLSNLIKAKSYIEEKKNNLNAAMNIARALQLSPSLRTYCDLNQIIRTLQLTSDSELAQVSSPQLRNPPRLSVNCSEIICMFNNMGKIEFRDSTKCYPQENEIRQNVQKKYNNKKELSCYDTYPPLEKKKVDMSVLTSEAPPPPLQPETNDVHLEAKNFQPQKDVATASPKTIAVLPQMGSSPDVIIEEIIEDNVESSAELVFVSHVIDPCHFYIRKYSQIKDAKVLEKKVNEFCNRSSHLDPSDILELGARIFVSSIKNGMWCRGTITELIPIEGRNTRKPCSPTRLFVHEVALIQIFMVDFGNSEVLIVTGVVDTHVRPEHSAKQHIALNDLCLVLRKSEPYTEGLLKDIQPLAQPCSLKDIVPQNSNEGWEEEAKVEFLKMVNNKAVSMKVFREEDGVLIVDLQKPPPNKISSDMPVSLRDALVFMELAKFKSQSLRSHFEKNTTLHYHPPILPKEMTDVSVTVCHINSPGDFYLQLIEGLDILFLLKTIEEFYKSEDGENLEILCPVQDQACVAKFEDGIWYRAKVIGLPGHQEVEVKYVDFGNTAKITIKDVRKIKDEFLNAPEKAIKCKLAYIEPYKRTMQWSKEAKEKFEEKAQDKFMTCSVIKILEDNVLLVELFDSLGAPEMTTTSINDQLVKEGLASYEIGYILKDNSQKHIEVWDPSPEEIISNEVHNLNPVSAKSLPNENFQSLYNKELPVHICNVISPEKIYVQWLLTENLLNSLEEKMIAAYENSKWEPVKWENDMHCAVKIQDKNQWRRGQIIRMVTDTLVEVLLYDVGVELVVNVDCLRKLEENLKTMGRLSLECSLVDIRPAGGSDKWTATACDCLSLYLTGAVATIILQVDSEENNTTWPLPVKIFCRDEKGERVDVSKYLIKKGLALRERRINNLDNSHSLSEKSLEVPLEQEDSVVTNCIKTNFDPDKKTADIISEQKVSEFQEKILEPRTTRGYKPPAIPNMNVFEATVSCVGDDGTIFVVPKLSEFELIKMTNEIQSNLKCLGLLEPYFWKKGEACAVRGSDTLWYRGKVMEVVGGAVRVQYLDHGFTEKIPQCHLYPILLYPDIPQFCIPCQLHNTTPVGNVWQPDAIEVLQQLLSKRQVDIHIMELPKNPWEKLSIHLYFDGMSLSYFMAYYKYCTSEHTEEMLKEKPRSDHDKKYEEEQWEIRFEELLSAETDTPLLPPYLSSSLPSPGELYAVQVKHVVSPNEVYICLDSIETSNQSNQHSDTDDSGVSGESESESLDEALQRVNKKVEALPPLTDFRTEMPCLAEYDDGLWYRAKIVAIKEFNPLSILVQFVDYGSTAKLTLNRLCQIPSHLMRYPARAIKVLLAGFKPPLRDLGETRIPYCPKWSMEALWAMIDCLQGKQLYAVSMAPAPEQIVTLYDDEQHPVHMPLVEMGLADKDE

SEQ ID NO: 22 - protein, 474 aa, Homo sapiens
MSLQSPSRLLELAGQSLLRNQFLTIFILDELPREVFPLMFMEASSMRHFEALKLMVQAWPFLRLPLGSLMKTPHLETLQAVLKGLDTLLAQKLRPRRWKLQVLDLRDVDGNFWTIWSGARALSCSPEAMSKRQTVEDYPRTGEHQPLKVFIDLCQKESTLDECLSYLCRWIHYRRGLVHLCCNKVQNYSMPTSSFRNLLKRVYPDSIQELEIKRKCSLNKTGKFAPYLSQMSNLRKLFLAFGYDDELYVSGQQQFVPDLDCPFLCLYYPQMLYIRKISNIKEHLEHLLRCLKNPLGTFIFCHAYLADQDMECLSQYPSLSQLKELHLIHILMWTTNLEPLGALLEKVAATLEILTLKDCQIQDSQLRVLLPALSRCSQLTTFYFRGNETSTNALKDLLCHTGGLSKLGLELYPAPLECLDNRGHVNWEILAPIRAELMCTLREVRQPKRIFFGPIPCPSCGSWPSEKVDFHLCS

SEQ ID NO: 23 - protein, 487 aa, Homo sapiens
MAAALPRTLGELQLYRILQKANLLSYFDAFIQQGGDDVQQLCEAGEEEFLEIMALVGMASKPLHVRRLQKALRDWVTNPGLFNQPLTSLPVSSIPIYKLPEGSPTWLGISCSSYERSSNAREPHLKIPKCAATTCVQSLGQGKSDVVGSLALQSVGESRLWQGHHATESEHSLSPADLGSPASPKESSEALDAAAALSVAECVERMAPTLPKSDLNEVKELLKTNKKLAKMIGHIFEMNDDDPHKEEEIRKYSAIYGRFDSKRKDGKHLTLHELTVNEAAAQLCVKDNALLTRRDELFALARQISREVTYKYTYRTTKSKCGERDELSPKRIKVEDGFPDFQDSVQTLFQQARAKSEELAALSSQQPEKVMAKQMEFLCNQAGYERLQHAERRLSAGLYRQSSEEHSPNGLTSDNSDGQGERPLNLRMPNLQNRQPHHFVVDGELSRLYPSEAKSHSSESLGILKDYPHSAFTLEKKVIKTEPEDSR

SEQ ID NO: 24 - protein, 330 aa, Homo sapiens
MEGAPPGSLALRLLLFVALPASGWLTTGAPEPPPLSGAPQDGIRINVTTLKDDGDISKQQVVLNITYESGQVYVNDLPVNSGVTRISCQTLIVKNENLENLEEKEYFGIVSVRILVHEWPMTSGSSLQLIVIQEEVVEIDGKQVQQKDVTEIDILVKNRGVLRHSNYTLPLEESMLYSISRDSDILFTLPNLSKKESVSSLQTTSQYLIRNVETTVDEDVLPGKLPETPLRAEPPSSYKVMCQWMEKFRKDLCRFWSNVFPVFFQFLNIMVVGITGAAVVITILKVFFPVSEYKGILQLDKVDVIPVTAINLYPDGPEKRAENLEDKTCI

SEQ ID NO: 25 - protein, 816 aa, Homo sapiens
MSRPQGLLWLPLLFTPVCVMLNSNVLLWLTALAIKFTLIDSQAQYPVVNTNYGKIRGLRTPLPNEILGPVEQYLGVPYASPPTGERRFQPPEPPSSWTGIRNTTQFAAVCPQHLDERSLLHDMLPIWFTANLDTLMTYVQDQNEDCLYLNIYVPTEDDIHDQNSKKPVMVYIHGGSYMEGTGNMIDGSILASYGNVIVITINYRLGILGFLSTGDQAAKGNYGLLDQIQALRWIEENVGAFGGDPKRVTIFGSGAGASCVSLLTLSHYSEGLFQKAIIQSGTALSSWAVNYQPAKYTRILADKVGCNMLDTTDMVECLRNKNYKELIQQTITPATYHIAFGPVIDGDVIPDDPQILMEQGEFLNYDIMLGVNQGEGLKFVDGIVDNEDGVTPNDFDFSVSNFVDNLYGYPEGKDTLRETIKFMYTDWADKENPETRRKTLVALFTDHQWVAPAVATADLHAQYGSPTYFYAFYHHCQSEMKPSWADSAHGDEVPYVFGIPMIGPTELFSCNFSKNDVMLSAVVMTYWTNFAKTGDPNQPVPQDTKFIHTKPNRFEEVAWSKYNPKDQLYLHIGLKPRVRDHYRATKVAFWLELVPHLHNLNEIFQYVSTTTKVPPPDMTSFPYGTRRSPAKIWPTTKRPAITPANNPKHSKDPHKTGPEDTTVLIETKRDYSTELSVTIAVGASLLFLNILAFAALYYKKDKRRHETHRRPSPQRNTTNDIAHIQNEEIMSLQMKQLEHDHECESLQAHDTLRLTCPPDYTLTLRRSPDDIPLMTPNTITMIPNTLTGMQPLHTFNTFSGGQNSTNLPHGHSTTRV

SEQ ID NO: 26 - protein, 297 aa, Homo sapiens
MSRFKFIDIGINLTDPMFRGIYRGVQKHQDDLQDVIGRAVEIGVKKFMITGGNLQDSKDALHLAQTNGMFFSTVGCHPTRCGEFEKNNPDLYLKELLNLAENNKGKVVAIGECGLDFDRLQFCPKDTQLKYFEKQFELSEQTKLPMFLHCRNSHAEFLDIMKRNRDRCVGGVVHSFDGTKEAAAALIDLDLYIGFNGCSLKTEANLEVLKSIPSEKLMIETDAPWCGVKSTHAGSKYIRTAFPTKKKWESGHCLKDRNEPCHIIQILEIMSAVRDEDPLELANTLYNNTIKVFFPGI

SEQ ID NO: 27 - protein, 214 aa, Homo sapiens
MGAAHSASEEVRELEGKTGFSSDQIEQLHRRFKQLSGDQPTIRKENFNNVPDLELNPIRSKIVRAFFDNRNLRKGPSGLADEINFEDFLTIMSYFRPIDTTMDEEQVELSRKEKLRFLFHMYDSDSDGRITLEEYRNVVEELLSGNPHIEKESARSIADGAMMEAASVCMGQMEPDQVYEGITFEDFLKIWQGIDIETKMHVRFLNMETMALCH

SEQ ID NO: 28 - protein, 97 aa, Homo sapiens
MDKQSSAGGVKRSVPCDSNEANEMMPETSSGYSDPQPAPKKLKTSESSTILVVRYRRNFKRTSPEELVNDHARKNRINPLQMEEEEFMEIMVEIPAK

SEQ ID NO: 29 - protein, 287 aa, Homo sapiens
MELERIVSAALLAFVQTHLPEADLSGLDEVIFSYVLGVLEDLGPSGPSEENFDMEAFTEMMEAYVPGFAHIPRGTIGDMMQKLSGQLSDARNKENLQPQSSGVQGQVPISPEPLQRPEMLKEETRSSAAAAADTQDEATGAEEELLPGVDVLLEVFPTCSVEQAQWVLAKARGDLEEAVQMLVEGKEEGPAAWEGPNQDLPRRLRGPQKDELKSFILQKYMMVDSAEDQKIHRPMAPKEAPKKLIRYIDNQVVSTKGERFKDVRNPEAEEMKATYINLKPARKYRFH

SEQ ID NO: 30 - protein, 324 aa, Homo sapiens
MSSEAETQQPPAAPPAAPALSAADTKPGTTGSGAGSGGPGGLTSAAPAGGDKKVIATKVLGTVKWFNVRNGYGFINRNDTKEDVFVHQTAIKKNNPRKYLRSVGDGETVEFDVVEGEKGAEAANVTGPGGVPVQGSKYAADRNHYRRYPRRRGPPRNYQQNYQNSESGEKNEGSESAPEGQAQQRRPYRRRRFPPYYMRRPYGRRPQYSNPPVQGEVMEGADNQGAGEQGRPVRQNMYRGYRPRFRRGPPRQRQPREDGNEEDKENQGDETQGQQPPQRRYRRNFNYRRRRPENPKPQDGKETKAADPPAENSSAPEAEQGGAE

SEQ ID NO: 31 - protein, 93 aa, Homo sapiens
MLTELEKALNSIIDVYHKYSLIKGNFHAVYRDDLKKLLETECPQYIRKKGADVWFKELDINTDGAVNFQEFLILVIKMGVAAHKKSHEESHKE

SEQ ID NO: 32 - protein, 434 aa, Homo sapiens
MSILKIHAREIFDSRGNPTVEVDLFTSKGLFRAAVPSGASTGIYEALELRDNDKTRYMGKGVSKAVEHINKTIAPALVSKKLNVTEQEKIDKLMIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 33 - protein, 146 aa, Homo sapiens
MAGELTPEEEAQYKKAFSAVDTDGNGTINAQELGAALKATGKNLSEAQLRKLISEVDSDGDGEISFQEFLTAAKKARAGLEDLQVAFRAFDQDGDGHITVDELRRAMAGLGQPLPQEELDAMIREADVDQDGRVNYEEFARMLAQE

SEQ ID NO: 34 - protein, 107 aa, Homo sapiens
MSESSSKSSQPLASKQEKDGTEKRGRGRPRKQPPVSPGTALVGSQKEPSEVPTPKRPRGRPKGSKNKGAAKTRKTTTTPGRKPRGRPKKLEKEEEEGISQESSEEEQ

SEQ ID NO: 35 - protein, 319 aa, Homo sapiens
MGIWQRLLLFGGVSLRAGGGATAPLGGSRAMVCGRQLSGAGSETLKQRRTQIMSRGLPKQKPIEGVKQVIVVASGKGGVGKSTTAVNLALALAANDSSKAIGLLDVDVYGPSVPKMMNLKGNPELSQSNLMRPLLNYGIACMSMGFLVEESEPVVWRGLMVMSAIEKLLRQVDWGQLDYLVVDMPPGTGDVQLSVSQNIPITGAVIVSTPQDIALMDAHKGAEMFRRVHVPVLGLVQNMSVFQCPKCKHKTHIFGADGARKLAQTLGLEVLGDIPLHLNIREASDTGQPIVFSQPESDEAKAYLRIAVEVVRRLPSPSE

SEQ ID NO: 36 - protein, 601 aa, Homo sapiens
MAEPSGAETRPPIRVTVKTPKDKEEIVICDRASVKEFKEEISRRFKAQQDQLVLIFAGKILKDGDTLNQHGIKDGLTVHLVIKTPQKAQDPAAATASSPSTPDPASAPSTTPASPATPAQPSTSGSASSDAGSGSRRSSGGGPSPGAGEGSPSATASILSGFGGILGLGSLGLGSANFMELQQQMQRQLMSNPEMLSQIMENPLVQDMMSNPDLMRHMIMANPQMQQLMERNPEISHMLNNPELMRQTMELARNPAMMQEMMRNQDRALSNLESIPGGYNALRRMYTDIQEPMFSAAREQFGNNPFSSLAGNSDSSSSQPLRTENREPLPNPWSPSPPTSQAPGSGGEGTGGSGTSQVHPTVSNPFGINAASLGSGMFNSPEMQALLQQISENPQLMQNVISAPYMRSMMQTLAQNPDFAAQMMVNVPLFAGNPQLQEQLRLQLPVFLQQMQNPESLSILTNPRAMQALLQIQQGLQTLQTEAPGLVPSLGSFGISRTPAPSAGSNAGSTPEAPTSSPATPATSSPTGASSAQQQLMQQMIQLLAGSGNSQVQTPEVRFQQQLEQLNSMGFINREANLQALIATGGDINAAIERLLGSQLS

SEQ ID NO: 37 - protein, 218 aa, Homo sapiens
MGTRDDEYDYLFKVVLIGDSGVGKSNLLSRFTRNEFNLESKSTIGVEFATRSIQVDGKTIKAQIWDTAGQERYRAITSAYYRGAVGALLVYDIAKHLTYENVERWLKELRDHADSNIVIMLVGNKSDLRHLRAVPTDEARAFAEKNNLSFIETSALDSTNVEEAFKNILTEIYRIVSQKQIADRAAHDESPGNNVVDISVPPTTDGQKPNKLQCCQNL

SEQ ID NO: 38 - protein, 215 aa, Homo sapiens
MVLLTMIARVADGLPLAASMQEDEQSGRDLQQYQSQAKQLFRKLNEQSPTRCTLEAGAMTFHYIIEQGVCDLVLCEAAFPKTLAFAYLEDLHSEFDEQHGKKVPTVSRPYSFIEFDTFIQKTKKLYIDSCARRNLGSINTELQDVQRIMVANIEEVLQRGEALSALDSKANNLSSLSKKYRQDAKYLNMHSTYAKLAAVAVFFIMLIVYVRFWWL

SEQ ID NO: 39 - protein, 360 aa, Homo sapiens
MPKFKQRRRKLKAKAERLFKKKEASHFQSKLITPPPPPPSPERVGISSIDISQSRSWLTSSWNFNFPNIRDAIKLWTNRVWSIYSWCQNCITQSLEVLKDTIFPSRICHRELYSVKQQFCILESKLCKLQEALKTISESSSCPSCGQTCHMSGKLTNVPACVLITPGDSKAVLPPTLPQPASHFPPPPPPPPLPPPPPPLAPVLLRKPSLAKALQAGPLKKDGPMQITVKDLLTVKLKKTQSLDEKRKLIPSPKARNPLVTVSDLQHVTLKPNSKVLSTRVTNVLITPGKSQMDLRKLLRKVDVERSPGGTPLTNKENMETGTGLTPVMTQALRRKFQLAHPRSPTPTLPLSTSSFDEQN

SEQ ID NO: 40 - protein, 235 aa, Homo sapiens
MRTQVYEGLCKNYFSLAVLQRDRIKLLFFDILVFLSVFLLFLLFLVDIMANNTTSLGSPWPENFWEDLIMSFTVSMAIGLVLGGFIWAVFICLSRRRRASAPISQWSSSRRSRSSYTHGLNRTGFYRHSGCERRSNLSLASLTFQRQASLEQANSFPRKSSFRASTFHPFLQCPPLPVETESQLVTLPSSNISPTISTSHSLSRPDYWSSNSLRVGLSTPPPPAYESIIKAFPDS

SEQ ID NO: 41 - protein, 172 aa, Homo sapiens
MASNFKKANMASSSQRKRMSPKPELTEEQKQEIREAFDLFDADGTGTIDVKELKVAMRALGFEPKKEEIKKMISEIDKEGTGKMNFGDFLTVMTQKMSEKDTKEEILKAFKLFDDDETGKISFKNLKRVAKELGENLTDEELQEMIDEADRDGDGEVSEQEFLRIMKKTSLY

SEQ ID NO: 42 - protein, 525 aa, Homo sapiens
MHRAPSPTAEQPPGGGDSARRTLQPRLKPSARAMALPRTLGELQLYRVLQRANLLSYYETFIQQGGDDVQQLCEAGEEEFLEIMALVGMATKPLHVRRLQKALREWATNPGLFSQPVPAVPVSSIPLFKISETAGTRKGSMSNGHGSPGEKAGSARSFSPKSPLELGEKLSPLPGGPGAGDPRIWPGRSTPESDVGAGGEEEAGSPPFSPPAGGGVPEGTGAGGLAAGGTGGGPDRLEPEMVRMVVESVERIFRSFPRGDAGEVTSLLKLNKKLARSVGHIFEMDDNDSQKEEEIRKYSIIYGRFDSKRREGKQLSLHELTINEAAAQFCMRDNTLLLRRVELFSLSRQVARESTYLSSLKGSRLHPEELGGPPLKKLKQEVGEQSHPEIQQPPPGPESYVPPYRPSLEEDSASLSGESLDGHLQAVGSCPRLTPPPADLPLALPAHGLWSRHILQQTLMDEGLRLARLVSHDRVGRLSPCVPAKPPLAEFEEGLLDRCPAPGPHPALVEGRRSSVKVEAEASRQ

SEQ ID NO: 43 - protein, 852 aa, Homo sapiens
MLSGKKAAAAAAAAAAAATGTEAGPGTAGGSENGSEVAAQPAGLSGPAEVGPGAVGERTPRKKEPPRASPPGGLAEPPGSAGPQAGPTVVPGSATPMETGIAETPEGRRTSRRKRAKVEYREMDESLANLSEDEYYSEEERNAKAEKEKKLPPPPPQAPPEEENESEPEEPSGVEGAAFQSRLPHDRMTSQEAACFPDIISGPQQTQKVFLFIRNRTLQLWLDNPKIQLTFEATLQQLEAPYNSDTVLVHRVHSYLERHGLINFGIYKRIKPLPTKKTGKVIIIGSGVSGLAAARQLQSFGMDVTLLEARDRVGGRVATFRKGNYVADLGAMVVTGLGGNPMAVVSKQVNMELAKIKQKCPLYEANGQAVPKEKDEMVEQEFNRLLEATSYLSHQLDFNVLNNKPVSLGQALEVVIQLQEKHVKDEQIEHWKKIVKTQEELKELLNKMVNLKEKIKELHQQYKEASEVKPPRDITAEFLVKSKHRDLTALCKEYDELAETQGKLEEKLQELEANPPSDVYLSSRDRQILDWHFANLEFANATPLSTLSLKHWDQDDDFEFTGSHLTVRNGYSCVPVALAEGLDIKLNTAVRQVRYTASGCEVIAVNTRSTSQTFIYKCDAVLCTLPLGVLKQQPPAVQFVPPLPEWKTSAVQRMGFGNLNKVVLCFDRVFWDPSVNLFGHVGSTTASRGELFLFWNLYKAPILLALVAGEAAGIMENISDDVIVGRCLAILKGIFGSSAVPQPKETVVSRWRADPWARGSYSYVAAGSSGNDYDLMAQPITPGPSIPGAPQPIPRLFFAGEHTIRNYPATVHGALLSGLREAGRIADQFLGAMYTLPRQATPGVPAQQSPSM

SEQ ID NO: 44 - protein, 1428 aa, Homo sapiens
MENEPDHENVEQSLCAKTSEEELNKSFNLEASLSKFSYIDMDKELEFKNDLIDDKEFDIPQVDTPPTLESILNETDDEDESFILEDPTLLNIDTIDSHSYDTSSVASSDSGDRTNLKRKKKLPDSFSLHGSVMRHSLLKGISAQIVSAADKVDAGLPTAIAVSSLIAVGTSHGLALIFGKDQNQALRLCLGSTSVGGQYGAISALSINNDCSRLLCGFAKGQITMWDLASGKLLRSITDAHPPGTAILHIKFTDDPTLAICNDSGGSVFELTFKRVMGVRTCESRCLFSGSKGEVCCIEPLHSKPELKDHPITQFSLLAMASLTKILVIGLKPSLKVWMTFPYGRMDPSSVPLLAWHFVAVQNYVNPMLAFCRGDVVHFLLVKRDESGAIHVTKQKHLHLYYDLINFTWINSRTVVLLDSVEKLHVIDRQTQEELETVEISEVQLVYNSSHFKSLATGGNVSQALALVGEKACYQSISSYGGQIFYLGTKSVYVMMLRSWRERVDHLLKQDCLTEALALAWSFHEGKAKAVVGLSGDASKRKAIVADRMVEILFHYADRALKKCPDQGKIQVMEQHFQDMVPVIVDYCLLLQRKDLLFSQMYDKLSENSVAKGVFLECLEPYILSDKLVGITPQVMKDLIVHFQDKKLMENVEALIVHMDITSLDIQQVVLMCWENRLYDAMIYVYNRGMNEFISPMEKLFRVIAPPLNAGKTLTDEQVVMGNKLLVYISCCLAGRAYPLGDIPEDLVPLVKNQVFEFLIRLHSAEASPEEEIYPYIRTLLHFDTREFLNVLALTFEDFKNDKQAVEYQQRIVDILLKVMVENSDFTPSQVGCLFTFLARQLAKPDNTLFVNRTLFDQVLEFLCSPDDDSRHSERQQVLLELLQAGGIVQFEESRLIRMAEKAEFYQICEFMYEREHQYDKIIDCYLRDPLREEEVFNYIHNILSIPGHSAEEKQSVWQKAMDHIEELVSLKPCKAAELVATHFSGHIETVIKKLQNQVLLFKFLRSLLDPREGIHVNQELLQISPCITEQFIELLCQFNPTQVIETLQVLECYRLEETIQITQKYQLHEVTAYLLEKKGDIHGAFLIMLERLQSKLQEVTHQGENTKEDPSLKDVEDTMVETIALCQRNSHNLNQQQREALWFPLLEAMMAPQKLSSSAIPHLHSEALKSLTMQVLNSMAAFIALPSILQRILQDPVYGKGKLGEIQGLILGMLDTFNYEQTLLETTTSLLNQDLHWSLCNLRASVTRGLNPKQDYCSICLQQYKRRQEMADEIIVFSCGHLYHSFCLQNKECTVEFEGQTRWTCYKCSSSNKVGKLSENSSEIKKGRITPSQVKMSPSYHQSKGDPTAKKGTSEPVLDPQQIQAFDQLCRLYRGSSRLALLTELSQNRSSESYRPFSGSQSAPAFNSIFQNENFQLQLIPPPVTED

SEQ ID NO: 45 - protein, 4243 aa, Homo sapiens
MGHLWLLGIWGLCGLLLCAADPSTDGSQIIPKVTEIIPKYGSINGATRLTIRGEGFSQANQFNYGVDNAELGNSVQLISSFQSITCDVEKDASHSTQITCYTRAMPEDSYTVRVSVDGVPVTENNTCKGHINSWECTFNAKSFRTPTIRSITPLSGTPGTLITIQGRIFTDVYGSNIALSSNGKNVRILRVYIGGMPCELLIPQSDNLYGLKLDHPNGDMGSMVCKTTGTFIGHHNVSFILDNDYGRSFPQKMAYFVSSLNKIAMFQTYAEVTMIFPSQGSIRGGTTLTISGRFFDQTDFPVRVLVGGEPCDILNVTENSICCKTPPKPHILKTVYPGGRGLKLEVWNNSRPIRLEEILEYNEKTPGYMGASWVDSASYIWLMEQDTFVARFSGFLVAPDSDVYRFYIKGDDRYAIYFSQTGLPEDKVRIAYHSANANSYFSSPTQRSDDIHLQKGKEYYIEILLQEYRLSAFVDVGLYQYRNVYTEQQTGDAVNEEQVIKSQSTILQEVQVITLENWETTNAINEVQKIKVTSPCVEANSCSLYQYRLIYNMEKTVFLPADASEFILQSALNDLWSIKPDTVQVIRTQNPQSYVYMVTFISTRGDFDLLGYEVVEGNNVTLDITEQTKGKPNLETFTLNWDGIASKPLTLWSSEAEFQGAVEEMVSTKCPPQIANFEEGFVVKYFRDYETDFNLEHINRGQKTAETDAYCGRYSLKNPAVLFDSADVKPNRRPYGDILLFPYNQLCLAYKGFLANYIGLKFQYQDNSKITRSTDTQFTYNFAYGNNWTYTCIDLLDLVRTKYTGTNVSLQRISLHKASESQSFYVDVVYIGHTSTISTLDEMPKRRLPALANKGIFLEHFQVNQTKTNGPTMTNQYSVTMTSYNCSYNIPMMAVSFGQIITHETENEFVYRGNNWPGESKIHIQRIQAASPPLSGSFDIQAYGHILKGLPAAVSAADLQFALQSLEGMGRISVTREGTCAGYAWNIKWRSTCGKQNLLQINDSNIIGEKANMTVTRIKEGGLFRQHVLGDLLRTPSQQPQVEVYVNGIPAKCSGDCGFTWDSNITPLVLAISPSQGSYEEGTILTIVGSGFSPSSAVTVSVGPVGCSLLSVDEKELKCQILNGSAGHAPVAVSMADVGLAQNVGGEEFYFVYQSQISHIWPDSGSIAGGTLLTLSGFGFNENSKVLVGNETCNVIEGDLNRITCRTPKKTEGTVDISVTTNGFQATARDAFSYNCLQTPIITDFSPKVRTILGEVNLTIKGYNFGNELTQNMAVYVGGKTCQILHWNFTDIRCLLPKLSPGKHDIYVEVRNWGFASTRDKLNSSIQYVLEVTSMFPQRGSLFGGTEITIRGFGFSTIPAENTVLLGSIPCNVTSSSENVIKCILHSTGNIFRITNNGKDSVHGLGYAWSPPVLNVSVGDTVAWHWQTHPFLRGIGYRIFSVSSPGSVIYDGKGFTSGRQKSTSGSFSYQFTSPGIHYYSSGYVDEAHSIFLQGVINVLPAETRHIPLHLFVGRSEATYAYGGPENLHLGSSVAGCLATEPLCSLNNTRVKNSKRLLFEVSSCFSPSISNITPSTGTVNELITIIGHGFSNLPWANKVTIGSYPCVVEESSEDSITCHIDPQNSMDVGIRETVTLTVYNLGTAINTLSNEFDRRFVLLPNIDLVLPNAGSTTGMTSVTIKGSGFAVSSAGVKVLMGHFPCKVLSVNYTAIECETSPAAQQLVDVDLLIHGVPAQCQGNCTFSYLESITPYITGVFPNSVIGSVKVLIEGEGLGTVLEDIAVFIGNQQFRAIEVNENNITALVTPLPVGHHSVSVVVGSKGLALGNLTVSSPPVASLSPTSGSIGGGTTLVITGNGFYPGNTTVTIGDEPCQIISINPNEVYCRTPAGTTGMVDVKIFVNTIAYPPLLFTYALEDTPFLRGIIPSRGPPGTEIEITGSNFGFEILEISVMINNIQCNVTMANDSVVQCIVGDHAGGTFPVMMHHKTKGSAMSTVVFEYPLNIQNINPSQGSFGGGQTMTVTGTGFNPQNSIILVCGSECAIDRLRSDYTTLLCEIPSNNGTGAEQACEVSVVNGKDLSQSMTPFTYAVSLTPLITAVSPKRGSTAGGTRLTVVGSGFSENMEDVHITIAEAKCDVEYSNKTHIICMTDAHTLSGWAPVCVHIRGVGMAKLDNADFLYVDAWSSNFSWGGKSPPEEGSLVVITKGQTILLDQSTPILKMLLIQGGTLIFDEADIELQAENILITDGGVLQIGTETSPFQHKAVITLHGHLRSPELPVYGAKTLAVREGILDLHGVPVPVTWTRLAHTAKAGERILILQEAVTWKPGDNIVIASTGHRHSQGENEKMTIASVSADGINITLSNPLNYTHLGITVTLPDGTLFEARAEVGILTRNILIRGSDNVEWNNKIPACPDGFDTGEFATQTCLQGKFGEEIGSDQFGGCVMFHAPVPGANMVTGRIEYVEVFHAGQAFRLGRYPIHWHLLGDLQFKSYVRGCAIHQAYNRAVTIHNTHHLLVERNIIYDIKGGAFFIEDGIEHGNILQYNLAVFVQQSTSLLNDDVTPAAFWVTNPNNTIRHNAVAGGTHFGFWYRMNNHPDGPSYDRNICQKRVPLGEFFNNTVHSQGWFGMWIFEEYFPMQTGSCTSTVPAPAIFNSLTTWNCQKGAEWVNGGALQFHNFVMVNNYEAGIETKRILAPYVGGWGETNGAVIKNAKIVGHLDELGMGSAFCTAKGLVLPFSEGLTVSSVHFMNFDRPNCVALGVTSISGVCNDRCGGWSAKFVDVQYSHTPNKAGFRWEHEMVMIDVDGSLTGHKGHTVIPHSSLLDPSHCTQEAEWSIGFPGSVCDASVSFHRLAFNQPSPVSLLEKDVVLSDSFGTSIIPFQKKRLTHMSGWMALIPNANHINWYFKGVDHITNISYTSTFYGFKEEDYVIISHNFTQNPDMFNIIDMRNGSSNPLNWNTSKNGDWHLEANTSTLYYLVSGRNDLHQSQLISGNLDPDVKDVVINFQAYCCILQDCFPVHPPSRKPIPKKRPATYNLWSNDSFWQSSRENNYTVPHPGANVIIPEGTWIVADIDMPSMERLIIWGVLELEDKYNVGAAESSYREVVLNATYISLQGGRLIGGWEDNPFKGDLKIVLRGNHTTQDWALPEGPNQGAKVLGVFGELDLHGIPHSIYKTKLSETAFAGSKVLSLMDAVDWQEGEEIVITTTSYDFHQTETRSIVKILHDHKILILNDSLSYTHFAEKYHVPGTGESYTLAADVGILSRNIKIVGEDYPGWSEDSFGARVLVGSFTENMMTFKGNARISNVEFYHSGQEGFRDSTDPRYAVTFLNLGQIQEHGSSYIRGCAFHHGFSPAIGVFGTDGLDIDDNIIHFTVGEGIRIWGNANRVRGNLIALSVWPGTYQNRKDLSSTLWHAAIEINRGTNTVLQNNVVAGFGRAGYRIDGEPCPGQFNPVEKWFDNEAHGGLYGIYMNQDGLPGCSLIQGFTIWTCWDYGIYFQTTESVHIYNVTLVDNGMAIFPMIYMPAAISHKISSKNVQIKSSLIVGSSPGFNCSDVLTNDDPNIELTAAHRSPRSPSGGRSGICWPTFASAHNMAPRKPHAGIMSYNAISGLLDISGSTFVGFKNVCSGETNVIFITNPLNEDLQHPIHVKNIKLVDTTEQSKIFIHRPDISKVNPSDCVDMVCDAKRKSFLRDIDGSFLGNAGSVIPQAEYEWDGNSQVGIGDYRIPKAMLTFLNGSRIPVTEKAPHKGIIRDSTCKYLPEWQSYQCFGMEYAMMVIESLDPDTETRRLSPVAIMGNGYVDLINGPQDHGWCAGYTCQRRLSLFHSIVALNKSYEVYFTGTSPQNLRLMLLNVDHNKAVLVGIFFSTLQRLDVYVNNLLVCPKTTIWNAQQKHCELNNHLYKDQFLPNLDSTVLGENYFDGTYQMLYLLVKGTIPVEIHTATVIFVSFQLSVATEDDFYTSHNLVKNLALFLKIPSDKIRISKIRGKSLRRKRSMGFIIEIEIGDPPIQFISNGTTGQMQLSELQEIAGSLGQAVILGNISSILGFNISSMSITNPLPSPSDSGWIKVTAQPVERSAFPVHHVAFVSSLLVITQPVAAQPGQPFPQQPSVKATDSDGNCVSVGITALTLRAILKDSNNNQVNGLSGNTTIPFSSCWANYTDLTPLRTGKNYKIEFILDNVVGVESRTFSLLAESVSSSGSSSSSNSKASTVGTYAQIMTVVISCLVGRMWLLEIFMAAVSTLNITLRSY

SEQ ID NO: 46 - protein, 12 aa, synthetic construct
  REGION 1..12: note = 12-mer peptide selected from a combinatorial phage display library, wherein the peptide is expressed on the phage capsid and binds the BF944 monoclonal antibody, also referred to as mimotope
WHFEFLNIMVNN

SEQ ID NO: 47 - protein, 12 aa, synthetic construct
  REGION 1..12: note = 12-mer peptide selected from a combinatorial phage display library, wherein the peptide is expressed on the phage capsid and binds the BF944 monoclonal antibody, also referred to as mimotope
SVGDDVAAGVYG

SEQ ID NO: 48 - protein, 12 aa, synthetic construct
  REGION 1..12: note = 12-mer peptide selected from a combinatorial phage display library, wherein the peptide is expressed on the phage capsid and binds the BF944 monoclonal antibody, also referred to as mimotope
RVFETPSMFKER

(19) United States
(12) Patent Application Publication
(10) Pub. No.: US 2025/0334576 A1
(43) Pub. Date: Oct. 30, 2025

(54) MARKER FOR CANCER DETECTION (BC / or BF9)

(71) Applicant: MILAGEN, INC., EMERYVILLE, CA (US)

(72) Inventor: Moncef Jendoubi, San Francisco, CA (US)

(21) Appl. No.: 18/957,475

(22) Filed: Nov. 22, 2024

Related U.S. Application Data
(60) Provisional application No. 63/602,300, filed on Nov. 22, 2023.

(51) Int. Cl. G01N 33/574; G01N 33/68

(52) U.S. Cl. CPC G01N 33/57415; G01N 33/57488; G01N 33/6893

(57) ABSTRACT

Biomarkers can be assessed for a variety of uses, including screening, detection, diagnosis, prognosis, risk prediction, disease progression, recurrence, selection of treatment, therapy response, to evaluate a subject's health status, whether the subject presents with no evidence of disease, or a benign or malignant condition such as cancer. Compositions (antibodies, polypeptide and polynucleotide markers) and methods are provided herein, which find application in the early detection of cancer, in the early detection of disease relapse and in monitoring therapy response.

### REFERENCE TO SEQUENCE LISTING

In accordance with 37 CFR 1.831 (2) a sequence listing is incorporated herein by reference. The sequence listing is entitled 2504-0003U, was created on Nov. 21, 2024, and is 47 KB.

### BACKGROUND

Over a million and a half estimated new cancer cases (1,638,910) in the US in 2012 caused over half a million (577,190) deaths. Over a lifetime, roughly half of all people between the ages of 50 to 70 will get some form of cancer. Cancer is the second leading cause of death after heart disease. The overall cost of cancer treatment exceeds half a trillion dollars and is constantly increasing.

The four major cancers in the US are breast, prostate, lung and colorectal (Siegel R et al., Cancer statistics, 2012, CA Cancer J Clin 62:10-29, 2012). One of the most important factors affecting the survival rate of all cancers is early detection. For many cancers, detection at the earliest stages yields survival rates greater than 90%, while detection at the later stages often causes survival rates to fall below 10%. In most

cases, cancer is not detected until a proliferation of cancer cells is physically quite large, such as when an excess growth of tissue creates a lump or other mass that can be seen or felt by a cancer patient or when this mass causes pain or altered function in surrounding tissues or organs.

However, the earliest stages of cancer cause profound changes in the basic physiology of a patient, including changes at the genetic level. While excess cell growth itself causes fundamental changes, other physiological mechanisms are also affected when the cancer grows and spreads throughout the body. Changes in a cancer patients' DNA such as chromosomal alterations, alterations in gene sequences, and altered gene expression patterns also lead to modifications in protein expression. These changes in protein expression at the cellular level correlate with subtle changes in organs, tissues, and body fluids.

Although it is well recognized that a large number of proteins that are involved in the onset and development of cancer are fundamentally altered in terms of their structure, function, or expression, scientists have had limited success in identifying specific proteins that are uniquely associated with the development of cancer and are not found in normal patients. If such proteins could be reliably identified, detection of the proteins would be a valuable tool for the early detection of cancer leading to increased cancer survival rates in the entire population.

Where a particular protein is expressed only in cancer patients, or is expressed in a unique chemical form, or has any other distinguishing feature that distinguishes normal from cancer patients, such a compound may be called a “cancer marker” or “biomarker.” For many years, doctors and scientists have searched for cancer markers that uniquely identify the earliest onset of cancer. Ideally, these markers would not be present in other diseases or in benign conditions such that detection of such a marker would provide a reliable indicator that patient was in the earliest stages of developing cancer. In addition to early detection, these markers could be used to determine a prognosis in a patient, to predict the risk of cancer or relapse, to monitor disease progression or recurrence, to predict a patient's response to surgery or chemotherapy, to assess the effectiveness of treatment, or support patient and clinician's decision making in determining the appropriate course of prevention, surveillance or treatment.

While several potential markers have been analyzed for early cancer detection, very few have actually reached the clinical setting. Recommendations for a number of cancer markers have recently been reviewed by the National Academy of Clinical Biochemistry (NACB) and the American Society of Clinical Oncology (ASCO) panels: in breast cancer (Duffy, 2009; Harris, 2007), colon cancer (Brunner, 2009), lung cancer (Stieber, 2006), prostate cancer (Lilja, 2009), pancreatic cancer (Goggins 2005; Locker, 2006; Duffy, 2010), ovarian cancer (Chan, 2009), and cervical cancer (Gaarenstroom, 2007). A great need remains for early detection cancer markers because many existing markers, such as CEA, CA-15, CA-19, and CA-125, are elevated only in advanced cancer stages. In colon cancer, no effective early stage biomarkers exist, whether tissue or serum-based. While there are methods available for early detection and screening for colon cancer, such as FOBT and colonoscopy, FOBT has limited sensitivity and the latter is an invasive procedure, resulting in only 44% of US adults over the age of 50 undergoing screening (ACS, 2012). No lung cancer or ovarian cancer early detection screening technique is currently available (Stieber, 2006; Smith, 2008). Like many cancers, ovarian cancer is a rather symptomless disease at the early stages, and is mostly detected at advanced stage with imaging and serum CA-125 marker measurements (Chan, 2009), at which point aggressive treatments such as surgery or chemotherapy are less likely to be successful.

PSA screening for prostate cancer in men age 45-50 has been the early detection gold standard for the past few decades (Smith, 2008; Lilja, 2009). However, it is now recommended that patients be informed of the pros and cons of PSA testing prior to screening (ACS, 2012). Where a candidate marker does not adequately distinguish cancer patients from normal patients, for example incorrectly indicating the risk of cancer in patients that are entirely normal, or where the marker fails to detect cancer in a patient, the costs of a misdiagnosis can vastly outweigh the benefits. The limitations of PSA as an early detection marker emphasizes the need for new and better stand-alone biomarkers, or additional biomarkers to supplement and improve current ones.

Some tests have shown an ability to predict whether a tumor in a patient is particularly aggressive. However, these tests typically require a tissue sample taken by an invasive procedure, such as a biopsy from the tumor, for gene expression analysis. These tests are not capable, or practical, for use in early detection in patients having no current symptoms.

Moreover, where the performance of the marker in separating cancer from normal is not adequate, the marker would have no utility when applied to the general population. In other words, while a marker may be used in patients already diagnosed with cancer, or in those at high risk, the ideal marker would be able to reliably distinguish a normal patient from an early cancer patient with enough accuracy that the marker could be used to screen the generally healthy population for early detection of cancer.

Furthermore, while scientists who analyze cancer tissue can readily detect fundamental differences between tumor tissue and regular tissue, those differences are not always attributable to the cancer itself and may be the result of inflammation or other events or conditions that are not directly related to the early onset of cancer. Furthermore, the examination of cancer tissue is not a viable approach for the early detection of cancer in the general population. It is simply impractical, and would be overly burdensome and costly, to surgically remove tissue samples from the general population, even in those patients where a high risk of a tumor exists. Furthermore, the methods to detect cancer often involve expensive and potentially damaging analytical methods, such as x-rays and CT scans that cannot be routinely applied to the population at large and are reserved for only those cases where a clinical diagnosis is already made.

Therefore, an ideal cancer marker would satisfy several different criteria: 1) the marker would identify the onset of cancer at an early stage where the prognosis for a cure and long-term survival are the greatest, 2) the marker would distinguish between normal patients, or those with a benign condition, and early stage cancer patients with very high reliability and would yield limited false negative results, i.e. failing to detect the early development of cancer in patients who in fact have an early stage cancer, and would yield limited false positives, i.e. incorrectly identifying a patient with cancer who is actually cancer free.

Still further, an ideal marker for the early detection of cancer would be simple and inexpensive to detect and could be detected in a patient's body fluid such as blood or urine, such that the test could be performed without a biopsy to remove tissue or other invasive or expensive procedures. Also, an ideal marker could be measured as a simple laboratory test that is conveniently and routinely performed as part of a regular visit to the doctor.

Because a wide variety of blood tests and urinalysis are routinely performed in doctors' offices and medical laboratories, a test kit or method for the early detection of cancer would be a powerful addition to the existing battery of tests performed on patients as part of ordinary health management. Moreover, in patients who are at high risk of developing cancer, i.e. certain patients in the aging population or with a family history or other history indicating a high risk of cancer, the ability to detect and treat cancer at the earliest stages would save millions of lives and preserve billions of dollars in resources otherwise dedicated to treating late stage cancer.

Therefore, an urgent need exists for cancer markers for all types of cancer where the marker enables non-invasive early cancer detection methods, and where tests identifying the marker are accurate, reliable, sensitive and specific, and that can be applied to the asymptomatic general population. If such markers were identified, they could also be used to obtain a prognosis upon detection in the body, to track the progression or metastasis of cancer, to track the treatment response once surgical or drug therapy begins, to identify patients who are free of cancer and thus require regular annual screening, and those in need of more active surveillance.

In the specific case of breast cancer, 246,660 new cases of the invasive type, and 61,000 new cases of the in situ type of breast cancer were diagnosed in a recent year. Breast cancer is the most diagnosed malignancy in women, representing 29% of all new female estimated cancer cases. With 40,450 deaths in a recent year, breast cancer remains the second cause of cancer mortality in women after lung cancer, representing 14% of all female cancer deaths, versus 26% for lung cancer (Siegel, 2016; ACS, 2016).

Mammography is a low dosage x-ray screening procedure that is currently the standard of care for breast cancer detection and is a valuable non-invasive screening method where available. While mammography is currently the best screening modality for early breast cancer detection, decades of use have also revealed its limitations. Mammography sensitivity varies with age and breast density, with a significantly high false-negative rate in the younger patients and in patients having more dense breast tissue. Mammography also does not always discriminate breast cancer from many common benign conditions, leading to a tentative false positive diagnosis that leads to fear, anxiety, and unnecessary additional procedures and expense.

A three-decade analysis of the impact of screening mammography on breast cancer incidence has revealed a reduction of only 8% in late stage breast cancer detection, while contributing to a 31% increase in breast cancer overdiagnosis (Bleyer, 2012).

These limitations have sparked a debate on the risk-benefit analysis underlying the white-scale use of mammography (Heyes, 2009; Smith, 2014). This in turn has prompted segments of the medical community to update their guidelines for the starting age, use and frequency of the mammnography procedure depending on patient age and risk factors (Smith, 2016).

The US Preventive Services Task Force (USPSTF) recommends routine screening by mammography every two years for women 50-74 of age at average risk of breast cancer. Before 50 and after 74, a patient is recommended to discuss the risk/benefit of mammography screening with her health care provider (USPTF, 2016). The American Cancer Society (ACS, 2015; Smith, 2016) recommends annual mammography starting at age 45 for average risk women; switching to mammography every two years after age 55 is optional. The National Comprehensive Cancer Network (NCCN) and The College of Obstetricians and Gynecologists (ACOG) continue to recommend annual mammography starting at age 40 for average risk women.

At present, there are no serum based biomarkers in clinical use for early breast cancer detection and screening. Markers that have been analyzed for potential use in breast cancer screening and diagnosis include serum markers CA15-3 and CA27-29, two overlapping soluble epitopes of MUC1 (Graves, 1998), a heavily glycosylated transmembrane protein present on mucosal epithelia of airway passages, breast and uterus, involved in cell adhesion, signaling and communication. Both markers are elevated in advanced stage of breast and other cancers (i.e. ovarian, pancreatic), and in benign breast and ovary diseases. CEA, a protein restricted to fetal development (Hammerstrom, 1999), is elevated in colorectal and other cancers, such as breast and the gastro-intestinal tract.

Although CA15-3, CA27-29, and CEA have been in wide clinical use in monitoring breast cancer progression as well as prognosis and therapy follow-up (Duffy, 2006, 2009), the American Society of Clinical Oncology (ASCO) guidelines do not recommend the use of these serum markers for breast cancer screening, diagnosis and staging, or for therapy monitoring of stage I-III primary breast cancer. ASCO only recommends CA15-3, CA27-29, and CEA for monitoring therapy efficacy and disease regression in stage IV metastatic breast cancer in conjunction with diagnostic imaging, medical history and physical examination, not as stand-alone markers (Harris, 2007; Khatcheressian, 2013; Runowicz, 2016).

Clinically, breast cancer is a heterogenous disease classified into three major subtypes: luminal A/B, comprising estrogen (ER+) and/or progesterone receptor (PR+) positive tumors, HER2 (HER2+ positive tumors) and triple negative (TNBC; tumors negative for each of these markers). These subtypes differ in patient outcomes and treatments (Perou, 2011; TCGAN, 2012; Prat, 2015).

HER-2, ER and PR have become established markers that are routinely measured in the patient primary tumor or biopsy to provide information on patient prognosis and therapy selection. They have been extensively clinically validated, gaining ASCO recommendation in guiding therapy for women with early-stage or metastatic breast cancer (Harris, 2016; Van Poznak, 2015). The presence of ER/PR (luminal A and B subtypes) and HER2 (HER2 subtype), or their absence (TNBC subtype) in a breast tumor defines breast cancer subtypes, and determines whether the patient will be treated with hormone therapy, chemotherapy or antibody therapy. For instance, HER-2, a transmembrane glycoprotein receptor with tyrosine-kinase activity, is overexpressed on the surface of breast cancer cells as a result of a gene amplification occurring in 25% of the breast cancer patient population. Presence of this marker, assessed by immunohistochemistry (IHC), fluorescence in situ hybridization (FISH) or an assay that detects an extracellular domain released in serum, determines patient susceptibility to Herceptin monoclonal antibody therapy (trastuzumab; Carney, 2007; Lam, 2012). On the other hand ER/PR (estrogen/progesterone receptor) positive breast cancers are candidates for hormone therapy.

The uPA/PAI-1 pair (urokinase-type plasminogen activator and its PA inhibitor type-1) has demonstrated utility in recurrence risk, prognosis and therapy prediction (Duffy, 2014). Like ER/PR, this marker is assessed on tissue biopsies or on the tumor removed at surgery.

Additionally, BRAC1 and BRCA2 are genetic risk assessment markers, occurring in 5-10% of all breast cancer patients. Women carrying BRAC1 and BRCA2 germline mutations have an extremely high lifetime risk of developing breast cancer and ovarian cancer (Couch, 2014).

There are at least six multigene signature tests that have been developed to predict risk of breast cancer recurrence after a primary diagnosis (Weigel, 2010; Harbeck, 2014). Based on DNA microarray analysis of patient tumor samples, they measure the activity level of different gene signatures in preserved patient tissues from biopsy or resection, yielding an algorithm-based risk score of breast cancer recurrence or distant metastasis. They are Oncotype Dx (Genomic Health; 21 genes; Paik, 2004, 2006), Mammaprint (Agendia; 70 genes; Van't Veer, 2002; Van de Vijer, 2002), PAM50 (Nanostring: 58 genes; Wallden, 2015), Mammostrat (5 BMs; Bartlett, 2010), EndoPredict (Sividon Diagnostics/Myriad; 8 genes; Filipits, 2011), and the Breast Cancer Index (bioTheranostics; Sgroi, 2016). These tests also predict the likelihood of benefiting from a given therapy in specific patient subgroups. ASCO recognizes sufficient evidence for clinical utility of the uPA-PAI-1, OncotypeDx, PAM50, EndoPredict and Breast Cancer Index, but not to guide therapy choices (Harris, 2016).

Based on the state of breast cancer biomarkers summarized above: i) serum markers CA15-3, CA27-29, CEA have drawbacks for widespread use in early breast cancer detection, screening, or diagnosis, and are best used for monitoring metastatic breast cancer, and ii) gene signature assays (e.g. OncotypeDx) provide a calculated score that predicts the risk of recurrence and the likelihood of benefiting from a given therapy based on the patient subgroup, but are one-time prognostic tools, not diagnostic or screening assays.

There has been a significant effort to develop novel biomarkers for breast cancer detection. Many approaches have been applied, including DNA/RNA microarrays, 2D gel electrophoresis, mass-spectrometry-based methodologies (SELDI, MALDI-TOF, LC-MS), antibody arrays, glycoproteins, autoantibodies, as reviewed (Ravelli, 2015; LeDu, 2013; Misek, 2011; Weigel, 2010). Comparisons between studies are made difficult due to differences in sample population, number, stage, and methodology. While biomarkers and even biomarker panels have been reported (Opstal, 2011; Shen, 2014; Chung, 2014; Liu, 2014), promising initial diagnostic performances often do not reproduce on subsequent diagnostic samples or larger cohorts, leaving these studies at the discovery stage.

More recently, “liquid biopsies” have generated great interest as potential source of biomarker discovery for cancer detection, monitoring and therapy response. The term globally refers to circulating tumor cells (CTCs) or nucleic acids in patient blood, including tumor or cell-free DNA (cfDNA, ctDNA), RNA from exosomes (exoRNA), circulating miRNA, (microRNA; Shen, 2014), mRNA and long non-coding RNAs (IncRNAs). The assumption is that circulating nucleic acids contain tumor-specific genetic aberrations that can be interrogated with current deep-sequencing technologies (Nik-Zainal, 2012; TCGAN, 2012) using a single blood sample (Kloten, 2013; Fackler, 2014). While they offer great potential, liquid biopsies still face technical challenges. cfDNA is shed by both healthy and tumor cells, and present in low amounts; there is lack of assay standardization, and variability among assay platforms, which remain costly so far. The accuracy and precision in detecting tumor specific genetic aberrations in patient serum or plasma is variable, and affected by the tremendous genetic heterogeneity of cancer. This limits the use of cfDNA in early detection and diagnosis (Coticchia, 2015; Buden, 2016). More promising results have been obtained in using ctDNA in monitoring metastatic breast cancer (Dawson, 2013), although cancer therapy induces mutations, making it difficult to differentiate mutations due to disease progression from those due to therapeutic intervention.

Therefore, an urgent need remains for a serum or blood-based, non-invasive, affordable, and easy-to-use in-vitro diagnostic assay (IVD) to complement and improve on current early detection methodologies.

With respect to monitoring breast cancer relapse in patients who already suffered a primary breast cancer, there is also a need for a simple, cost-effective and patient-friendly alternative to imaging. Indeed, improved early detection and treatments have translated into increased survival rates and impacted the number of breast cancer survivors: there are 3.6 million estimated breast cancer survivors in the US alone, and there will be 4.6 million by 2026 (Miller, 2016). Surveillance of breast cancer recurrence has thus become a significant priority in breast cancer management.

For those who have completed initial treatment for breast cancer (e.g. surgery, radiation, targeted therapy, and/or chemotherapy) the need for clinical follow-up is critical. Indeed breast cancer patients remain indefinitely at risk for local and/or systemic recurrence after their primary cancer, and carry a much higher 5 or 10 year breast cancer risk than the screening population (Shupe, 2014). While currently registries in the US do not routinely collect or report recurrence data (Mariotto, 2017), numerous studies have documented that breast cancer patients will eventually experience recurrence or develop distant metastasis after primary breast cancer beyond 10 and even over 20 years follow-up (Colleoni, 2016; Metzger-Filho, 2013). These findings emphasize the need for the development of cost-effective monitoring tools for breast cancer recurrence, which are presently lacking.

Surveillance of breast cancer recurrence relies on annual mammography screening, and regular physical examination and follow-up every 3-6 months for the first 3 years after primary therapy, then every 6-12 months for the next 2 years, and annually thereafter, according to the ASCO/ACS joint clinical practice guidelines (Runowicz, 2015). However a recent study found that only 50% of patients had undergone annual mammography, while 19% had undergone no imaging at all, indicating that a substantial proportion of patients does not follow surveillance guidelines (Ruddy, 2018). Thus, disease relapse is often detected in symptomatic patients upon imaging. In addition, because of the cost and the cumulative risk of radiation exposure, imaging cannot be recommended as a method to frequently monitor recurrence in breast cancer survivors.

Multigene signature tests mentioned above (e.g. Oncotype Dx) predict the risk of recurrence, and insofar they are one-time prognostic tests, yet they are not geared to monitor recurrence over a period of time as a surveillance procedure. Moreover, they mostly target patients with early stage ER+ primary tumors.

Therefore, biomarkers that can accurately monitor the emergence of a recurrence in asymptomatic patients prior to imaging would improve on current surveillance procedures. Detecting a biomarker change in a patient which is associated to recurrence, prior to appearance of symptoms and imaging results, would save the patients the toxic secondary effects of systemic therapies and increase their long-term survival while improving overall quality of life.

### SUMMARY OF THE INVENTION

The core of this invention is a non-naturally occurring monoclonal antibody, as well as compositions and methods related to said antibody that find application in the early detection of cancer, in the early detection of disease relapse and in monitoring therapy response.

By practicing the steps of the method of the present invention, the antibody presented herein enables the detection of protein cancer markers that alone, in combination with at least another biomarker, or collectively, provide a tool to discriminate with high probability patients with breast cancer, and preferably early stage breast cancer, thus informing the clinician on whether the patient requires further treatment, further exploratory procedures, active and regular follow-up, or can safely return at next recommended check-up.

The monoclonal antibody is capable to bind a collection of polypeptides disclosed herein at a binding site that displays similar features and amino acid functionalities to the binding site occurring in the other polypeptides. Common characteristics in the antibody binding site make up the antibody binding motif or epitope. Sequences with homology to the BF9 epitope or binding motif (e.g. mimotopes) are disclosed herein. The antibody may also contain markers or other functional entities allowing for the detection or localization of the markers, or of the monoclonal antibody, as well as for the detection of the binding of the antibody to the binding motif on the markers to form a complex at the epitope.

The protein markers recognized by the BF9 antibody are identified herein by their name, standard abbreviation and amino acid sequence, along with the nucleotide sequence encoding the polypeptide sequence of each marker. Indeed, encompassed in the compositions of the present inventions are the nucleotide sequences, as well as synthetic gene constructs, encoding the protein markers.

The detection also includes detecting non-natural variants of each of the foregoing in any assay format. The format for detection of the protein markers is not critical to utility of the invention and the markers, whether in the form of polypeptides or polynucleotides, and related species as defined herein can be detected by any existing technique known in the art for accurate identification of a polypeptide or polynucleotide sequence, or synthetic constructs based thereon, in a biological or patient sample, in addition to the immunoassay method described herein.

Because the protein markers are secreted from the cells of a human patient into a “biological fluid” or “patient test sample”, typically the blood or urine of the patient, the detection of the markers using conventional assay platforms for analysis of blood and urine is included within the invention. Identification of the markers also enables the detection of autoantibodies where present. The antibody described below for binding the markers may be used in any laboratory test format that uses a binding reaction between the polypeptide markers and the antibody to determine the presence of the markers in a biological sample.

The detection of the markers, the antibody, the genes or related species such as pre-RNA, mRNA, etc. can take place in an in vitro diagnostic kit for detection of cancer in a biological sample, or in a patient test sample, and in a large scale, high throughput format assay method or system for processing large numbers of samples.

The invention also includes methods for detecting the polynucleotide, pre-RNA, mRNA, or any species associated with transcription of the polynucleotides disclosed herein, or any species associated with the translation process yielding the markers. Also, the polypeptide markers may be transformed into a derivative or synthetic construct useful for detection or for creating novel or engineered antibodies for detection of the markers or a variant thereof. Also additional methods for using other antibodies, different from the one monoclonal antibody described herein, that are specific for the markers or variants thereof, are enabled.

The methods of the invention include measurement or detection of any component of the polypeptide markers including fragments, modifications, post translational modifications, truncations, or essentially any adequate representative sample of an amino acid sequence of which the polypeptide marker is comprised to determine the presence of the polypeptide in a sample. This includes using novel antibodies (polyclonal, monoclonal, Fab fragments, etc.) enabled by the description below to separate the markers described herein from a biological sample, such as a patient test sample in a test format wherein secreted proteins are identified. The monoclonal antibodies described herein can also be used in a diagnostic method to manufacture a new composition comprised of a complex of the novel monoclonal antibody and the markers. The methods also include distinguishing expression or secretion of the markers from other isoforms or variants of the markers, particularly where the detection events indicate the presence or progression of cancer or prognosis for, or response to treatment.

Specific uses of the methods described herein include detection of early cancer in the asymptomatic general population, detecting cancer in a suspect patient population having dense breast tissues or a high risk of developing cancer, tracking the status or progression of cancer in a patient, including the efficacy or success of a course of treatment over time by sequential measurement of the markers in a patient, preferably by secretion into a body fluid, but also including through measurement or analysis of gene expression or in tissue marker detection following a biopsy or imaging event. Similarly, by tracking the markers across a single patient over time, or through a population of patients at a fixed point in time or across numerous time periods, the efficacy of a new cancer treatment may be assessed. For example, where a new cancer therapeutic compound is under investigation, sequential measurements of the presence or quantity of the markers in a patient or a patient population provides an indication of the therapeutic utility of the clinical candidate.

The methods of the invention include detecting the markers described herein in a patient over time, after treatment for primary cancer, whereas changes in marker levels are indicative of disease relapse thus informing clinicians on the course of appropriate patient treatment, such as continue, stop or change therapy, pursue active surveillance with follow-up at regular intervals of time, confirm or exclude suspicious clinical status with further procedures, etc.

The invention also includes test devices, kits or methods for detecting the markers or related species, either alone or in combination with other markers, to assess the health or condition of a patient. The test can be in a panel format including the polypeptide and portions thereof, the polynucleotide, antibodies, or other entities or constructs described herein. The invention includes compositions specifically formulated and constructed for use as imaging agents to detect and localize the presence of the markers, or a form or variant thereof, in tissue or in an organ in the human body. Imaging or detection of the markers in vivo may include or be followed by biopsy, target radiation, or chemical therapy when or where the markers are detected.

The methods of the invention include the techniques and protocols specifically used for testing the asymptomatic general patient population for cancer, diagnosing a patient or groups of patients, and the practice of predictive medicine, including where specific populations of patients are identified and tested for the early development of cancer. These specific or pre-determined populations can be defined by age, sex, ethnic origin, prior disease, family history, genetic markers (such as Her-2, BRCA 1/2), exposure to toxins, carcinogens, or environmental or other cancer risk factors, or any event that places a patient in a defined or higher risk population.

The invention provides methods of determining or predicting effectiveness or response to a particular treatment, monitoring patient response to therapy, and methods of selecting a cancer treatment for an individual. For example, markers that are differentially expressed by cells (e.g., cancer cells) that are more or less responsive (sensitive) or resistant to a particular cancer treatment are useful for determining or predicting effectiveness or response to the treatment or for selecting a treatment for an individual.

Finally, the invention includes methods to detect cancer in an individual by measuring specific amounts of circulating or secreted markers in a biological or patient test fluid, such as in urine and serum, by immunological or other methods.

### DESCRIPTION OF FIGURES AND TABLES

FIG. 1: BF9 Mimotopes

Binding of individual phages, displaying 12-mer peptides BF9-12, BF9-15, and BF9-5 (SEQ ID NO: 46 to SEQ ID NO: 48), to monoclonal antibody BF9 by ELISA. Phage clones with the best binding activity are amplified and sequenced. The amino acid sequence of the 12-mers from the three best binders is provided. These sequences are referred to as mimotopes to indicate that they comprise amino acids displaying homology to the actual BF9 epitope and are used to screen for and identify the non-human monoclonal antibody described herein.

FIG. 2: Library immunoscreening of BF9 polypeptides

High-density protein macroarrays containing 27,648 individual E. coli expressed protein clones from a human fetal brain cDNA expression library were screened with the BF9 monoclonal antibody. Reactivity between expressed polypeptides and BF9 antibody was detected using anti-mouse IgG infrared 800 (Li-Cor) and signals were scanned with Odyssey imager at the 700/800 nm channels. Macroarrays were analyzed using scoring templates from the manufacturer to locate coordinates of duplicate spots, and then referring to a particular clone in the RZPD data base (https://www.ebi.ac.uk/arrayexpress/files/A-GEOD-15009/A-GEOD-15009.adf.txt).

FIG. 3: Differential expression of BF9 in breast cancer versus normal in 545 tissue samples by MPAT

Panel A. MPAT: protein extracts were prepared from frozen tissue biopsies of normal and normal adjacent tumor (N), benign (BN) and breast cancer (BC) patients according to Example 4, spotted in the same amount on the matrix protein array, and overlaid with BF9 monoclonal antibody. Immunodetection was via the fluorescence-based Li-cor Odyssey detection system as detailed in Example 3. Each row features 48 samples.

Sample breakdown is: 415 normal controls (N: 175 breast N and NAT, 240 colon N and NAT), 15 benign breast disease (BN, mostly fibroadenoma), and 115 BC, all ductal adenocarcinoma, including 59 early stage I+II (spotted in 1^{st}, and part of 2^{nd} row) and 56 late stage (spotted in last, and part of 2^{nd} row). Panel B. ROC curve analysis of BF9 in BC (n=115; all stages) versus non-BC (n=430) yielding 85% sensitivity at 80% specificity. MPAT data analysis was as described in Example 4.

Panel C. Scatter plot of all sample intensity values: spot intensity means of BC (3,219) versus non-BC (8,158) samples were compared using a t-test, and the difference was statistically significant (pvalue<0.0001). The 80^{th} percentile of normal, i.e. 4,750, is used as the cut off to discriminate BC versus non-BC. Statistical analysis was with GraphPad Prism (La Jolla, CA), as described in Example 10.

FIG. 4: Detection of BF9 marker secretion in cancer cell culture medium

Detection of secreted BF9 biomarkers in MCF7 and MRC5 serum free conditioned medium (SFCM) by direct ELISA. Plates were coated ON at 4° C. with equal amounts of SFCM proteins. After several PBS washes, plates were blocked in 1% BSA in PBS Tween 0.05% for 1 hr at 37° C., followed by addition of relevant BF9 monoclonal antibody for 1 hr. After several washes, a HRP-linked secondary antibody was added (1 hr 37° C.), followed by TMB substrate for 15-30 min at RT. The reaction was stopped with 1N H2SO4, and colorimetric detection was performed at 450 nm with a microplate reader.

FIG. 5: BF9 sandwich ELISA optimization

The BF9 sandwich ELISA uses a matched capture and detection monoclonal antibody pair. BF9 standard immunoassay protocol involves coating a 96-well immunoplate plate (Maxisorp) with capture monoclonal antibody in PBS, blocking unoccupied sites, incubating with surrogate antigen, binding captured BF9 markers with biotinylated detecting monoclonal antibody, amplifying detection with streptavidin linked horseradish peroxidase (HRP) and revealing colorimetric reaction with HRP substrate as measured at 450 nm by microplate reader. Serum-free cell culture medium (SFCM) from the breast cancer cell line MCF-7 is used as antigen source, and diluted in standard assay buffer. Antigen concentration is expressed as total protein concentration in the SFCM (μgE/ml).

Panel A. titration of capture and detection antibodies using different concentration of capture (0.5, 1, and 2 μg/ml) and detection antibody (0.5, 1, 2, and 3 μg/ml) to achieve optimal signal/noise ratio. OD450 nm values from wells with standard assay buffer only (“blank” or noise) and wells with a fixed amount of SFCM secreted BF9 antigens diluted in standard assay buffer are reported.

Panel B. Influence of various blocking agents to achieve optimal signal/noise ratio, used in various combinations either in the blocking buffer or in the detecting antibody diluent; optimal capture (2 μg/ml)/detect (1 μg/ml) antibody concentration was used. PF: protein free/PBS (Pierce); SB: SuperBlock (Pierce); CS: 1% casein/PBS (Pierce); BS: 1% BSA/PBS.

Panel C. Comparison of assay buffers B1 and B2 used as antigen diluent at various antigen concentrations. Column chart shows OD450 nm signals from blank wells with assay diluent only (0 μgE/ml total protein), and wells with various amount of MCF7-SFCM containing secreted BF9 antigens (1.9-17.1 μgE/ml total protein) diluted in either B1 or B2 buffer. Optimal capture (2 μg/ml)/detect (1 μg/ml) antibody concentrations and optimal blocking agent (PF/PBS) were used. B1 is standard assay buffer containing: 1% BSA in PBS-Tween 0.05%; B2 is serum optimized assay buffer containing: 1% BSA in PBS-Tween 0.05%, 10 mM EDTA, 5 mM DTT, 10 μg/ml of purified mouse lgG, 10 μg/ml of purified bovine IgG.

FIG. 6: BF9 Assay Characteristics

ELISA assay is described in detail in Example 9. Panel A. Typical antigen calibration curve. The sandwich ELISA assay uses matched capture and detection monoclonal antibody pair in standard buffer. Monoclonal antibodies were protein A/G purified from hybridoma culture supernatant or mouse ascites, and were used at the optimal concentration yielding the maximum signal to noise ratio with an acceptable non specific binding of OD<0.2. Serum-free cell culture medium (SFCM) from the breast cancer cell line MCF-7 is used as antigen source, and serially diluted in standard buffer. BF9 standard immunoassay protocol involves coating a 96-well plastic tray with capture monoclonal antibody, blocking unoccupied sites, incubating with surrogate antigen, binding captured polypeptides with biotinylated detecting monoclonal antibody, amplifying detection with streptavidin linked horseradish peroxidase (HRP) and revealing colorimetric reaction with HRP substrate as measured at 450 nm by microplate reader. Each data point is in triplicate, and BF9 polypeptide concentration equivalent is interpolated from the standard curve generated by using the 4 parameter logistic method.

Panel B. Analytical dilution recovery. A known amount of BF9 surrogate antigen source is spiked into assay diluent supplemented with 10% of a normal human serum pool containing undetectable levels of said antigen. The sample is then serially diluted in standard buffer, and BF9 antigen levels measured in quadruplicate samples. The average measured concentration of quadruplicate values (SD shown on graph; derived from the standard curve) is plotted against the expected concentration. % recovery is the ratio between the expected antigen concentration and the average measured concentration. Regression analysis yields a slope of 0.971 and R²=0.999, indicating linear dilution in the range of concentrations tested. % CV=SD/mean*100.

Panel C. Analytical spiking recovery. Normal human serum pool diluted 1:10 in assay diluent and containing undetectable levels of antigen, is spiked, in quadruplicate, with 5 amounts of BF9 surrogate antigen source, at concentrations ranging from 1.07 to 17.1 μgE/ml. Recovered antigen concentration is measured and interpolated using the BF9 calibration curve. Quadruplicate values of each measured concentration are averaged. % recovery and % CV are calculated as in panel B above and are within acceptable limits.

Panel D. Inter-assay reproducibility. The spiking recovery experiment described in panel B above using 5 different concentrations of surrogate antigen source was repeated in triplicate and in 3 independent days to measure inter-assay variance. % recovery and % CV of 9 measurements are reported and within acceptable limits.

FIG. 7: BF9 Diagnostic Performance

BF9 marker levels were measured in duplicate in 179 serum samples with the BF9 ELISA assay. The sample set comprised: 71 BC patients, 32 BN (12 proliferative and 20 non-proliferative fibrocystic changes, FCC), and 76NL controls. BF9 marker concentrations were derived from a standard curve as described in Example 9. The median biomarker concentration values in each group were compared to determine whether differences were statistically significant (p<0.05). A. Scatter plot of all sample intensity values illustrates BF9 marker distribution and median (indicated by a line) differences among groups. B-F. ROC curves were calculated to evaluate BF9 diagnostic performance in 5 major comparisons, by determining SE, SP, area under the curve (AUC), and confidence interval (CI).

FIG. 8: Summary data on BF9 diagnostic performance

This figure summarizes BF9 SE, SP and CI at various cutoffs (μgE/ml), for 5 different comparisons.

FIG. 9: BF9 for monitoring breast cancer recurrence

Panel A. Number of recurrent patients with positive test (>cutoff; SE %) detected by BF9, CA15-3 (>30 U/ML) and CEA (>5 ng/ml) using standard cutoff values, either alone or in combination. BF9 cutoff values of >17.1 or >21.2 μgE/ml imply a 71% SP and 80% SP respectively in a BC vs NL comparison (FIG. 7).

Panel B. Serial assessment of tumor burden in mice. MCF-7 cells (˜5×10⁶ in PBS) were subcutaneously injected in each of 4 nude immunodeficient mice, and xenografts allowed to develop. Serum was serially obtained from mice at the indicated times and assayed in duplicate to measure BF9 levels. Top: Representative images of tumor growth in mouse #3 at week 1, 2, 3 post-injection. Bottom: Time course of BF9 levels for each mouse; levels at each time point are the mean±SD (indicated by bars) of duplicate measures from 2 independent experiments. Baseline BF9 levels prior to injection (week 0) were subtracted from each time point value.

FIG. 10: Monitoring patient disease from diagnosis to recurrence

Individual patient charts illustrate patient follow-up using the BF9 assay. Serial serum samples from 15 patients after a primary breast cancer were measured and test results were plotted versus days of follow-up. Any available indication of disease relapse and treatment event (chemotherapy, radiation, hormonal treatment, metastasis at different sites) is indicated. Abbreviations: AWD (alive with disease); DOD (dead of disease); NED (no evidence of disease).

FIG. 11: Immunodetection of secreted BF9 markers in urine

Panel A: Diagram illustrating the spot location of 47 urine samples from patients with: pancreatic cancer (n=5; 4 stage II and 1 unknown), colon cancer (n=10; all stages I or II), colon inflammatory diseases (n=5), colon benign conditions (n=4), prostate cancer (n=13; all stage II except D9 and D10 of stage III) and normal controls (10). Note that cancer stages I and II are defined as “early”, while stages Ill and IV are defined as “late”.

Panel B: Total urine proteins after centrifugation to remove debris, were spotted on the MPAT membrane in a blind experiment, in three different conditions: as is, or diluted 1:2 or 1:10 in Tris-Triton buffer as indicated. Protein samples were then incubated with BF9 monoclonal antibody and immunodetected as described in Example 13

Table 1: Patient clinicopathological data in Clinical Sample Set #2

Breast cancer patient characteristics are listed. The relationship between clinicopathological parameters and serum biomarker levels was examined. The median BF9 biomarker concentration values in each group were compared to determine whether differences were statistically significant (p<0.05).

Table 2: Patient clinicopathological data in Clinical Sample Set #4

The breakdown of breast cancer patients used in the pilot longitudinal study is provided for each clinicopathological feature analyzed: age, menopausal and nodal status, histological type, stage grade, ER/PR and HER2 status, and time to recurrence are indicated. Note: * 1 patient incurred LRR and DR; several patients presented over time with multiple metastatic sites. Abbreviations: Average (AVG); standard deviation (SD); NED (no evidence of disease); LRR (loco-regional recurrence); DR (distant recurrence); ER (estrogen receptor); PR (progesterone receptor).

Table 3: Urine clinical samples

Composition of the clinical sample set used to detect the presence of polypeptides or fragments thereof bearing a BF9 binding motif in urine of cancer patients, benign and normal controls, as illustrated in FIG. 11. Details on disease stage and inflammatory conditions are provided in FIG. 11 and its legend. E: early stage cancer (stages I and II); L: late stage cancer (stages III and IV); U: unknown.

### DETAILED DESCRIPTION OF THE INVENTION

The present invention relates to composition and methods using a novel monoclonal antibody designated BF9 that recognizes biomarkers whose polypeptide sequence is listed herein (SEQ ID NO: 1 to SEQ ID NO: 48) which find application in the early detection of cancer, in the early detection of disease relapse, and in the monitoring of therapy response. These markers, which have in common and are defined by a BF9binding motif, are collectively and interchangeably referred to herein as “BF44 markers”, “BF9 biomarkers”, “BF9 polypeptides” or “BF9 proteins”. The use herein of the plural “markers”, “biomarkers”, “polypeptides” or “proteins” refers to any single species as well as to any combination thereof (of 2, 3, 4, or more). Specifically, any combination of 2, 3, 4, 5, 6, 7, 8, 9, 10, 11, 12, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 proteins is contemplated by the present invention. The present invention discloses that BF9markers are differentially expressed (over-expressed) in individuals with cancer as compared to individuals without cancer (individuals without cancer are interchangeably referred to herein as “normal”, “control”, or “healthy” individuals). The present invention revolves around the use of BF9 monoclonal antibody that provides a tool to discriminate with high accuracy patients with cancer, and preferably patients with early stage breast cancer.

BF9 may be used in a variety of clinical indications for cancer, including, but not limited to, detection of cancer (such as in an asymptomatic individual or population or in a high-risk individual or population), characterizing cancer (e.g., determining cancer type, sub-type, or stage) such as distinguishing between breast cancer subtypes (or otherwise facilitating histopathology), determining whether a lesion is a benign lesion or a malignant tumor (including using BF9 for imaging), cancer prognosis, monitoring cancer progression or remission, monitoring for cancer recurrence, monitoring metastasis, selecting treatment, monitoring response to a therapeutic agent or other treatment, stratification of patients for MRI or computed tomography (CT) screening (e.g., identifying those patients at greater risk of cancer and thereby most likely to benefit from enhanced screening, thus increasing the positive predictive value of any parallel screening method), combining testing of BF9 markers with supplemental biomedical parameters and patient clinical information, such as those listed in Table 1, or such as toxin exposure, smoking history, BRCA-1 or -2 presence, or any of the existing markers noted below, or with tumor or nodule size, tumor morphology, etc. (such as to provide an assay with increased diagnostic performance compared to another testing technique alone or in combination with BF9), facilitating the diagnosis of a biological sample as malignant or benign, facilitating clinical decision making once a cancer is observed by margins, or of biopsy if the sample is deemed medium to high risk, and facilitating decisions regarding clinical follow-up (e.g., whether to implement repeat detection of this or another marker, imaging, biopsy, or other measure).

BF9 markers may be quantified when diagnosing cancer such that a high or low abundance level in an individual who is not known to have cancer may indicate that a threshold amount present in a sample from the individual correlates to cancer at a specific stage, thereby enabling early detection of cancer at an early stage of the disease when treatment is most effective, i.e. perhaps before the cancer is detectable by other techniques or before other symptoms appear. An increase in the abundance of BF9 markers may be indicative of cancer progression, e.g., a tumor or abnormal tissue is growing and/or metastasizing (and thus a poor prognosis), whereas a decrease in the abundance of BF9 markers may be indicative of cancer remission, e.g., a tumor is shrinking (and thus a good prognosis). Similarly, an increase in the abundance of BF9 markers during the course of cancer treatment may indicate that the cancer is progressing and therefore indicate that the treatment is ineffective, whereas a decrease in the abundance of BF9 markers during the course of cancer treatment may be indicative of cancer remission and therefore indicate that the treatment is working successfully. Additionally, an increase or decrease in the abundance of BF9 markers after an individual has apparently been cured of cancer may be indicative of cancer recurrence or metastasis. Detection of “differential” expression, or variation from a “normal” expression level, can also be used for another purpose described herein.

Detection of BF9 markers may be particularly useful following, or in conjunction with cancer treatment, such as to evaluate the success of the treatment or to monitor cancer remission, recurrence, and/or progression (including metastasis) following treatment. Cancer treatment may include, for example, administration of a therapeutic agent to a patient, surgery (e.g., surgical resection of at least a portion of abnormal tissue or a tumor), radiation therapy, or any other type of cancer treatment used in the art, and any combination of these treatments.

BF9 monoclonal antibody and antibodies to BF9 markers may also be used in imaging tests. For example, an imaging agent can be coupled to BF9 which can be used to aid in cancer screening or diagnosis, to monitor disease recurrence, progression/remission or metastasis, to plan surgery, biopsy, or radiation therapy, or to monitor response to therapy, among other uses. The monoclonal antibodies disclosed herein are formulated to enhance stability, reduce immunogenicity and enhance plasma half-life, pH-range stability and other desirable pharmacological parameters by techniques known in the art.

As used herein the term “antibody” refers to a polyclonal, monoclonal, recombinant antibody, full-size molecule or antibody fragment thereof, including but not limited to Fab “”, scFv, single chain variable fragment, affibodies, diabodies, or any other antibody fragment, or any other recombinant version of conventional or combinatorial antibody, as well as any single or double chained binding agents comprised of a variety of known structures, including another molecule or biologically compatible tag that facilitates detection of the antibody while retaining the ability of the antibody to recognize the relevant epitope or common characteristics of the BF9 binding motif to a sufficient extent for detection to occur.

Unless specified, the term “antibody” is used interchangeably herein to refer to any of the above species. The novel compositions are comprised of non-naturally occurring species of monoclonal antibodies capable of binding BF9 markers. Methods of the invention include use of both naturally occurring and synthetic variants of BF9 antibody. Thus, “antibodies” include antibodies produced in vitro, as well as antibodies generated in vivo by injection of BF9 markers or polynucleotides encoding BF9 markers in a mammal capable of mounting a sufficient immune response to yield high titre lgG antibodies. Methods to produce polyclonal, monoclonal, recombinant antibodies and fragment thereof are known to the skilled in the art (Coligan et al., Current Protocols in Immunology, Wiley Intersciences; Kohler et al. Nature 256:495-497, 1975; Phage display of peptides and proteins-A laboratory manual, Kay B. B., Winter J. & McCafferty J., Eds, Academic Press, 1996).

The term “monoclonal antibody”, as used herein, refers to a novel antibody obtained from a population of substantially homogeneous antibodies, i.e., the individual antibodies comprising the population are substantially identical except for naturally occurring mutations present in minor amounts. Monoclonal antibodies are highly specific and are typically directed against a single epitope and variants thereof as described below, in contrast to polyclonal antibody preparations which typically include different antibodies directed against different determinants (epitopes). In addition to specificity, the monoclonal antibody against BF9 markers described herein is substantially homogenous and is produced by an available hybridoma. The modifier “monoclonal” indicates that the antibody exists in a substantially homogeneous population of antibodies, but is not to be construed as requiring production of the antibody by any particular method.

An “isolated” or “purified” antibody is one that has been identified and separated and/or recovered from a component of the environment in which it is produced. Contaminant components of its production environment are materials that would interfere with diagnostic or therapeutic uses for the antibody, and may include enzymes, hormones, and other proteinaceous or nonproteinaceous solutes. In exemplary embodiments, the antibody can be purified as measurable by any of at least three different methods: 1) to greater than 95% by weight of antibody as determined by the Lowry method, preferably more than 99% by weight; 2) to a degree sufficient to obtain at least 15 residues of N-terminal or internal amino acid sequence by use of a spinning cup sequenator or 3) to homogeneity by SDS-PAGE under reducing or non-reducing conditions using Coomassie blue or silver stain. Isolated antibody can include an antibody in situ within recombinant cells since at least one component of the antibody's natural environment will not be present. Ordinarily, however, an isolated antibody can be prepared by at least one purification step.

A “transformed antibody” is a binding protein produced in a (host) species other than the species of the antigen (target) to which the antibody specifically binds. The transformed antibody typically has chemical or structural signatures characteristic of the host that do not exist in the target species. An example is a “transformed” BF9 antibody to the human BF9 proteins produced in a bacterial species such as an E Coli or in a mammalian species such a CHO cell having glycosylation or other chemically distinct signatures compared to a BF9 antibody existing or produced in a mammal or vertebrate.

“Antibody specificity” refers to an antibody that has a stronger binding affinity for BF9 polypeptides from a first individual species than it has for a homologue of BF9 from a second species. Typically, a BF9 antibody “binds specifically” to a human BF9 antigens (e.g., has a binding affinity (Kd) value of no more than about 1×10⁻⁷ M, preferably no more than about 1×10⁻⁸ M, and most preferably no more than about 1 ×10⁻⁹ M) but has a binding affinity for a homologue of the antigen from a second individual species at least about 50-fold, or at least about 500-fold, or at least about 1000-fold, weaker than its binding affinity for the human BF9 polypeptides.

An antibody “selectively” or “specifically” binds the BF9 marker proteins when the antibody binds the marker proteins and does not significantly bind to unrelated proteins. An antibody can still be considered to selectively or specifically bind a marker protein even if it also binds to other proteins that are not substantially homologous with the marker protein as long as such proteins share substantial homology with a fragment or domain of the marker protein epitope. Antibody binding to the marker protein is still selective and “specific” despite some degree of cross-reactivity to other antigens.

The term “epitope” is used to refer to the amino acid sequence within the BF9 marker polypeptides recognized by the BF9 antibody disclosed herein. The term “epitope”, “antigenic determinant”, “structural domain”, “antibody target” or “binding motif” are interchangeably used to indicate the amino acid sequence, whether in isolated form or embedded in a polypeptide sequence or fragment and derivative thereof, which is recognized by the BF9 antibody. Epitopic determinants can be active surface groupings of molecules such as amino acids or sugar side chains and may have specific three-dimensional structural characteristics or charge characteristics. Preferably, we will refer herein to the common characteristics of the binding motif, recognized by the BF9 monoclonal antibody and shared by the BF9 protein markers, to exemplify the well known notion in the art of antibody-antigen interactions whereby an antibody can bind to different polypeptide sequences sharing epitope homology, as described above. Said homology includes amino acid changes, preferably involving conservative amino acid substitutions, yet also including any amino acid substitution that maintains binding functionality, such as permutations, deletions, or insertions. As a result, alterations to the sequence of the epitope may exist as long as the BF9 antibody retains binding specificity as determined by the ability of the BF9 antibody to bind the BF9 markers at the altered epitope to form a complex in such a way that the binding event is detectable. Such altered sequences of the epitope are also referred to as “mimotopes” to indicate that they comprise sequences displaying homology, yet not identical, to the epitope, thus mimicking epitope functionality.

Therefore encompassed herein are binding agents other than an antibody as defined herein, such as but not limited to aptamer, peptide nucleic acid (PNA) as well as a polymer, solid support or chemical scaffold displaying anti-BF9 mimotopes or binding motif enabling the detection of the BF9 compositions.

The terms “natural polynucleotide”, or “natural nucleotide sequence”, are used interchangeably herein and may include naturally occurring DNA sequences or downstream transcripts such as pre-RNA. The “natural polynucleotide” described herein is DNA, including genomic DNA, double or single-stranded, whether coding or non-coding strands, or RNA, including heteronuclear RNA, messenger RNA (mRNA), or any other form of RNA, such as small, anti-sense, interfering or silencing RNA whose expression correlates to the presence or expression of BF9 markers in vivo or in a biological sample.

A “synthetic polynucleotide” or “polynucleotide construct” may contain introns, 5′ and 3′ non-coding sequences, 5′ and 3′ transcriptional regulatory sequences, such as promoters, enhancers, polyadenylation signals, or translational control elements not present in the natural polynucleotides encoding the polypeptide markers as expressed in a human patient. The synthetic polynucleotides may include “natural polypeptide” sequences for BF9 markers that are manufactured to include DNA constructs to facilitate expression or regulation or that encode for leader or secretory sequences at the level of the polypeptides, or for active or inactive pro-proteins that are later processed into active or inactive shorter polypeptides. The assembled synthetic constructs are constructed and oriented to facilitate expression of the natural polypeptide in a non-natural environment.

The synthetic polynucleotides described herein include engineered splice variants and non-naturally-occurring allelic variants, and any non-natural variants encoding the biomarkers of the present invention with a different nucleotide sequence due to the degeneracy of the genetic code. Variants encode fragments, analogs and derivatives of the markers, and may include deletion, substitution, addition or insertion variants created by design even if duplicated by unusual and rare phenomena including those created. The synthetic polynucleotides encompassed by the claims include any length of said polynucleotide sequences, whether 5′ terminal, 3′ terminal or internal and transformed into entities chemically suited for use in a diagnostic platform.

Synthetic polynucleotides of the present invention, including DNA constructs can be manufactured using standard molecular biology techniques and the sequence information described herein (Sambrook et al., 1989, Molecular Cloning: A Laboratory Manual, 2^{nd} ed., Cold Spring Harbor Laboratory Press, Cold Spring Harbor, NY).

The BF9 protein markers are substantially free of cellular material or free of chemical precursors or other chemicals. BF9 proteins can be purified to homogeneity or other degrees of purity. The level of purification can be based on the intended use. The primary consideration is that the preparations allow for the desired function of the proteins, even if in the presence of considerable amounts of other components.

To determine the percent identity of two amino acid sequences i.e. a reference and a test sequence such as any naturally occurring BF9 polynucleotide and another sequence such as a synthetic sequence, the sequences can be aligned for optimal comparison purposes (e.g., gaps can be introduced in one or both of a first and a second amino acid or polynucleotide sequence for optimal alignment and non-homologous sequences can be disregarded for comparison purposes). In an exemplary embodiment, at least 30%, 40%, 50%, 60%, 70%; 80% or 90%, 91%, 92%, 93%, 94%, 95%, 96%, 97%, 98%, 99% or 100% of the length of a reference sequence can be aligned for comparison purposes. A test sequence may also be tested for equivalent reactivity, including specific reaction of a test polypeptide or polypeptide encoded by a test polynucleotide, with a BF9 antibody, particularly a novel antibody binding at the binding motif described herein. The amino acid residues or nucleotides at corresponding amino acid positions or nucleotide positions are compared and relative functionality analyzed by techniques known in the art. When a position in the first sequence is occupied by the same amino acid residue or nucleotide as the corresponding position in the second sequence, then the molecules are identical at that position (as used herein, amino acid or nucleic acid “identity” is equivalent to amino acid or nucleic acid “homology”). The percent identity between the two sequences is a function of the number of identical positions shared by the sequences, taking into account the number of gaps, and the length of each gap, that are introduced for optimal alignment of the two sequences.

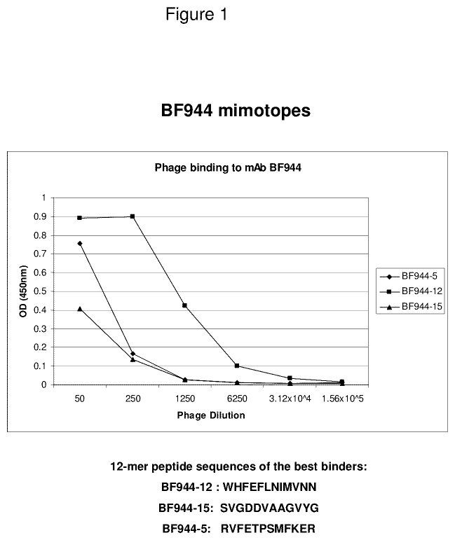

The monoclonal antibody disclosed herein is monoclonal antibody BF9 and is preferably identified by its ability to recognize a collection of markers, namely the BF9 polypeptides listed herein (SEQ ID NO: 1 to SEQ ID NO: 48) by binding a motif that shares common characteristics among the polypeptides, as exemplified by the mimotopes listed herein (SEQ ID NO: 46 to SEQ ID NO: 48; FIG. 1). The antibodies encompassed by the present invention include all antibodies, as defined above, that are capable of binding (having specific binding affinity) to BF9 markers, both naturally occurring in humans and synthesized by known chemical or biological techniques, and preferably those polypeptide variants containing the binding motif.

The reactivity of monoclonal antibody BF9 of the present invention is specifically targeted to the BF9 polypeptides (SEQ ID NO: 1 to SEQ ID NO: 48) encoded by the corresponding polynucleotides (SEQ ID NO: 49 to SEQ ID NO: 96) OR to polypeptides (SEQ ID NO: 1 to SEQ ID NO: 48) encoding the BF9 markers, as long as such species harbor the BF9 binding motif facilitating use of the marker in any embodiment of the present invention. The specific novel monoclonal antibody BF9 disclosed herein may also be reactive against proteins or fragments thereof not listed herein that share substantial similarity in antigenic determinants or structural domains (substantially similar epitopes, e.g. mimotopes). Indeed it is well-established and known to those skilled in the art that protein families performing similar cellular functions share functional domains in the form of highly conserved amino acid sequence motifs, which become the “functional” signature of those given proteins and their variants (polymerase, kinase, protease, etc.). Hence, other related target polypeptides may share amino acid motifs or functional domains with BF9 markers listed herein.

A “biological sample(s)” as referred to herein is a quantity of tissue, or body fluid or other material from human patient or normal controls, and comprises tissues and/or biological fluids containing a polypeptide expressed by the patient. Tissue samples include, but are not limited to fresh or frozen normal or diseased tissues (including normal, tumor adjacent tissues), particularly cancer tissues, such as derived from a tumor biopsy cell line (lysate or intact) extracts, including the extracts of the MPAT assay described below, or any other preparation that may be processed for advantageous use in the methods or kits of the invention, and including from different organ sites, different histological types of cancer, and different stages (early, advanced, metastatic), but also tissues from benign and/or inflammatory conditions at a given organ site. A “patient test sample(s)” includes any body fluid, obtained by a non-invasive sampling method, used for detection of secreted proteins, including but not limited to, urine (precipitated or unprecipitated), plasma, serum, blood, saliva, sputum, nipple aspirate fluid, any lavages (such as but not restricted to ductal lavages) or bronchio-alveolar lavages. The term “patient” refers to a human previously diagnosed with disease or an asymptomatic person screened for disease.

In preferred embodiments, the biological samples examined are matched normal and tumor tissues derived from the same patient including, normal adjacent tumor samples derived from the same or different cancer patients. Samples may include primary tumor or metastasis, early or late stages of cancer, from stage I to stage IV, as well as benign tumors and inflammatory conditions. For the purpose of the present invention, biological samples referred herein may also include mammalian cell cultures, preferably cancer cell lines, as well as microdissected cell types from normal or disease tissue samples, or from a given subcellular compartment.

### BF9 Polypeptides

The present invention includes compositions comprising and methods using polypeptides having the amino acid sequences (SEQ ID NO: 1 to SEQ ID NO: 48) that are recognized by monoclonal antibody BF9 of the present invention whereby their detection, alone, in combination of 2, 3, 4, or more, or collectively, enable the discrimination with high accuracy of patients with breast cancer thus finding application in the early detection of cancer, in the early detection of disease relapse, and in the monitoring of therapy response. As specified above, any combination of 2, 3, 4, 5, 6, 7, 8, 9, 10, 11, 12, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 proteins is contemplated by the present invention. The protein identity of BF9 markers recognized by the BF9 monoclonal antibody can be determined by a variety of approaches known in the art, including but not limited to epitope mapping by phage display, human expression library immunoscreening, and proteomics (immunocapture mass spectrometry), as described in Examples 1-2. Epitope mapping by phage display is based on the screening of a library of phages displaying a population of 12-mer peptides on the capsid, such population comprising random combinatorial sequences of amino acids that are not naturally-occurring in a living organism. Best 12-mer binders to BF9 monoclonal antibody are selected, as described in detail in Example 1. Insofar this method results in identifying mimotopes having homology to the BF9 epitope (FIG. 1). BLAST search of the NCBI protein databases (blast.ncbi.nlm.nih.gov) yields the identity of the polypeptides recognized by BF9. Specifically, the sequence of the best binding peptide is entered to retrieve proteins featuring a sequence with highest homology to the queried peptide, as described in detail in Example 2.

BF9 marker polypeptides recognized by BF9 monoclonal antibody may be identified through other methods, such as human expression library immunoscreening. In another embodiment, we screened high-density protein macroarrays containing 27,648 individual E. coli expressed protein clones from a human fetal brain cDNA expression library with the BF9 monoclonal antibody (Bussow et al., 1998), as described in Example 2. BF9 reactivity with specific proteins expressed in situ from bacteria spotted on the macroarrays was analyzed, scored and the corresponding clone sequences were identified through the RZPD database (https://www.ebi.ac.uk/arrayexpress/files/A-GEOD-15009/A-GEOD-15009.adf.txt).

BF9 marker polyppetides recognized by the BF9 monoclonal antibody and disclosed herein are: Putative uncharacterized protein ASB16-AS1 (ASB16-AS1, SEQ ID NO: 1); Spectrin alpha chain, non erythrocytic 1 (SPTAN1, SEQ ID NO: 2); High Mobility Group Protein HMGI-C (HMGA2, SEQ ID NO: 3); Histone H1.2 (HIST1H1C, SEQ ID NO: 4); Serine/arginine-rich splicing factor 4 (SRSF4, SEQ ID NO: 5); Cytosolic Fe-S cluster assembly factor (NUBP2, SEQ ID NO: 6); Glutamine amidotransferase-like class 1 domain-containing protein 3b, mitochondrial (GATD3B, SEQ ID NO: 7); Core histone macro-H2A.2 (H2AFY2, SEQ ID NO: 8); Zinc finger protein 214 (ZNF214, SEQ ID NO: 9); Uncharacterized protein C6orf132 (C6orf132, SEQ ID NO: 10); Zinc finger protein 629 (ZNF629, SEQ ID NO: 11); Coiled-coil domain-containing protein 185 (CCDC185, SEQ ID NO: 12); Protein FAM161A (FAM161A, SEQ ID NO: 13); Calcium-binding mitochondrial carrier protein ScaMC-1 (SLC25A24, SEQ ID NO: 14); Zinc finger protein 112 (ZNF112, SEQ ID NO: 15); Zinc finger protein 133 (ZNF133, SEQ ID NO: 16); Protein prune homolog 2 (PRUNE2, SEQ ID NO: 17); Serine/arginine repetitive matrix protein 3 (SRRM3, SEQ ID NO: 18); Selenoprotein O (SELENOO, SEQ ID NO: 19); Zinc finger protein 536 (ZNF 536, SEQ ID NO: 20); Ring finger protein 17 (RNF17, SEQ ID NO: 21); Prame family member 17 (PRAMEF17, SEQ ID NO: 22); NGFI-A-binding protein 1 (NAB1, SEQ ID NO: 23); Glycoprotein integral membrane protein 1 (GINM1, SEQ ID NO: 24); Neuroligin-4 X-linked (NLGN4X, SEQ ID NO: 25); Putative deoxyribonuclease, (TATDN1, SEQ ID NO: 26); Calcineurin B homologous protein 3 (TESC, SEQ ID NO: 27); Sperm protein associated with the nucleus on the X chromosome (A/C/D) (SPANX, SEQ ID NO: 28); CUE domain-containing protein 2 (CUEDC2, SEQ ID NO: 29); Nuclease-sensitive element-binding protein 1 (YBX1, SEQ ID NO: 30); Protein S100-A8 (S100A8, SEQ ID NO: 31); Alpha enolase (ENO1, SEQ ID NO: 32); Calmodulin-like protein 5 (CALML5, SEQ ID NO: 33); High Mobility Group Protein HMG-I/HMG-Y (HMGA1, SEQ ID NO: 34); Iron-sulfur protein NUBPL (NUBPL, SEQ ID NO: 35); Ubiquilin 4 (UBQLN4, SEQ ID NO: 36), Ras-related protein RAB-11B (RAB11B, SEQ ID NO: 37); Vesicle trafficking protein SEC22b (SEC22B, SEQ ID NO: 38); Proline-rich protein 11 (PRR11, SEQ ID NO: 39); Myc target protein (MYCT1, SEQ ID NO: 40); Centrin-2 (CETN2, SEQ ID NO: 41); NGFI-A-binding protein 2, (NAB2, SEQ ID NO: 42); Lysine-specific histone demethylase 1A (KDM1A, SEQ ID NO: 43); Vacuolar protein sorting-associated protein 8 homolog (VPS8, SEQ ID NO: 44); Polycystic kidney and hepatic disease 1-like protein 1 (PKHD1L1, SEQ ID NO: 45).

The present invention comprises the BF9 amino acid sequences listed (SEQ ID NO: 1 to SEQ ID NO: 48) as well as a population of polypeptides having related or identical sequences to SEQ ID NO: 1 to SEQ ID NO: 48 such as isoforms, fragments, variants and derivatives thereof, and related polypeptide variants sharing homology with BF9 binding.

The compositions of the BF9 polypeptides include any non-naturally occurring species manufactured from the synthetic polynucleotide sequences claimed herein by conventional and non-conventional mechanisms, such as frameshift, either occasional or programmed, internal initiation, or non Watson-Crick codon-anticodon pairing events at the translation level. These and other mechanisms may lead to the production of hybrid or synthetic polypeptides of BF9, for example carrying amino acid motifs of one reading frame and/or amino acid motifs expressed from another reading frame. Such hybrid proteins carrying multiple amino acid domains may consequently be regulated according to as many different functional domains as featured in the hybrid polypeptide. Synthetic or hybrid polypeptides may retain substantially the same biological function or activity as the relevant biomarker while partially differing in any degree from the natural amino acid sequence.

Non-naturally occurring variants of the BF9 proteins can readily be generated using recombinant techniques. Such variants include, but are not limited to, deletions, additions, and substitutions in the amino acid sequence of the BF9 protein markers. For example, one class of substitutions is conserved amino acid substitutions. Such substitutions are those that substitute a given amino acid in a BF9 polypeptide by another amino acid of like characteristics. Typically seen as conservative substitutions are the replacements, one for another, among the aliphatic amino acids Ala, Val, Leu, and lle; interchange of the hydroxyl residues Ser and Thr; exchange of the acidic residues Asp and Glu; substitution between the amide residues Asn and Gln; exchange of the basic residues Lys and Arg; and replacements among the aromatic residues Phe and Tyr. Guidance concerning which amino acid changes are likely to be phenotypically silent is found in Bowie et al., Science 247:1306-1310 (1990). Such amino acid substitutions underlie the concept of a BF9 common binding motif and correlate BF9 mimotopes (Figure1) to BF9 polypeptide sequences listed herein.

Included herein are variants of the BF9 marker polypeptide sequences obtained by recombinant or gene synthesis techniques using sequences from the relevant as well as from different expressed proteins, such as mimicking the products of gene fusions naturally occurring in cancer, as long as those fusion constructs preserve the BF9 binding motif. Indeed structural chromosome rearrangements result in the exchange of coding or regulatory DNA sequences between genes. Many such gene fusions are mutation drivers in cancer and have been associated to tumorigenesis (Mertens et al. Nat Rev Cancer 15:371-381, (2015)).

Amino acids that are essential for function can be identified by methods known in the art, such as site-directed mutagenesis or alanine-scanning mutagenesis (Cunningham et al., Science 244:1081-1085 (1989)). The latter procedure introduces single alanine mutations at every residue in the molecule. The resulting mutant molecules are then tested for biological activity or in assays such as in vitro proliferative activity. Sites that are critical for partner/substrate binding can also be determined by structural analysis such as crystallization, nuclear magnetic resonance, or photoaffinity labeling (Smith et al., J Mat. Biol. 224:899-904 (1992); de Vos et al., Science 255:306-312 (1992)).

Compositions, variants or fragments of naturally occurring BF9 marker polypeptides useful in the methods of the invention typically comprise at least about 5, 6, 8, 10, 12, 14, 16, 18, 20 or more contiguous amino acid residues of the marker. Such fragments can be chosen based on the ability to retain one or more of the biological activities of the marker or can be chosen for the ability to perform a function, e.g., bind a substrate or act as an immunogen. Particularly important fragments are biologically active fragments, such as peptides that are, for example, about 8 or more amino acids in length. Such fragments can include a domain or motif found in BF9 markers, e.g., an active site, a transmembrane domain, or a binding domain, preferably the BF9 binding motif. Further, possible fragments include, but are not limited to, soluble peptide fragments and fragments containing immunogenic structures. Domains and functional sites can readily be identified, for example, by computer programs well known and readily available to those of skill in the art (e.g., PROSITE analysis).

Variants of the BF9 marker polypeptides may also be comprised of non-naturally occurring modifications to the BF9 polypeptides including, but not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, sialylation gamma carboxylation, glycosylation, GPI anchor formation, hydroxylation, iodination, methylation, myristoylation, oxidation, proteolytic processing, phosphorylation, prenylation, racemization, selenoylation, sulfation, tRNA-mediated addition of amino acids to proteins such as arginylation, and ubiquitination.

Such modifications are well known to those of skill in the art and have been described in the scientific literature. Several particularly common modifications, glycosylation, lipid attachment, sulfation, gamma-carboxylation of glutamic acid residues, hydroxylation and ADP-ribosylation, for instance, are described in most basic texts, such as Proteins-Structure and Molecular Properties, 2nd Ed., T. E. Creighton, W. H. Freeman and Company, New York (1993). Many detailed reviews are available on this individual, such as by Wold (Posttranslational Covalent Modification of Proteins, H. C. Johnson, Ed., Academic Press, New York 1-12 (1983)); Seifter et al. (Meth. Enzymol. 182:626-646 (1990)); and Rattan et al. (Aim. N.Y. Acad. Sci. 663:48-62 (1992)).

BF9 marker polypeptides encompassed by the present invention may include fusion to a marker sequence supplied by an expression vector and enabling purification of the polypeptide of the present invention, such as hexa-histidine tag, glutathione-S-transferase, hemagglutinin, luciferase, beta-galactosidase, and the like. The polypeptides may also include polypeptides, in full or in part, modified by any form of post-translational modification, such as phosphorylation, acylation, methylation, ubiquitination, etc., conjugation or covalent linkage to lipids, polysaccharides and the like. These polypeptides further include full-length mature folded proteins, or fragments thereof, either derived by internal initiation, early termination, degradation, or post-translational processing. Non-naturally occurring polypeptide variants of BF9 marker polypeptides may be distinguished from naturally occurring forms by several parameters including characterizing unique sequence content, conjugation with other chemical species, alterations in glycosylation or other chemical signatures including sialylation, any altered structural or chemical composition resulting from expression in non-mammalian expression systems or organisms, altered folding characteristics from non-mammalian expression or processing including measured variances in folding structure caused by separation on a column or other purification or processing techniques.

The source of the polypeptides include a natural polypeptide purified from a biological mixture such as that of a protein extract from human specimens, or a recombinant polypeptide generated by various methods known in the art as described herein, or a purely synthetic polypeptide. Whether recombinant or synthetic, naturally-occurring BF9 polypeptides or BF9 variants can be generated based on the sequence information disclosed herein (SEQ ID NO: 1 to SEQ ID NO: 48).

Variant polypeptides of BF9 marker polypeptides also include isolated antigenic determinants, epitope sequences, or other structural protein domains, produced by different methods known those skilled in the art, including but not limited to: direct peptide synthesis using conventional solid-phase techniques (Merrifield, 1963), direct gene synthesis, in vitro run-off transcription from vectors carrying bacteriophage promoters, high-throughput cell-free translation systems (Sawasaki, 2002), and by recombinant techniques aiming at the expression and purification of recombinant proteins or protein fragments from bacterial, yeast, insect, or mammalian expression vectors that are commercially available and known to those in the art.

Variant polypeptides of BF9 markers can also be purified from cells that express them, purified from cells that have been altered to express them (recombinant), or synthesized using known protein synthesis methods (e.g., Sambrook et al., Molecular Cloning: A Laboratory Manual. 3rd. ed., Cold Spring Harbor Laboratory Press, Cold Spring Harbor, N.Y., (2001)). For example, a natural or synthetic polynucleotide encoding a BF9 marker protein is integrated into an expression vector, the expression vector introduced into a host cell, and the non-naturally occurring BF9 polypeptide variant expressed in the host cell. The polypeptide variant can then be isolated from the cells by an appropriate purification scheme using standard protein purification techniques.

### BF9 Polynucleotides

The present invention includes polynucleotide species which encode BF9 polypeptides (SEQ ID NO: 1 to SEQ ID NO: 48) that are recognized by monoclonal antibody BF9 of the present invention and that alone, in combination with at least another, or collectively, enable the discrimination with high probability of patients with breast cancer thus finding application in the early detection of cancer, in the early detection of disease relapse, and in the monitoring of therapy response.

### BF9 Genes

Exemplary BF9 nucleic acid molecules of the invention consist essentially of, or comprise nucleotide sequences that encode BF9 marker proteins of the invention, allelic variants thereof, and orthologs or paralogs thereof for example. As used herein, a synthetic polynucleotide bears chemical signatures resulting from defined differences between the synthetic entity and the nucleic acid sequence of the natural polynucleotide. Preferably, the synthetic polynucleotide is free of sequences which naturally flank the nucleic acid (i.e. sequences located at the 5′ and 3′ ends of the nucleic acid) in the genomic DNA of the organism from which the natural polynucleotide is derived. The synthetic polynucleotide typically includes synthetic flanking sequences, particularly contiguous protein-encoding sequences and protein-encoding sequences within the same gene but separated by introns in the genomic sequence, and flanking nucleotide sequences that contain regulatory elements. The primary consideration is that the nucleic acid is distinguishable from the naturally occurring sequence by engineered or manufactured manipulations described herein including recombinant expression, the design and preparation of probes and primers, and other features such as a non-naturally occurring transcript/cDNA molecule, or synthetic polynucleotide produced by recombinant technique, or chemical synthesis.

A synthetic polynucleotide can be comprised of the naturally occurring polynucleotide and fused to other coding or regulatory sequences and still be considered synthetic. Synthetic polynucleotides can include heterologous nucleotide sequences, such as heterologous nucleotide sequences that are fused to a nucleic acid molecule by recombinant techniques. For example, recombinant DNA molecules contained in a vector are considered synthetic. Further examples of synthetic DNA molecules include recombinant DNA molecules maintained in heterologous host cells, or purified (partially or substantially) non-naturally-occurring DNA molecules in solution. Synthetic pre-RNA or RNA molecules include in vivo or in vitro RNA transcripts of synthetic DNA molecules as long as the species is not naturally occurring, but may include species produced by unusual or rare phenomenon. Synthetic nucleic acid molecules further include such variant molecules produced synthetically.

Synthetic polynucleotides encode a mature protein plus additional amino or carboxyl-terminal amino acids, or amino acids interior to the mature protein (when the mature form has more than one peptide chain, for instance). Such sequences may play a role in processing of a protein from precursor to a mature form, facilitate protein trafficking, prolong or shorten protein half-life, or facilitate manipulation of a protein for assay or production, among other things. As generally is the case in situ, additional amino acids may be processed away from the mature protein by cellular enzymes.

Synthetic nucleic acid molecules include, but are not limited to, sequences encoding a BF9 polypeptide variant alone, sequences encoding a mature protein with additional coding sequences (such as a leader or secretory sequence (e.g., a pre-pro or pro-protein sequence), and sequences encoding a mature protein (with or without additional coding sequences) plus additional non-coding sequences (e.g., introns and non-coding 5′ and 3′ sequences such as transcribed but non-translated sequences that play a role in transcription, mRNA processing (including splicing and polyadenylation signals), ribosome binding, and/or stability of mRNA). In addition, synthetic polynucleotides can encode a BF9 polypeptide variants that facilitate purification.

Synthetic polynucleotides including cDNA and genomic DNA obtained by cloning or produced by chemical synthetic techniques or by a combination, can be double-stranded or single-stranded. Single-stranded nucleic acid can be the coding strand (sense strand) or the non-coding strand (anti-sense strand).

Synthetic polynucleotides are non-naturally occurring variants made by random or targeted mutagenesis techniques, including those applied to isolated nucleic acid molecules, cells, or organisms. Accordingly, nucleic acid molecule variants can contain nucleotide substitutions, and sequence deletions, inversions, and/or insertions can occur in either or both the coding and non-coding regions, and variations can produce conservative and/or non-conservative amino acid substitutions.

A fragment of a synthetic polynucleotide typically comprises a contiguous nucleotide sequence at least 8, 10, 12, 15, 16, 18, 20, 22, 25, 30, 40, 50, 100, 150, 200, 250, 500 (or any other number in-between) or more nucleotides in length and encodes epitope bearing regions or binding motif of the encoded BF9 polypeptides particularly for separation of the protein from related isoforms or variants as DNA probes and primers.

A probe/primer typically comprises a substantially purified oligonucleotide or oligonucleotide pair. An oligonucleotide typically comprises a nucleotide sequence that hybridizes under stringent conditions to at least about 8, 10, 12, 14, 15, 16, 17, 18, 19, 20, 21, 22, 23, 24, 25, 30, 40, 50 (or any other number in-between) or more contiguous nucleotides.

As used herein, the term “hybridizes under stringent conditions” is intended to describe conditions for hybridization and washing under which nucleotide sequences encoding a protein at least 60-70% homologous to each other typically remain hybridized to each other. The conditions can be such that sequences at least about 60%, at least about 70%, or at least about 80% or more homologous to each other typically remain hybridized to each other. Such stringent conditions are known to those skilled in the art and can be found in, for example, Current Protocols in Molecular Biology, John Wiley & Sons, N.Y. (1989-2006). One example of stringent hybridization conditions is hybridization in 6× sodium chloride/sodium citrate (SSC) at about 45° C., followed by one or more washes in 0.2× SSC, 0.1% SDS at 50-65° C.

### Biomarker Detection in Cancer by MPAT

Monoclonal antibody BF9 enables the detection of BF9 marker polypeptides, through specific binding of the antibody to said polypeptides carrying a BF9 binding motif, and enables measurement of their expression in biological samples, and patient test samples, particularly detection in protein samples from normal and disease human specimens, where the disease is cancer, or from cancer tissue or cell lines, thus correlating expression of BF9 marker proteins with the presence of cancer.

### Matrix Protein Array Technology

Assessment of differential expression of BF9 marker poylpeptides includes immunodetection, and specifically using monoclonal antibody BF9 in a process named the Matrix Protein Array Technology (MPAT) as presented below and described in detail in Example 3.

The MPAT is a multiplex protein array immunoassay that simultaneously analyzes multiple biological samples. In essence, the MPAT is an immunoassay linked to a data acquisition and imaging system, whereby the same matrix of samples is simultaneously interrogated by an antibody. Then, a secondary antibody, preferably linked to a chemiluminescent probe or fluorescent dye, is used to visualize antigen-antibody reaction for each sample, and a scanned image of all reactions is produced with an imaging system, processed and analyzed to yield the simultaneous examination in multiplex format of the relative expression levels of a number of proteins of interest.

The solid support of the matrix protein array is preferably nitrocellulose or glass, yet can be made of a variety of materials that include, but are not limited to: plastic, polystyrene, nylon, teflon, ceramic, fiber optic and semiconductor materials. The solid support of the matrix protein array is composed of different physical areas that can be referred to as wells, compartments, surfaces, and the like, distinctly separated from each other. These physical areas can adopt a variety of surfaces and volumes, and the support can accommodate from 1 or 2 to more than 10,000 compartments, depending on the needs, leading to an extremely versatile tool. Each compartment may contain biological samples from the same type, different types, the same species, different species, the same physiological condition, different physiological conditions or any combination of the above arrayed on the solid support. Each compartment is overlaid with any identifier, preferably an antibody, as selected.

It is understood by those skilled in the art that the device and methodology described herein as MPAT allows all kind of combination of biological samples, number of samples, conditions of samples, size of compartment of the matrix protein arrays, type of identifiers, or any permutation of the above. Furthermore, while in the present invention, the MPAT methodology described below is applied to human biological samples, it is understood to the skilled in the art that the MPAT is widely applicable to protein samples derived from any organism, including animal, bacterium, yeast, fungus, or plant.

In its simplest format, the MPAT is composed of 96 chambers although other formats can be used depending on the number of antibodies to assay and the number of samples to screen. In a given MPAT experiment, the same matrix of protein extracts from different biological samples (e.g. clinical specimens or cancer cell lines as described below) is printed in each chamber, and each chamber is assayed with a distinct individual antibody. Each individual compartment is then overlaid with a distinct antibody and processed for the detection of antigen-antibody complexes. This format allows direct comparison between multiple samples (including normal and diseased samples) under the same conditions, preventing day-to-day experimental variability, as it is often observed in other proteomic studies (Diamandis EP, Analysis of serum proteomic patterns for early cancer diagnosis: drawing attention to potential problems, J Natl Cancer Inst 96:353-356, 2004a; Diamandis EP, Mass Spectrometry as a diagnostic and cancer biomarker discovery tool, Mol Cell Proteomics 3:367-378, 2004b; Ransohoff D F, Rules of evidence for cancer molecular-marker discovery and validation, Nature Rev Cancer 4:309-314, 2004) or DNA microarray experiments (Dudoit S, Gentleman R C, Quackenbusch J, Open source software for the analysis of microarray data, Biotechniques 34: S45-S51, 2003; Gabor Miklos GL and Maleszka R, Microarray reality checks in the context of a complex disease, Nature Biotechnol 22:615-618, 2004).

In the immunodetection analysis detailed in Example 3 and described in a number of preferred embodiments herein, the detection and isolation of the markers disclosed herein from within a complex biological mixture (i.e. antibody-antigen complexes) is preferably performed by way of a chemiluminescent reaction, although other protocols based on other labeling and detection systems, such as alkaline-phosphatase, biotin-streptavidine, and fluorescence can also be successfully used within the scope of the present invention. Antigen-antibody signals are captured by a charge-coupled device (CCD-camera) or a Li-cor-Odyssey infrared imaging system, processed and quantified by specialized software, as described in Example 3.

### BF9 Detects Differential Expression in Cancer Versus Normal

BF9 monoclonal antibody enables the detection of differential protein expression in patient tissue samples (specifically in protein extracts thereof) including cancer, normal, and benign.

We used the MPAT platform and up to 545 protein extracts prepared from tissue biopsies of 115 breast cancer (BC) patients (early and late stage) and 430 controls, including 15 benign (BN) breast disease, and 415 normal (N) and normal adjacent tissues (NAT); from 175 breast and 240 colon samples). Preparation of tissue protein extracts is described in Example 4. Signal intensity observed in the MPAT is directly related to the extent of antigen-antibody interaction. As evidenced, BF9 detected over-expression of markers in BC tissues, in early as well as in late stages of disease (FIG. 3A). Differential expression in BC versus N was quantified and found statistically significant (p<0.0001; FIG. 3BC). ROC curve analysis established sensitivity and specificity of 85% SE at 80% SP and 90% area under the curve (AUC), demonstrating BF9 excellent discriminatory power in this sample set.

These data demonstrate the presence in patient tissues of BF9 binding polypeptides whose differential expression is associated to cancer, and the utility of BF9 compositions of the present invention as markers for cancer detection.

### Detection by Immunohistochemistry

Immunohistochemistry (IHC) is a commonly practiced in vitro diagnostic procedure to determine normal vs. disease in a patient tissue biopsy. The patient tissue biopsy is first formalin-fixed and paraffin-embedded, then sectioned at 3-5 micrometer thick and mounted on treated microscope glass slides to enhance tissue adherence. Slides are stained with a relevant antibody against a cellular marker in a procedure described in details in Example 6. Tissue microarrays (TMA) can also be used instead of individual slides to analyze the reactivity of an antibody, or marker expression, in a large number of patient samples to establish marker prevalence in a biological sample of the patient.

Encompassed in this invention is performing IHC analysis with monoclonal antibody BF9 and tissue slides featuring cancer tissues and normal controls, preferably adjacent normal controls (NAT above). Typically an immunostaining procedure comprises the use of anti-mouse IgG biotinylated secondary antibody followed by streptavidin linked to horseradish peroxidase, finally followed by the addition of AEC substrate. Other immunostain procedures are contemplated: for example, protocols based on different labeling and detection systems, such as alkaline-phosphatase, biotin-streptavidine, or fluorophores can also be successfully performed within the scope of the present invention. Furthermore, while most tissues undergo pre-treatment to inactivate endogenous peroxidase, if peroxidase-based staining is used, pre-treatment is not necessary when using fluorescence-based imaging system.

Knowledge of biomarker localization is important in diagnostic applications. Proteins have different localization within the cell depending on their function, including secreted (such as growth factors, hormones, neuropeptides), present on the cell surface (such as glycoproteins, glycolipids and receptors) intracellular (within the cytosol, or in particular sub-cell compartments, such as the nucleus, the Golgi, or the endoplasmic reticulum). BF9 marker polypeptides can be localized to cellular structures via the use of BF9 antibody by a variety of techniques known to those skilled in the art, which can be performed on mammalian cell suspension or adherent cells, and which are described in (Current Protocols in Immunology, Wiley Interscience, John E. Colligan et al.), such as but not limited to, immunohistochemistry, immunfluorescence (IF) using FACScan (FACS), flow cytometry (FC) and indirect IF, but also electron microscopy and other imaging techniques providing localization to subcellular structures.

### Detection by Western Blot

Encompassed herein is Western blot analysis, as described in details in Example 7, using BF9 antibody to characterize BF9 markers in protein extracts from human cancer cell lines, protein extracts derived from matched or unmatched normal and tumor tissue samples from cancer patients, or from any other biological sample as defined herein above. Protein extracts are separated by gel electrophoresis, transferred to nitrocellulose and probed with BF9 antibody to visualize the corresponding antigen protein bands.

### Detection of BF9 Marker Secretion in Cancer Cell Culture Medium

In another embodiment of the invention, BF9 antibody enabled the detection of BF9 markers in serum free cell culture medium (SFCM) of a breast cancer cell line (MCF7) as biofluid surrogate (prepared as detailed in Example 8), by direct ELISA (FIG. 4). BF9 markers, or fragments thereof displaying BF9 binding motif were indeed detected in this medium, as opposed to SFCM from the negative control (MRC5, a normal embryonic fibroblast cell line). This result demonstrates that BF9 markers are secreted in the culture supernatant of the MCF7 cancer cell line, suggesting that they may be further secreted in patient body fluids.

### Detection of BF9 Marker Secretion in Biological Fluids: patient serum

The present invention demonstrates that BF9 monoclonal antibody detects BF9 marker polypeptides secreted in the supernatant of a cancer cell line, thereby suggesting secretion of BF9 markers in patient body fluids. Examples of patient test samples include, but are not limited to, blood, lymph, serum, plasma, urine, gynecological fluids and smears, bronchio-alveolar lavages, sputum, nipple aspirate fluids, etc. In many instances, such samples are associated with the detection of diseases and conditions at specific organ sites, e.g. bronchio-alveolar lavages for asthma, lung cancer and lung diseases, nipple aspirate fluids for breast cancer, urine sediments after digital rectal examination for prostate cancer, etc. Among body fluids, serum and urine are particularly important, as they represent an informative biological material not requiring invasive procedures.

We were further able to detect binding of BF9 antibody to BF9 polypeptides in a small number of serum samples from breast cancer patients, versus normal controls by direct ELISA. The fact that BF9 markers are found secreted not only in SFCM, but also in patient serum, eventually prompted the development of a serum-based sandwich ELISA assay.

### BF9 Serum-Based Sandwich ELISA Assay Development

BF9 ELISA assay is described in detail in Example 9, while features of ELISA assay optimization in patient serum are described in FIG. 5 its legend. Each assay is a typical sandwich ELISA assay using matched capture and detection monoclonal antibody pair to detect polypeptides of the CRC polypeptide panel in patient serum. A standard immunoassay protocol involves coating a 96-well plastic tray with capture monoclonal antibody, blocking unoccupied sites, incubating with antigen source (patient serum, other patient biological fluid, or see below), binding captured CRC related polypeptides with biotinylated detecting monoclonal antibody, amplifying detection signal with streptavidin linked horseradish peroxidase (HRP) and revealing the resulting colorimetric reaction with HRP substrate (3,3′,5,5′-Tetramethylbenzidine, TMB) by measuring absorbance (OD) at 450 nm with a microplate reader.

Thus the actual assay output is an optical density: the higher the optical density, the more antibody-antigen interaction is detected in the assay, and the higher the levels of BF9 polypeptides in patient serum. To translate the measurement of an optical density, into measurement of marker levels in a patient, a standard calibration curve is used in the art. A standard calibration curve for BF9 is shown in FIG. 6A and described in more detail in Example 9. Briefly, known amount of antigens are measured in the assay thus enabling plotting OD values (on the y axis) against concentrations or antigen levels (on the x axis). Calibration curves thus enable a clinical laboratory running the assay or a software incorporated into the assay to transform raw assay data into biomarker levels in patient serum.

However to further determine whether a patient health status is “diseased”, “normal” or maybe “suspicious”, marker levels in a patient need to be compared to a reference value. Reference values are typically obtained in a population of healthy individuals of same age group and gender than the disease of interest (e.g. female of >50 years old in breast cancer screening). Reference values may vary depending on the clinical application of the assay, such as reference values to detect early stage cancer may differ from reference values to monitor occurrence of disease relapse in a population of patients who has already suffered a primary cancer and is under follow-up, or such as reference values in a population of patients under treatment who are being monitored with respect to therapy efficacy.

The reference value thus establishes the threshold (also known as cutoff) above which the test score is considered “positive” thus defining the person as diseased, or as suspicious needing further procedures, or at risk of carrying the disease etc. Accordingly diagnostic performance is defined by the terms “sensitivity” (proportion of truly diseased subjects in the screened population or test who are identified as positive or “diseased” by the test), and “specificity” (proportion of persons without the disease who have scores below the cutoff on the test) with respect to a given reference value or cutoff, preferably expressed with respect to a given clinical application. All such parameters, but not limited to, come into play when a diagnostic assay measures biomarkers in a patient.

To ensure that the BF9 immunoassay was suitable for measuring clinical serum samples, recovery, linearity, and reproducibility of the assay were established. Assay characteristics were determined as described in FIG. 6. Both dilution and spiking recovery were measured to ensure that BF9 assay yields an analytical antigen recovery within the acceptable range of 80-120% (FIG. 6BC), and shows linearity of dilution (R²˜1; FIG. 6B), indicating minimal serum interference. Finally, we measured BF9 intra and inter-assay reproducibility. Intra-assay reproducibility was measured in quadruplicate samples, yielding a percent coefficient of variation (% CV) between 5.5and 11.9 (FIG. 6B) and between 10.8 and 14.9 (FIG. 6C). The % CV <15% in both experiments is within the accepted limits. Inter-assay reproducibility was measured in triplicate samples and on 3 independent days, yielding a % CV between 6.1 and 10.6 (FIG. 6D), a range which is also within the accepted limits.

### BF9 Diagnostic Performance in the Early Detection of Breast Cancer

In preliminary studies we showed that BF9 serum-based immunoassay could discriminate all the breast cancer patients (n=14) from healthy controls (n=13; Clinical Sample Set #1) in a small sample set. The discrimination between breast cancer and control group was statistically significant and prompted us to establish in the present study the diagnostic performance of the BF9 assay in a larger clinical sample set with an emphasis on early breast cancer stage. All Clinical Sample Sets are described in Example 12.

The 179 serum sample set (Clinical Sample Set #2) was designed to test the diagnostic performance of BF9 in the early detection of breast cancer, and insofar comprised early breast cancer cases (n=71), as well as proliferative and non-proliferative benign breast conditions (n=32), and normal controls (n=76). Table 1 summarizes the clinicopathological features of the breast cancer samples. BF9 levels were measured with BF9 ELISA assay in duplicate in all samples diluted 1:10 in assay diluent, and BF9 marker concentrations were derived from the biomarker standard curve as described using 4-parameter logistic method (Example 9). Statistical analysis (Example 10) of two or multiple group comparisons was carried out based on the median value of the biomarker concentration (μgE/ml) in each group to determine whether differences between groups were statistically significant. The scatter plot representation of all serum BF9 measurements in this clinical sample set illustrates biomarker distribution in each group and shows the striking differences in biomarker median level among normal and benign groups on the one hand, and the breast cancer group on the other hand (FIG. 7A).

The major result from this study is the ability of the BF9 assay to discriminate breast cancer (n=71) from normal controls (n=76), as anticipated from prior data, with 87.3% SE at 90.8% SP (AUC=0.941; p-value<0.0001) demonstrating excellent discriminatory power, at a cutoff of 36.3 μgE/ml in this sample set (FIG. 7B, FIG. 8). Given that the 71 BC cases comprise 68 TNM stage I/II and one ductal carcinoma in situ (stage TO; DCIS), the data show the potential utility of the BF9 assay as a test for screening and diagnosis breast cancer, including in the early stages of disease.

The diagnostic performance of BF9 in this sample set was evaluated by ROC curve analysis in different group comparisons. When 20 non-proliferative benign fibrocystic changes (FCC) are included with the normal controls (n=76), the discrimination between breast cancer and non-breast cancer (n=96) yields 87.3% SE at 88.5% SP (AUC=0.931; p-value<0.0001; FIG. 7C, FIG. 8). When 32 benign conditions, comprising 20 non-proliferative FCC and 12 proliferative cases, are included in the controls, BF9 discriminates breast cancer (n=71) from non-breast cancer (normal and benign; n=108) with 87.3% SE at 85.2% SP (AUC=0.909; p-value<0.0001; FIG. 7D, FIG. 8). The inclusion of benign in the control group slightly reduces BF9 SP, at the same cutoff, without affecting SE (FIG. 8). Finally, when only the benign group is considered, BF9 is still able to discriminate breast cancer (n=71) versus the non-proliferative benign group (n=20; FCC), at the same cutoff, with 87.3% SE at 80% SP (AUC=0.894; p-value<0.0001; FIG. 7E, FIG. 8), while the comparison versus all benign (n=32) yields 87.3% SE at a decreased 71.9% SP (AUC=0.834; p-value<0.0001; FIG. 7F, FIG. 8). Overall, at the cutoff of 36.3 μgE/ml and in this sample set, BF9could correctly identify 90.8% of healthy subjects, 87.3% of breast cancer cases, 80% of non proliferative benign FCC and 60% of the proliferative benign subgroup (FIG. 8).

Overall these data (FIG. 8) demonstrate the second major result in this study: that the BF9 assay can distinguish breast cancer and early breast cancer not only from normal controls, but also from benign conditions of the breast, and particularly from non-proliferative FCC. Although BF9 specificity decreases from 90.8% towards normal controls, to 88.5% towards normal and FCC, to 85.2% towards normal and all benign, and to 80% towards FCC only, this result suggests the potential clinical application of BF9 assay as adjunct to mammography in discriminating normal and non-proliferative benign controls from breast cancer, including early breast cancer stage, preferably in the clinical application of breast cancer screening.

Statistical analysis shows that the median levels of BF9 markers measured by the assay in the normal (n=76; 8.9 μgEq/ml), FCC (n=20; 11.2 μgEq/ml), proliferative benign (n=12; 32.8 μgEq/ml), and ef-the benign group (n=32; 16.5 μgEq/ml) are not significantly different between each group. However, as described above, the median biomarker levels in each control group is statistically significantly different when compared to that of the breast cancer group (FIG. 7A-F), except for the proliferative benign group. Indeed, in this sample set, no statistically significant difference is found in median marker concentration in the proliferative benign group (n=12; 11 FAD and 1 ADH; 32.8 μgE/ml) versus the breast cancer group (n=71; 101.2 μgE/ml).

### Clinicopathological Features

Finally, the association between serum levels of BF9 markers detected by the assay and the clinicopathological characteristics of the breast cancer patients was investigated. No significant correlation was found (p>0.05) between biomarker level and age, tumor size, nodal status, TNM stage, grade (Table 1). With respect to subtypes, no statistically significant difference was found between triple negative breast cancer patients (n=15) and HR+/HER2− (whether ER, PR or both), as summarized in Table1. However, data suggested that HER2− patients show higher levels than HER2+ patients.

### Comparison Among Different Sites

Both cases and controls in this study were obtained from different sites, one in the US (site A) and two abroad (sites B and C). Indeed, cases were obtained from sites A and B, while control samples were procured from sites A, B and C (Clinical Samples Set #2; Example 12). Therefore we investigated whether any biomarker level differences might be due to sample origin. First, we found that there were no statistically significant differences (p>0.9999) between the median biomarker levels in the control samples form sites A (n=33; 7.2 μgEq/ml), B (n=24; 9.4 μgEq/ml) and C (n=19; 10.9 μgEq/ml). Then we compared the ROC curves in the breast cancer versus normal comparison using the site A only set (33 N; 14 BC; 78% stage I+II BC) and the site B only set (24 N; 57 BC; 100% stage I+II BC). Seemingly identical AUC values of 0.9351 and 0.9342respectively were obtained with the same 95% confidence interval (0.9059<CI<0.9766) and p<0.0001, indicating the same diagnostic performance of the BF9 assay in this comparison and in the two sets (data not shown). We thus confidently conclude that the biomarker levels observed in this study, as well as the differences in biomarker median values between groups are not dependent on sample source differences.

### Association with Age

Since cases and controls (Clinical Sample Set #2; Example 12) were not matched for age, we investigated whether the age means of the normal and breast cancer groups were statistically different. First, we found that there were no statistically significant differences between the age mean in the control samples from site A (n=33; 44+14), site B (n=24; 43+18) and site C (n=19; 44+10) (adjusted p-value>0.9999 for all comparisons), similarly to what we found above for the biomarker level and the different geographic sources. Then, we assessed that the age means of breast cancer samples procured from site A (n=8, as 6 samples were missing age information; 59+11) and the site B (n=57; 60+12) were not statistically significantly different (adjusted p-value>0.9999). And finally, we compared the age means of the breast cancer group (n=65; 60+12) to the normal control group (n=76; 44+14) and found that the difference was statistically significant (p<0.0001). Although no statistical differences were found between the age mean of the normal (n=33) and BC (n=8) group from site A (adjusted p-value=0.3163), overall the control population was slightly younger. Therefore we further investigated whether the biomarker values differed by age.

As mentioned above there was no correlation between biomarker level and age in the breast cancer group (Table1). Indeed no statistically significant difference was found (p-value=0.1434) between the median biomarker concentration in the <50 age group (n=19; 127 μgEq/ml) and the >50 age group (n=46; 89.3 μgEq/ml). Likewise, no statistically significant difference was found (p-value=0.3404) between the median biomarker concentration in the <50 age group (n=49; 8.6 μgEq/ml) and the >50 age group (n=27; 9.1 μgEq/ml) in the normal control group. Thus biomarker levels do not vary with age, either in cases or in controls, and while the two groups display age differences, said differences are independent from diagnostic performance.

In conclusion, the BF9 assay enables: i) the discrimination of early breast cancer stages, and ii) the discrimination of non-proliferative benign and normal controls from breast cancer. These findings prompt two major possible applications for BF9assay as adjunct to mammography: i) as second line screening after suspicious mammography in average breasts, dense breasts, and high risk population to decrease the number and cost of additional procedures, and ii) as pre-screening test in women 40-49 years of age at average risk of breast cancer, in the non-compliant population, and by extension in the developing world, and even in the young and dense breast population.

### Correlation Between Secretion of BF9 Polypeptides and Tumor Burden

To demonstrate correlation between tumor burden and secretion of BF9 marker polypeptides in the serum of breast cancer patients, we applied the BF9 assay to the serum of xenografted mice, as animal tumor models. Nude immunodeficient mice were subcutaneously injected with MFC-7 cells (shown to secrete polypeptides binding BF9, FIG. 9), and allowed to develop xenografts, as described in Example 11. Tumor growth was visually inspected and monitored, as shown in FIG. 9B (top). Mouse serum was tested with BF9 ELISA assay before injection, and on a weekly basis thereafter. BF9 biomarker levels in serum increased with time from the baseline level prior to injection, along with tumor growth, in all four xenografts although to different extent (143-fold in mouse #4 to 1226-fold in mouse #1 by week 2; FIG. 9B bottom). Mice #1 and #3, with the fastest growing tumors, were sacrificed due to excessive tumor growth at weeks 2 and 3 respectively. The experiment progressed for mouse #2 until tumor necrosis occurred, with concomitant drop in serum biomarker levels. Mouse #4 had no apparent tumor at week 1, then tumor grew, albeit at a much slower rate than in the other xenografts. This study overall shows a reasonable correlation between increasing tumor burden and increasing marker levels, while tumor necrosis is accompanied by markers' decline.

### Use of the BF9 Assay in Monitoring

The correlation between tumor burden and serum levels of BF9 marker polypeptides in mouse xenografts (FIG. 9B), and the fact that the BF9 assay detects with high SE/SP early stage primary breast cancer (FIGS. 7 and 8), prompted us to test whether the BF9 biomarkers recognized by the BF9 antibody might serve, alone, in combination with at least another biomarker of the present invention, or in combination with other known tumor markers (see below) as early indicators of disease relapse. We explored whether the BF9 assay could be applied to monitoring breast cancer recurrence. To that end we applied the BF9 assay to in a population of 416 breast cancer patients with recurrent and metastatic disease and annotated CA15-3/CEA levels measured at hospital visit (Advia Centaur, Siemens; Clinical Sample Set #3; Example 12) obtained at a clinical center. Thus, the performance of the BF9 assay in detecting recurrent breast cancer could be compared to that of traditional serum markers at their standard cutoff values (30 U/ml and 5 ng/ml were used for CA15-3 and CEA respectively; Stieber, 2015). Cutoff values of 17.1 μgE/ml and 21.2 μgE/ml were considered for the BF9 assay, as they yield a 71.1% SP and 80.3% SP respectively in a breast cancer versus NL comparison (FIG. 8), representing acceptable SP values in this clinical setting. The data show that the BF9 assay alone detects more breast cancer cases than CA15-3 (24.5%) and CEA (16.3%) alone or in combination (30%), reaching 76.2% SE and 68.3% SE with a cutoff of 17.1 or 21.2 μgE/ml respectively (FIG. 9A). Further, both CA15-3, and CEA to a lesser extent, complement BF9 biomarkers, thus enabling the panel combination to reach 81.7% SE and 76.2% SE at the cutoff of 17.1 and 21.2 μgE/ml respectively. These data strongly suggest that the BF9 assay may be superior to traditional serum biomarkers in monitoring breast cancer recurrence.

### Pilot Longitudinal Study

The preliminary studies described above show that marker polypeptides recognized by BF9 monoclonal antibody have serum levels that correlate with tumor burden and disease progression in xenografted mice, and that the BF9 assay significantly detects breast cancer recurrences in a cross-sectional study, complementing and outperforming CA15-3/CEA. To further evaluate whether the BF9 assay might serve as an early indicator of disease relapse, we evaluated BF9biomarkers in a set of serial samples collected from patients treated and followed up after a primary breast cancer and retrospectively obtained (Clinical Sample Set #4; Example 12). Patient clinicopathological features are summarized in Table 2. BF9 markers were measured in duplicate and in a blind fashion. Then, upon sample unblinding, biomarker levels were matched with well annotated clinical information and analyzed (FIG. 10).

As summarized in Table 2, overall a total of fifteen (n=15) patients diagnosed with invasive breast cancer, mostly ductal adenocarcinoma, were followed-up after diagnosis and surgery for a minimum of 18 months to a maximum of 9.9 years. Four (n=4) patients remained NED for a minimum of 18 months to a maximum of 7.9 years of follow-up. Eleven (n=11) patients incurred disease relapse with a time to recurrence ranging from 128 to 2250 days, and a mean average of 1074 days (2.9 years) post-surgery. Recurrences included 1 loco-regional (LRR) and 11 distant (DR) events with metastatic sites at lung, bone and liver and gastro-intestinal tract. Overall, the set comprised 70 serum samples, ranging from a minimum of 3 to a maximum of 9 samples per patient. Specifically 21 samples were clinically identified as NED, and 26 clinically identified as disease, including pre-surgery, loco-regional recurrence, metastasis at various organ sites, disease progression (PD) and diagnosis of a second and third breast cancer. Finally 23 intermediate collection time points were associated to patient treatment information (type, regimen). Most samples were also associated to CA15-3 levels measured during patient follow-up.

FIG. 10 features individual patient charts where BF9 biomarker levels are plotted versus time of follow-up and any available indication of disease relapse and treatment events are provided. Patient 11 displays the profile expected for a marker predictive of recurrent disease (high at pre-surgery and at metastasis, and lower at the intermediate time point), while patient 15 displays the marker profile of a disease free patient (high at pre-surgery, decreasing at post-surgery until the patient is considered clinically NED).

Patients 4, 7, 8 display BF9 biomarker levels around or below cutoff (36 μgE/ml) at baseline, e.g. initiation of FEC adjuvant therapy after surgery (FIG. 10). Of the 8 time points associated with clinical disease (metastasis at liver, lung or bone sites, disease progression, 2nd or 3rd breast cancer, death) 7 display significantly high levels of BF9 markers, whether above the cutoff, above the baseline or above the level of the preceding time point. In patient 8 disease progression at the lung is clinically detected at day 413: interestingly, BF9 levels in the 4 intermediate collection time points preceding day 413 are consistently above baseline, from 146.8 μgE/ml at day 57 to 191.9 μgE/ml at day 413. This result suggests that monitoring with BF9 assay could have detected disease progression with a lead time of one year (356 days) before symptom appearance. In this group of patients, 4/4 disease free time points correlated with normal levels of BF9 markers, and 7/8 time points with recognized clinical disease correlated with elevated or significantly elevated levels of BF9 markers in conjunction to as well as prior to recurrence and disease progression. Similarly, patients 12 and 14 (FIG. 10) who have undergone neo-adjuvant therapy prior to surgery, show a steady increase of BF9 marker polypeptides in conjunction with disease progression (e.g. patient 14 immunotherapy/radiation therapy at day 303) and prior to metastasis

In patient 13 a significant increase in BF9 biomarker levels about one year after diagnosis may be indicative of ineffective treatment and predictive of upcoming metastasis which is clinically detected 868 days later (2.4 years; FIG. 10). Bone metastasis is detected at day 1257 and radiation treatment initiated, then disease progresses to the lung (day 1349). However no clinical information is available on the treatment incurred by this patient after day 1257 to explain the return to normal levels at day 1975.

Patients 3, 6, and 9 who were treated with AC-Taxotere adjuvant therapy, present with high test levels at baseline (>36 μgE/ml cutoff; range 142.5-282.1 μgE/ml; 43-190 days post-surgery; FIG. 10). High level baselines may either represent marker spikes relative to certain therapeutic treatments, or signal the early development of metastases. For example, in patient 6, where closely monitored serial samples are available, we notice a sharp decrease in test levels from 210.7 μgE/ml at day 43 to 40.61 μgE/ml at day 66, strongly suggesting that, at least in this case, high baseline is due to a short term sporadic spike. However no additional clinical information and samples are available from patients 3 and 9 to confirm this hypothesis. Future validation studies using multiple serial samples collected over a short period of time, such as every 3 months, will be advantageous. In patient 6, after test levels appear stabilized at day 66, both lung metastasis at days 543 (85.53 μgE/ml) as well as the subsequent time point just prior to death at day 550 (62.99 μgE/ml) correlate with increased levels of BF9 markers with respect to day 66. In patient 3, an initial high baseline level (142.5 μgE/ml at day 190; FIG. 10) is followed by a marker decrease (131.2 μgE/ml at day 308) possibly consistent with the stabilization trend after initiation of treatment hypothesized above. The subsequent sample drawn at day 512 shows a marker increase (242.4 μgE/ml) consistent with the patient incurring radiotherapy at 1.4 year follow-up. At 5.5 year follow-up the patient is found with loco-regional as well as bone and lung metastasis, a clinical status that also correlates with elevated test levels (158 μgE/ml at day 2003). The patient is then treated with combined chemotherapy and monoclonal antibody therapy (Bevacizumab; day 2157) resulting in test levels returning to normal at least at this time point. However, over two years later (2710 days; 7.4 years) disease progression and bone metastasis are clinically diagnosed. Interestingly at 2610 days (7.25 years) disease progression could have been predicted by BF9 monitoring, as test level is found culminating at 430.9 μgE/ml 100 days prior to clinical diagnosis. This once again suggests that BF9 serial monitoring over the course of follow-up would provide a lead time over detection of symptomatic recurrence.

Patient 9 displays high test levels at day 54 (282.3 μgE/ml) but they stablilize over the course of treatment around 96.23 μgE/ml at day 139. Over two years later bone metastasis is detected with BF9 test levels measured at 91.84 μgE/ml (day 775), suggesting that micrometastases had developed over that period of time. No samples are available between day 139 and day 775 to know whether the test was elevated in that span of time. The patient is then treated with first line therapy, which is effective as the patient is found alive with disease over four years later, a status that correlated with normal test levels.

Patient 10 displays high test levels from day 72, when the patient is treated with adjuvant therapy and declared NED, until day 141 (221-283 μgE/ml). However test levels eventually decrease possibly indicating stabilization (100 μgE/ml; day 211). Then BF9 marker elevation (218 μgE/ml; day 232) is observed 621 days prior to the patient undergoing surgery for a second breast cancer at day 853. This case suggests once again that continuous BF9 monitoring would have informed the clinician about the ineffectiveness of treatment prior to the development of a second breast cancer thus prompting a change of treatment regimen and type.

Overall, elevated BF9 test levels were found in the majority of serum samples associated with clinical disease (77%). In contrast, in the 11 samples with annotated CA15-3 measurements, elevated CA15-3 (standard 30 U/ml cutoff) correlated in 45% of the cases with disease, leaving 54% of recurrence, metastasis and breast cancer cases undetected. This result confirms that BF9 assay outperforms CA15-3 in the detection of local and distant recurrences, as observed in the cross-sectional study above. Furthermore, the results of the blind pilot follow-up study show that most recurrence events were preceded by BF9 elevation. Indeed a test level increase with respect to the baseline level or to the preceding time point, in absence of treatment, correlated with the subsequent finding of clinical and symptomatic recurrence or the first clinical evidence of disease progression. In addition in the few cases where a number of samples were available prior to clinical diagnosis of recurrence, BF9 assay detected disease relapse with a lead time of 100 (patient 3), 356 (patient 8) and up to 621 days (patient 10). In conclusion, these data strongly support the notion that BF9 assay may be used to monitor and predict changes in patient disease status, providing proof-of-concept for BF9 as an assay for monitoring breast cancer recurrence.

Multiple studies have assessed the use of conventional serum tumor markers for monitoring disease recurrence. SE of 0-48.3% in local recurrence and 49.4-80.8% in metastatic breast cancer with distant recurrence (Pedersen, 2013; Guadagni, 2001) have been reported. Overall, CA15-3 performance is most sensitive in detecting metastatic breast cancer, while poor in detecting loco-regional recurrence. This is why available markers are solely recommended for use in monitoring distant recurrence (Harris, 2007; Duffy, 2006). Monitoring loco-regional and contro-lateral recurrence would be most advantageous since it has been shown that they are associated to a more favorable prognosis and higher survival (Schneble, 2014). Earlier detection of recurrence, at a time loco-regional treatment may be sufficient to effectively fight tumor spread, may indeed save patients the secondary effects of systemic therapies, and increase long-term survival, ultimately impacting on breast cancer mortality.

### Immunodetection of Secreted BF9 Markers in Urine

In a preferred embodiment of the present invention, we further detect the presence of BF9 marker polypeptides, fragments thereof, or BF9 binding motif in the urine of cancer patients versus normal controls, using BF9 monoclonal antibody and the MPAT, as described in detail in Example 13.

Briefly, urine samples were centrifuged to remove debris, and equal volumes were spotted, in a double-blind experiment, on the MPAT membrane either “as is” or upon dilution in Tris-Triton buffer. Because of its low protein abundance, we reasoned that attempting to concentrate urine proteins would actually lead to loss of potential biomarkers. As indicated in FIG. 11 legend, a variety of cancer patients was included in the experiment in a 47 sample set (Table 3). Considering the likely possibility of prostate cells shedding into urine, we included samples from prostate cancer patients, representing stages II and III (n=13). Samples from early stage colon cancer patients (n=10; stage I and II), from patients with benign colon disease (n=4) and inflammatory conditions of the colon (n=5) were included as well as some pancreatic cancer cases (n+5) and normal controls (n=10).

As illustrated in FIG. 11, while only background reactivity is observed in normal individuals, BF9 monoclonal antibody clearly reacts with urine proteins in all cancer groups: in colon, pancreatic and prostate cancer. Some reactivity is observed in the colon inflammation cases, while no reactivity at all is observed in the colon benign cases. Specifically, in this experiment and clinical sample set, BF9 antibody detects biomarkers or a fragments thereof carrying the BF9 binding motif in the urine specimens of 50-90% of colon cancer cases, depending on the sample conditions used (FIG. 11, compare reactivity of samples “as is” versus samples diluted 1:2 or 1:10 in Tris-Triton buffer). Lower reactivity is observed in the urine specimens of colon inflammation cases. It should be noted that all colon cases in this experiment are stage I and II. This result emphasizes the ability of BF9 to detect early stage cancer.

With respect to the other cancers, BF9 assay detects 92% of prostate cancer patients (mostly in early stage disease) when urine samples are diluted 1:2 in Tris-Triton buffer (FIG. 11, row D), and of 4 out of 5 pancreatic cancer patients (nearly all early stage) in the same sample conditions (FIG. 11, lanes A and B; 1:2). In conclusion, in this experiment and clinical sample set, BF9 reactivity is observed in the urine samples of of colon, pancreatic and prostate cancer patients, suggesting that polypeptides or fragments thereof bearing BF9 binding motif are secreted in urine.

Pancreatic cancer, while only representing 6% of estimated new cancer cases in 2012, it is also responsible for 11% of cancer deaths (Siegel R et al., Cancer statistics, 2021, CA Cancer J Clin 62:10-29, 2012). In its early stage, pancreatic cancer is a relatively symptomless disease; hence patients generally present at advanced stage, and only 10-15% of them are surgical candidates, with small resectable cancers (ACS, 2012). For all stages combined, the 1-year relative survival rate is 24%, and the 5-year rate is about 4% (ACS, 2012). Pancreatic cancer has the shortest life expectancy of all malignancies when discovered, with a median survival rate of ˜18 months (ACS, 2012). Furthermore, serum biomarker CA-19, which is elevated in the advanced stage of several malignancies, particularly gastrointestinal cancers, is more clinically useful as prognostic and treatment response marker rather than early detection marker (Goggins, 2005; Duffy, 2010). Hence there is a great need for pancreatic cancer biomarkers, whether based on tissue or biological fluid, with particularly emphasis in the early detection of this malignancy.

Limitations of PSA as early detection marker for prostate cancer have been mentioned above, suggesting the need of additional biomarkers as adjunct to current screening methods, with particular emphasis in urine-based in vitro diagnostics.

### EXAMPLES

The following abbreviations are used throughout. hr: hour; min: minutes; sec: seconds; rpm: rotation per minute; RT: room temperature; ON: overnight; mAb: monoclonal antibody.

### Example 1: Epitope Analysis by Phage Display

As part of a characterization of the BF9 mAb, a phage display approach was employed using the New England Biolabs PhD-12 Phage Display Library Kit according to the manufacturer's instruction manual. A brief description of the protocol follows.

The phage library has a titer of 10¹³ pfu/ml. Ten microliters of the library are incubated in TBST (50 mM Tris-HCl PH 7.5, 150 mM NaCl, 0.1% Tween 20) with the mAb BF9 for 1 hr at RT. The mAb is immobilized on a plastic surface, such as an ELISA 96 well plate, via a rabbit anti-mouse IgG (see below). This represents a ˜10¹¹ pfu input, i.e. a ˜ 100 fold representation of a library with a complexity of 10⁹ individual clones, each harboring five copies of a 12-mer peptide embedded in the phage capsid. After incubation and washing of unbound phages in TBST, bound phages are eluted in 0.2 M glycine-HCl pH 2.2, 1 mg/ml BSA, then neutralized in 1 M Tris-HCl PH 9.1 and finally amplified in the appropriate bacterial strain (ER2731) by growing for 4-5 hrs at 37° C. with vigorous shaking. Phage is collected upon removal of bacterial cells by centrifugation, and precipitated by 20% PEG in 2.5 M NaCl at 4° C. ON. Phage is titered in order to carry out a second and third panning with an input titer equivalent to that of the first round. Note that stringency increases from the first to the third panning, by increasing Tween 20 concentration (0.1% to 0.5%) and decreasing incubation time (1 hr to 30 min).

To decrease non-specific binding of the phage library, the three panning steps are first preceded by a pre-panning (prior to incubation of phage library with the relevant mAb), whereby the phage library is incubated with non-specific or pre-immune mouse IgG immobilized to 96 well plates via a rabbit anti-mouse IgG. This pre-panning incubation step eliminates all phages in the library that would non-specifically bind to plastic, rabbit anti-mouse IgG and mouse IgG. Rabbit anti-mouse IgG plate coating is used to concentrate the relevant mAb (or the mouse lgG in the pre-panning step), which is in the form of hybridoma culture supernatant rather than in the form of purified mAb; such coating is also used to enhance mAb binding to the plate. Pre-panning is carried out ON at 4° C. or 1 hr at 37° C.

Upon the three cycles of panning and amplification, an enriched population of phage is collected; this enriched population harbors, within the capsid amino acid sequence, those 12-mer peptides that are specifically bound by mAb BF9, and thus represent, at least in part, the epitope of BF9 marker polypeptides. In fact they are preferably called mimotopes to indicate that they comprise sequences displaying homology to the actual epitope yet not the exact actual sequence.

After the third round of panning, resulting phages are titered (yet not amplified), and about 20 individual phage plaques are picked, amplified in a small volume of bacterial culture, and phage DNA is prepared upon a simple phenol chloroform extraction followed by ethanol precipitation. Phage DNAs are sequenced and the 12-mer peptide amino acid sequence is determined for each clone.

Non-redundant individual phages (i.e. with different 12-mer sequences) are amplified in medium sized cultures and titered in order to be further assayed by direct ELISA in the presence of mAb BF9. This ELISA assay will identify the clone(s) harboring the best 12-mer binders, as illustrated in FIG. 1. Comparison of the best binder sequences in turn provides insight into mAb BF9 epitope.

Briefly, ELISA plates are first coated with the mAb of the present invention (10-100 μg/ml, diluted when necessary in 0.1 M NaHCO₃ pH 8.6) by ON incubation at 4° C., or 1 hr at 37° C. Each step is followed by 10 TBST washes (0.5% Tween 20). Plate wells are then blocked with blocking solution (see above) for 1-2 hr at 4° C. Serial four-fold dilutions of phage to be tested (from 10¹² to 2×10⁵ virions per well) are prepared in a separate pre-blocked 96 well plate in TBST (0.5% Tween 20), and added to mAb BF9 coated wells, and to the uncoated (but blocked) wells used as negative controls, for 1-2 hr incubation at RT with agitation. Phage-mAb complexes are detected by colorimetric reaction at 450 nm involving a 2-step process: i) 1 hr incubation at RT with horse radish peroxidase (HRP) conjugated anti-M13 antibody according to manufacturer's instructions (GE Healthcare), followed by ii) addition of TMB (HRP substrate) until a blue color develops. Signal intensities are compared to the “no mAb” control, and the reaction stopped by H₂SO₄.

### Example 2: Determination of BF9 Marker Polypeptide Identity

The sequence of the 12-mer peptides, selected by the phage display approach, that have shown to bind with highest affinity to BF9 monoclonal antibody (e.g. best binders, as described in Example 1 and listed in FIG. 1 and herein) are used to query NCBI protein databases (Non-redundant protein sequences (nr); Reference proteins (refseq_proteins); UniprotKB/swissprot (swissprot)) by protein BLAST (blast.ncbi.nlm.nih.gov). The 12-mer peptide sequences are referred to as mimotopes to indicate that they comprise amino acids displaying homology to the actual BF9 epitope. Therefore the BLAST search yields proteins featuring an amino acid sequence with high homology to the BF9 mimotopes, thus providing in turn the identity of the BF9 polypeptides recognized by BF9 monoclonal antibody.

In addition to phage display used to characterize monoclonal antibody BF9, human expression library immunoscreening, and proteomics (immunocapture mass spectrometry were used to identify the BF9 biomarker polypeptides recognized by the BF9 antibody. In one of the embodiments, we screened high-density protein macroarrays containing 27,648 individual E. coli expressed protein clones from a human fetal brain cDNA expression library with the BF9 monoclonal antibody (Bussow et al., 1998). Briefly, macroarrays were washed twice in TBSTT (TBS, 0.05% Tween 20, 0.5% Triton X-100) to lyse bacteria and four times in TBS. After blocking with 3% non-fat dray milk in TBST (TBS, 0.05% Tween 20) for 2 hr at RT, macroarrays were incubated overnight with BF9 hybridoma supernatant (diluted 1:5 or undiluted) either 2 hr at RT or ON at 4° C. Macroarrays were washed three times with TBST and exposed for 1 hr t RT to anti-mouse IgG infrared 800 (Li-Cor) as per manufacturer recommendation. After four washes in TBST and two in TBS, signals were detected by scanning with Odyssey imager at the 700/800 nm channels. Macroarrays were analyzed using scoring templates from manufacturer to locate coordinates of duplicate spots, and then referring a particular clone to the RZPD database (https://www.ebi.ac.uk/arrayexpress/files/A-GEOD-15009/A-GEOD-15009.adf.txt).

In another embodiment we used BF9 mAb to pull down the corresponding antigens from MCF7 protein extracts and SFCM by immunoprecipitation. Captured proteins were separated by SDS-PAGE and Coomassie blue stained. Bands were cut off from the gel and subjected to in-gel trypsin digestion followed by MS analysis. Following Scaffold protein identification software analysis, proteins were selected from MS data hits, based on MS signal intensity, indicating protein abundance, and percentage of peptide coverage, resulting in candidate biomarkers whose sequences shared homologies with mimotopes evidenced by phage display.

### Example 3: Matrix Protein Array Screening Technology

The matrix protein array technology (MPAT) is a multiplex protein array immunoassay developed by the Applicant for the simultaneous analysis of multiple biological samples, under the same conditions. The MPAT has been used for the immunodetection of protein marker/mAb of the present invention in a variety of protein samples, as detailed in the examples below.

The solid support of the matrix protein array may be composed of a different number of chambers or compartments of different sizes, depending on the scope of the investigation. In its simplest format, the MPAT is composed of 96 chambers. Other formats can be used, depending on the number of antibodies to assay, and the number of samples to screen. Biological samples are spotted or printed (see below) in a matrix arrangement within each compartment on a nitrocellulose membrane. The same matrix of clinical samples, including normal and diseased, or the same matrix of protein extracts from different cancer cell lines is printed in each chamber. Each individual compartment is then overlayed with a distinct antibody (polyclonal, monoclonal, Fab fragment, monospecific, single chain, affibodies, or any other recombinant version of conventional or combinatorial antibodies), and processed for the detection of antigen-antibody complexes.

Protein sample analysis. For the purpose of the invention, protein samples analyzed by MPAT may derive from fresh and frozen tissues, whether normal or disease, including from patients with cancer, benign or inflammatory conditions, and normal controls. Protein samples may derive from cell cultures, cancer cell lines, and cancer cell supernatants, and even from microdissected cell types or from a given subcellular compartment. Protein samples may also derive from patient sera or any other patient biological fluid, and prepared as described in the Examples below.

Printing of total protein extracts. Individual protein sample extracts can either be deposited and spotted manually or printed with a robotic system (Genomic Solutions Flexys, PBA Robotics, UK). Routinely equal protein amounts (250 nl of a 1 mg/ml stock solution of cancer cell line protein extract) of each sample are printed in a matrix format on the MPAT membrane, in duplicate or triplicate whenever deemed appropriate. The membrane is then incubated for 30 min in 2% H₂O₂ (hydrogen peroxide) solution to inhibit endogenous peroxidase present in the clinical samples, rinsed twice in Tris-saline buffer (TNE: 10 Tris-HCl PH 7.5, 50 mM NaCl, 2.5 mM EDTA) and then blocked for 30 min with a solution of 1% non-fat dry milk in Tris-saline buffer containing 0.1% (w/V) Tween 20 (TNET).

Antibodies. Subsequently, each chamber or each sample matrix is overlaid with a given primary antibody. Routinely antibodies are diluted appropriately in blocking solution, followed by 1 hr incubation at RT with constant shaking. Blocking solution is TNET containing 1% non-fat dry milk or equivalent blocking solutions.

Detection of antigen-antibody complexes. The membrane is washed 5 times for 5 min each in TNET, then incubated for 1 hr with secondary antibodies conjugated with horseradish peroxidase (Roche) diluted 1:10,000 in blocking solution. The membrane is then further washed 5 times as described previously. The antigen-antibody-anti-antibody complex reactivity is measured by chemiluminescence, using the SuperSignal West Dura Extended Duration Substrate (Pierce). The image is captured using a CCD-camera (charge-coupled device; UVP model Biochemi, CCD camera grade 0, with dark room designed for chemiluminescence, fluorescence and visible).

Alternatively, instead of a chemiluminescent-based detection and a CCD-camera based image acquisition system, a fluorescent-based system can be used, incorporating for example the use of the Li-cor Odyssey infrared imaging acquisition system. The MPAT protocol is then modified accordingly. Peroxidase inhibition is not necessary. The membrane is rinsed twice in Tris-saline buffer, and then blocked for 30 min in Odyssey blocking solution (Li-Cor). Primary antibody is appropriately diluted in Odyssey blocking solution, followed by 1 hr incubation at RT. The membrane is washed 5 times for 5 min each in TNET, then incubated for 1 hr with secondary antibodies labeled with a fluoresecent dye (lgG-IRDye 800CW) diluted 1:10,000 in Odyssey blocking solution. The membrane is then further washed 5 times as described previously. The antigen-antibody-anti-antibody complex is measured by direct infrared fluorescence detection. The intensity of each complex is captured as an image by scanning the membrane with Odyssey infrared imaging system in the 800 nm channel at 84 μm resolution. Protocols based on different labeling and detection systems, such as alkaline-phosphatase, biotin-streptavidine, and fluorophores as described can also be successfully performed within the scope of the present invention.

The following internal controls can be routinely provided: i) the same matrix of samples is overlaid with buffer rather than with primary antibody, followed by the secondary antibody, thus revealing the background of the secondary antibody (no antibody control); and ii) the same matrix of samples is overlaid with pre-immune serum, or non-secreting hybridoma or dilution buffer, followed by secondary antibody, thus revealing the nonspecific binding of mouse immunoglobulins.

### Example 4: Immunodetection of BF9 Marker Polypeptides by Matrix Protein Array in Patient Tissues

Clinical samples. Frozen human tissue biopsies with annotated pathology report were acquired from the Cooperative Human Tissue Network (CHTN). All specimens are tissue samples collected prior to any treatment. Specimens are provided with corresponding pathology report and well-annotated clinical information (disease condition, cancer histological type, clinical history, stage, age, gender, race; Jewell, 2002). Clinical samples are stored in −70° C. freezers, and immediately prior to assay, samples are aliquoted in ice to avoid multiple freeze-thaw cycles, and the original tube is maintained at −70° C. The collection amounts to ˜1,500 cancer tissues covering all major malignancies, comprising colorectal cancer, lung, pancreatic and prostate cancers, melanoma, renal carcinoma, and gynecological cancers, including breast, ovarian, uterine, and cervical cancers.

Protein extraction from frozen tissues. Fresh or frozen tissue of human origin for the purpose of this invention, is cut off in small pieces, grounded, homogenized in a 50 mM Tris-HCl PH 7.5, 2 mM EDTA, 100 mM NaCl, 1% NP40, and 1 mM vanadate solution containing the following protease inhibitors: PMSF, aprotinin, leupeptin at 1, 2 and 4 mM respectively. The homogenate is kept on ice for 20 min and centrifuged at 14,000 rpm for 15 min. Supernatant is transferred to a new container and the tissue pellet is resuspended, and again kept on ice for 20 min and centrifuged as indicated above. Supernatant is removed and added to the first one. Protein concentration is determined according to standard conditions as known to those skilled in the art. Protein solution is stored in a −80° C. freezer until further usage.

MPAT. Protein extracts from frozen tissues are spotted on the MPAT and processed as described in Example 3 using mAb BF9.

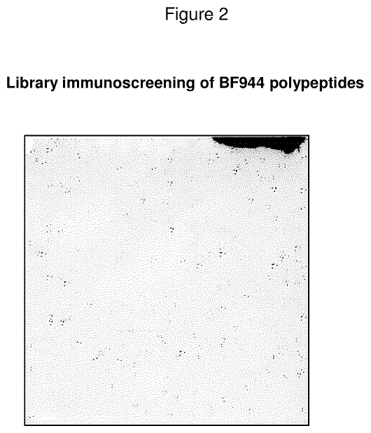

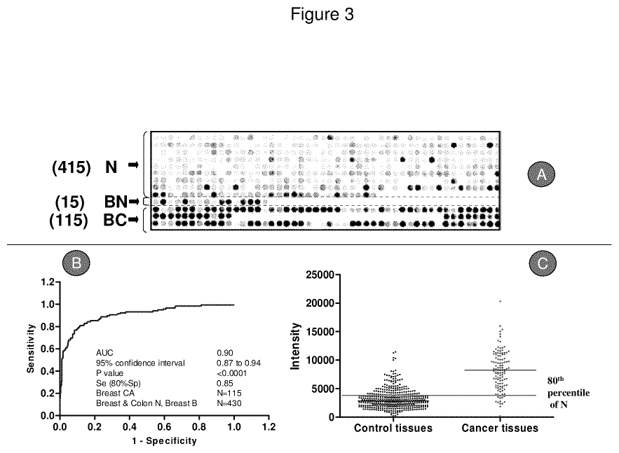

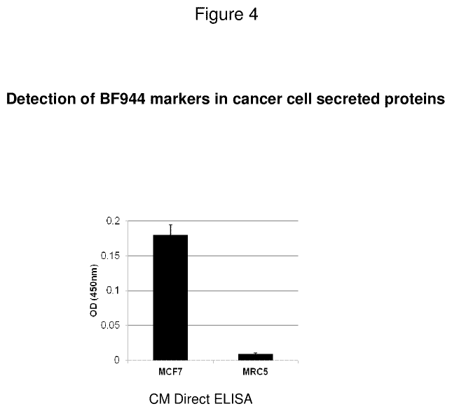

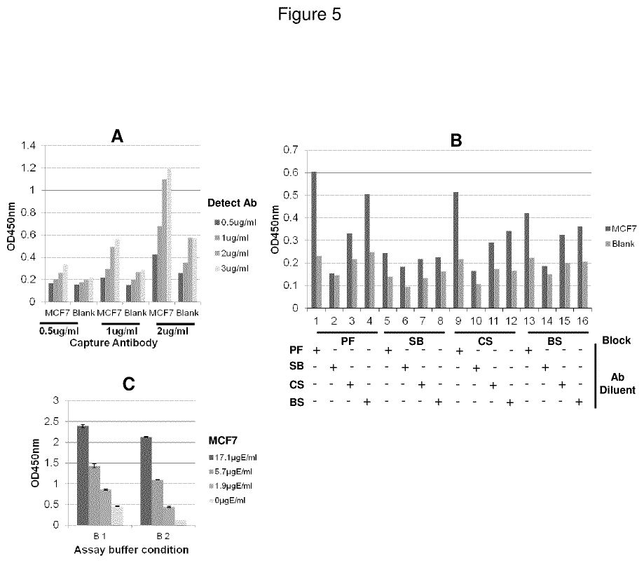

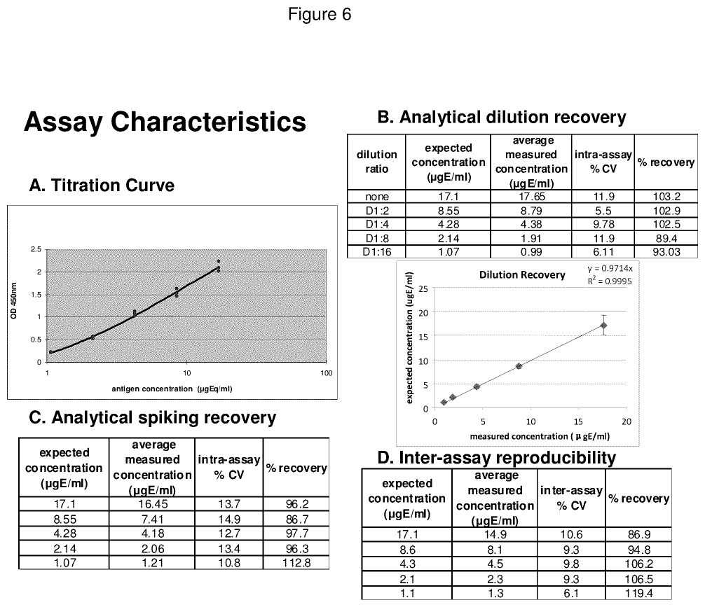

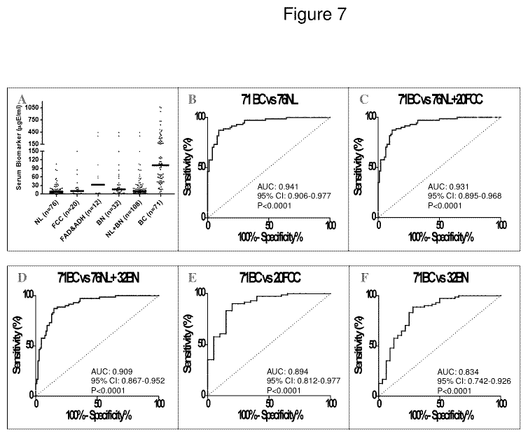

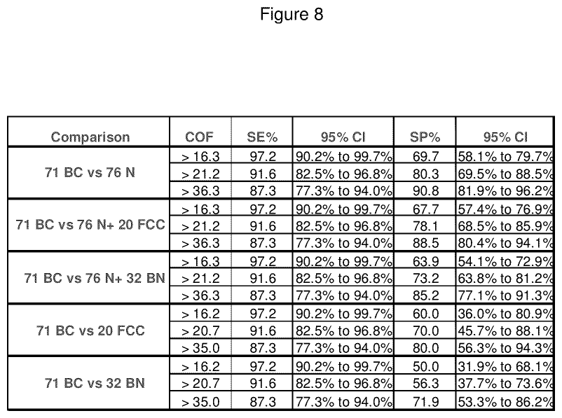

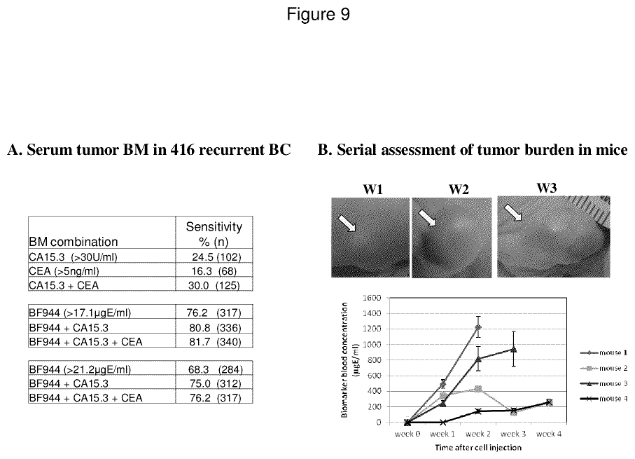

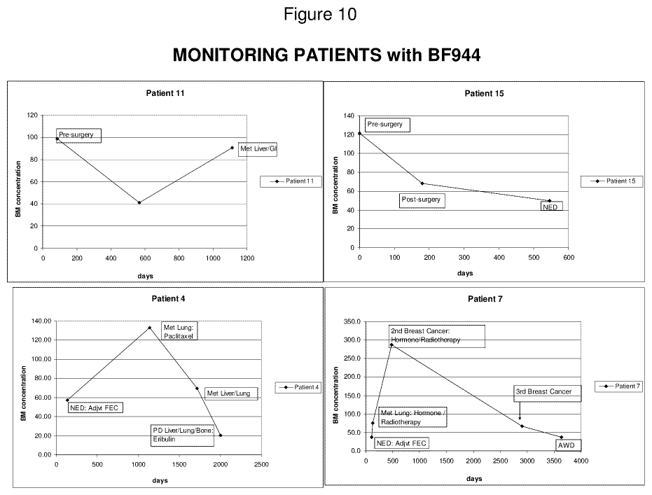

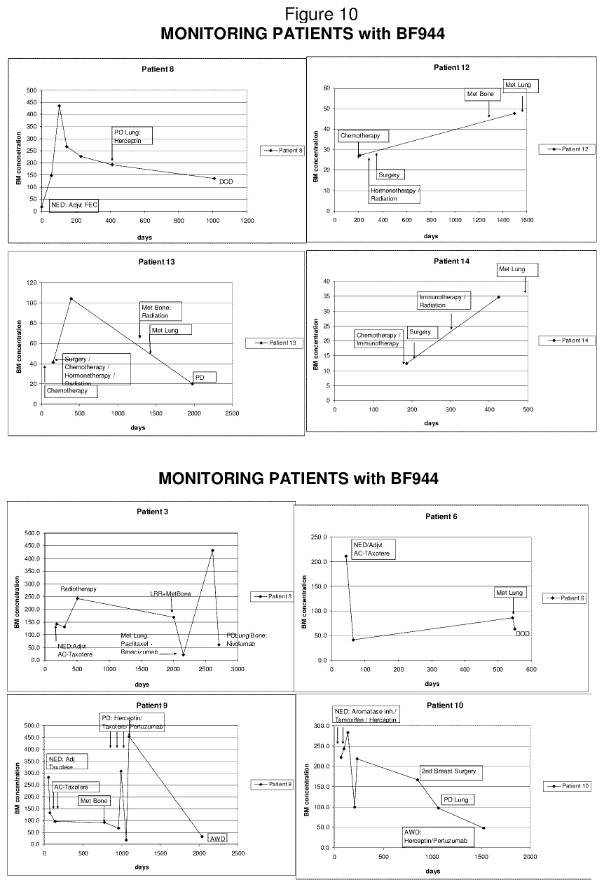

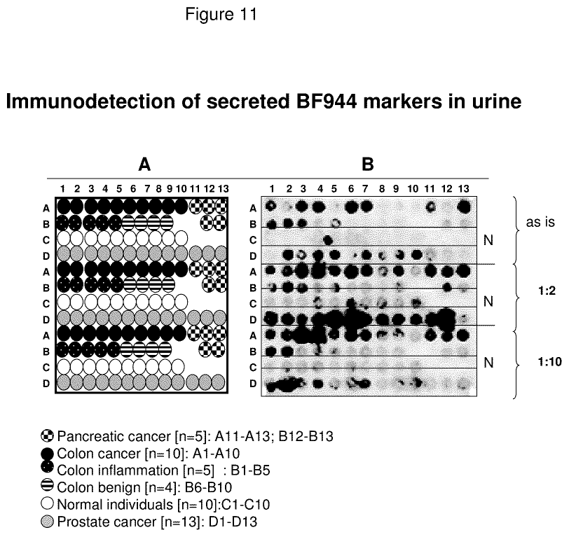

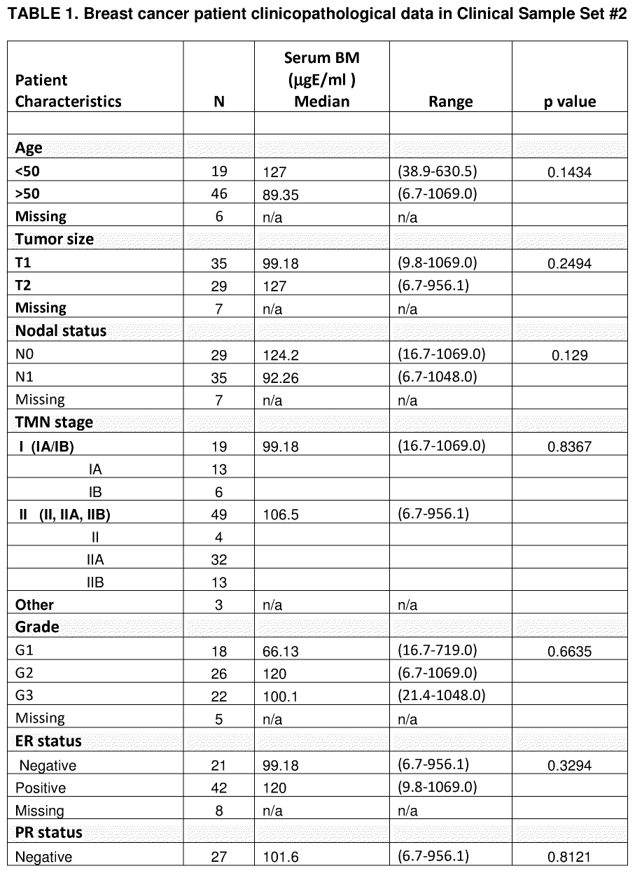

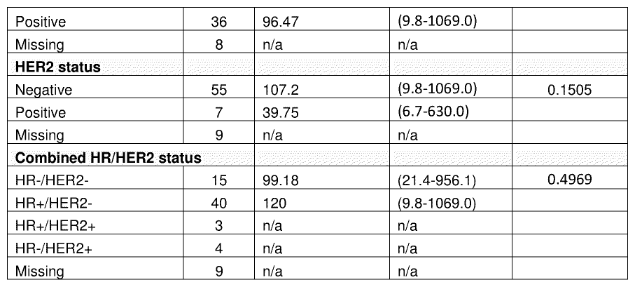

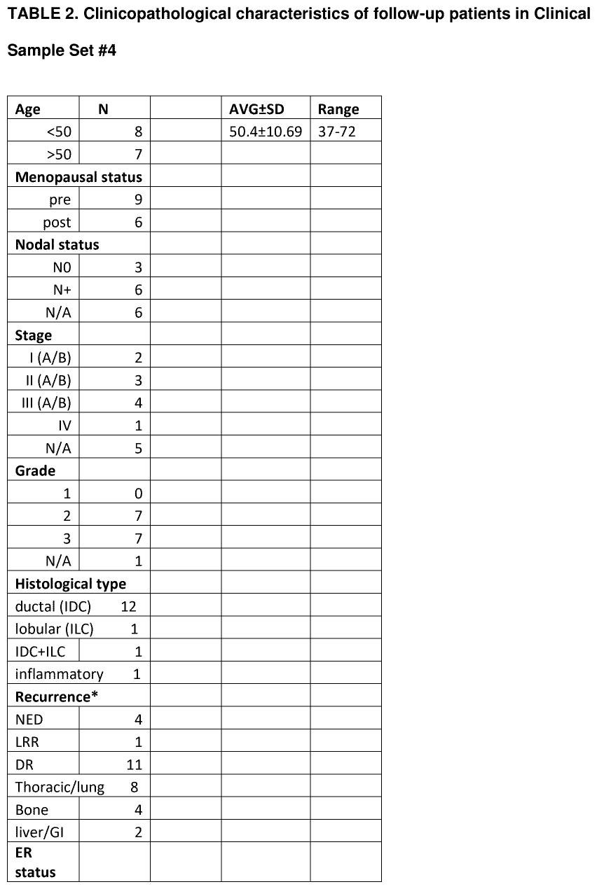

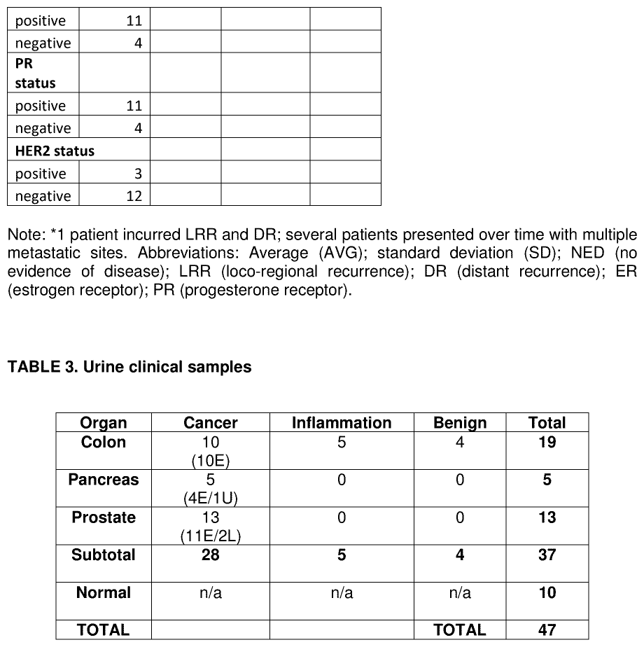

Data Analysis. MPAT spots are quantified by the ScanAlyze2 program (Eisen, 2002) which enables robust and high-throughput spot finding on the scanned image produced by the Li-Cor Odyssey infrared imaging system. Quantification of the MPAT spots produced by the reaction between BF9 mAb and the antigens it recognizes within patient samples is illustrated in one embodiment of the present invention (FIG. 2). ScanAlyze2 quantifies spots by determining the average intensity of all the pixels in the spot. Such intensity quantification provides, for each spot, a value indicating the relative expression level of the corresponding antigens in the samples. Scatter plots are generated with spot intensity values obtained in each group (breast cancer, breast benign and normal controls) using GraphPadPrism statistical package software. Then the means of the intensity of the spots in each group of interest are compared using one way ANOVA analysis of variance, with Tukey multiple comparison test. This provides all statistically significant differences in intensity means in all possible paired comparisons (p value<0.05). Area under the curve (AUC), 95% confidence intervals (CI), sensitivity and specificity of BF9 mAb are determined from receiver operating characteristic (ROC) curves. The discriminatory power of BF9 mAb in a given comparison as determined by AUC values, is not considered statistically significant when the 95% CI is large and contains 0.5 or chance line.

### Example 5: Immunodetection of BF9 Marker Polypeptides in Cancer Cell Lines

Cancer cell lines such as MDA-MB 231, and MCF7 (breast cancer); COLO 320DM, DLD-1, and WiDr (colon cancer); NCI-H157, H460 and SKLU-1 (lung cancer); PC-3, DU 145 and LNCaP (prostate cancer).

Preparation of protein extracts from cancer cell lines. Cancer cell lines (about 107) are grown in culture as recommended by ATCC provider, with 10% fetal calf serum, 100 μg/ml streptomycin and penicillin until 80% confluency, harvested, washed twice with PBS, resuspended in phosphate buffer (pH 8.0) and disrupted in the following buffer: 50 mM Tris-HCl PH 7.5, 2 mM EDTA, 100 mM NaCl, 1% NP40, and 1 mM vanadate solution containing the following protease inhibitors: PMSF, aprotinin, leupeptin at 1, 2 and 4 mM respectively. The cell lysate is centrifuged for 5 min at 14,000 rpm. Protein concentration of cancer cell extracts is determined using the BCA (bicinchoninic acid) Protein Assay Reagent Kit (Pierce, Rockford, IL) using a 1:200 dilution of extract, and a BSA standard. A microplate reader (vmax, Molecular Device) is used to read the absorbance at 570 nm. Stock solutions of protein extracts at 1 mg/ml are used.

### Example 6: Staining of Cancer Tissues by Immunohistochemistry

To demonstrate the specificity of the markers and monoclonal antibody of the present invention, and their use in histology-based diagnostic applications, tissue slides or tissue arrays displaying tissues from cancer and benign patient, and normal controls (matched, i.e. from the same patient, or unmatched) are used as follows.

5-um formalin-fixed paraffin-embedded human tissue section slides or tissue microarrays are deparaffinized by baking slides in oven at 60° C. for 30 min followed by immersion in three xylene baths for 5 min each. Slides are rehydrated by immersion in two 100% ethanol baths for 5 min each, then in 95% ethanol, 70% ethanol baths for 3 min each, and finally soaked in water.

Endogenous peroxidase is blocked by treating slides with 3% hydrogen peroxide solution in PBS for 10 min at RT, then washing them twice in PBS for 3 min each. Antigen retrieval is obtained by heating slides in a pressure cooker at full pressure for 5 min in 10 mM Tris, 1 mM EDTA pH 9, or in Tris-sodium citrate 10 mM, 0.05% Tween 20 pH 6. Slides are then cooled to RT in the same buffer for 10-20 min, rinsed in tap water for 3 min, and finally immersed in Tris buffer.

To block endogenous biotin, which may interfere in some tissues with the detection systems, slides are incubated for 15 min at RT in a streptavidin solution in PBS (100 μg/ml), rinsed with Tris buffer, followed by incubation with biotin solution (500 μg/ml) in PBE (PBS with 1% BSA, 1 mM EDTA, 1.5 mM NaN₃ pH 7.4) for 30-60 min at RT, and washed in PBS. Non-specific binding is further blocked by treating slides for 15min at RT in 3% horse serum diluted in PBE.

Slides are incubated with mAb BF9 (either undiluted cell culture supernatant, or appropriately diluted 1:2-1:20 in PBE buffer) for 30 min at 37° C., or 1 hr at RT or overnight at 4° C. in a humidity chamber, then rinsed 3 times for 5 min each in Tris buffer. Slides are covered with a 1:1000 dilution of biotinylated secondary antibody in PBE buffer, and incubated for 30 min at 37° C. or 1 hr at RT, then washed 3 times for 5 min each in Tris buffer. Slides are then covered with 1:1000 dilution of peroxidase-conjugated streptavidin diluted in PBE (without azide), and incubated for 30 min at 37° C. or 1 hr at RT, then washed 3 times for 5 min each in Tris buffer.

Finally, a few drops of AEC substrate solution (1 ml of 4 mg/ml AEC stock solution in DMF, plus 15 ml of 0.1 M Na acetate pH 5, and 15 μl of 30% hydrogen peroxide) are used to cover the slides. The reaction is allowed to pursue for 10-40 min, then visualized under the microscope, and stopped with tap water whenever appropriate. Slides are rinsed in water, and counterstained with a few drops of weak Mayer's hematoxylin solution for 1-2 min. Slides are then immersed in 0.1% sodium bicarbonate solution until nuclei turn blue. Slides are covered with aqueous mount media, placed in an oven at 70° C. and then let dry for 10-20 min or overnight at RT.

### Example 7: Western Blot Analysis

Total protein extracts (equivalent to 10 microgram per lane) from a given tissue or cancer cell extract are loaded, separated on polyacrylamide-SDS and transferred onto nitrocellulose membrane according to standard procedures. Different percentage of polyacrylamide may be used as known by those skilled in the art, depending on the expected molecular weight of the marker, and nitrocellulose can be replaced by PVDF, nylon membrane or other support. After transfer, the membrane is saturated for 1 hr in TNE/Tween blocking buffer (10 mM Tris-HCl pH 7.5, 2.5 mM EDTA, 50 mM NaCl, 0.1% Tween 20) containing 2.5% dried non-fat milk. The membrane is used as is or cut into strips of different size as necessary. Each membrane section or strip is first blocked with BSA or other commonly used blocking agent, then incubated for 1 hr with an antibody at appropriate dilution in the blocking buffer.

The blot is washed 5 times in the same buffer described above and incubated for 1 hr with a goat anti-mouse secondary antibody conjugated with IRDye 800CW fluorescent dye (Li-cor) according to manufacturer's instructions. Then membrane sections or strips are washed 5 times for 10 min each in TNE/Tween without milk. Antigen-antibody complexes are visualized by scanning the membrane sections or strips using the Odyssey infrared Imaging System (Li-cor) according to manufacturer's instructions. Other detection systems, known to the skilled in the art, can be used as well.

### Example 8: Detection of Secreted BF9 Markers From Cancer Cell Lines

Cancer cell lines. To test for the presence of secreted BF9 markers in cancer cell lines, cell culture supernatants were assayed with mAb BF9.

Preparation of cell culture supernatants. Tissue culture supernatants (TCS) are centrifuged to remove cell debris and supernatants are precipitated by slow addition of 1-1.5 volumes of ice-cold acetone. Precipitation is carried out on ice or at −20° C. for 1 hr. After 15 min centrifugation at 4° C. using pre-cooled rotors, tubes are inverted to completely remove supernatants. Pellets are quickly recentrifuged to fully eliminate the last drops of supernatant. Finally, pellets are allowed to dry for 5-10 min under the hood and resuspended in 2.5 ml of Tris 50 mM pH 7. Samples are homogenized with sonicator, whenever needed, and protein concentration is measured via a BCA assay (see above). Samples are diluted to 1 mg/ml working solutions.

To prepare serum free culture medium (SFCM) to facilitate analysis of potentially secreted proteins by immunodetection analysis of cancer cell lines by MPAT, cells are grown to 70% confluency, complete medium is removed and replaced with medium without fetal calf serum and grown for 25 hr at 37° C. SFCM is then precipitated as above.

### Example 9: BF9 ELISA Assay

A typical sandwich ELISA assay based on matched capture and detection mAb pair against the BF9 binding motif was used to detect BF9 polypeptides in patient serum. Nunc Maxisorp immunoplate wells were coated with 100 μl capture specific mAb in PBS, and incubated overnight at 4° C. The wells were then blocked with 350 μl of blocking solution for 1 hr at 37° C. and were washed three times with PBS. Antigen source (SFCM or serum samples), calibrators and controls were added to the wells and incubated for 2 hr at RT. The wells were washed four times with PBS containing 0.05% v/v Tween 20, and 100 μl of biotinylated detecting BF9 specific mAb diluted in blocking solution was added. After incubation for 1 hr at 37° C., the wells were washed as previously described, and 100 μl of streptavidin linked horseradish peroxidase (HRP; Jackson ImmunoResearch) was added according to manufacturer instructions. Wells were then washed with PBST, incubated with 3,3′,5,5′-tetramethylbenzidine substrate solution (TMB, Sigma) for color development, and the reaction stopped with 2M HCL. The absorbance was read at 450 nm with a microplate reader (vmax, Molecular Devices).

Antigen source. A SFCM (serum free cell culture medium) of the human breast cancer cell line MCF7 (ATCC HTB-22) was prepared as described (Kulasingam, 2007; Luka, 2011), and used as surrogate antigen (Ag) source. Cells were grown in SFCM to ensure that the conditioned media contained no other exogenous proteins. Multiple SFCM collections were pooled in a large batch, aliquoted in small fractions to avoid multiple freeze-thaw cycles, and stored at-80° C. A small aliquot was used to measure total protein concentration (expressed in ugEquivalent/ml) by a BCA protein assay kit (Pierce).

Antibodies. Matched capture and detection mAb pair against BF9 were protein A/G purified from mouse ascites, dialyzed against PBS, aliquoted in small volumes to minimize multiple freeze-thaw cycles and stored at-20° C. Detection mAb was also biotinylated using Sulfo-NHS-LC-biotin (Pierce) according to manufacturer's instruction. MAb concentration was determined by BCA protein assay. Biotinylated mAb was stored in small aliquots at-20° C. Studies herein were performed with the same lots of mAbs. Working concentrations of mAbs were determined by chessboard titration of both capture and detection mAb using constant concentration of antigen source. The optimal working concentration was defined as the one yielding the maximum signal to noise ratio with an acceptable non specific binding (OD<0.2) in assay diluent.

Calibrators, controls and standard curve. Zero (0) calibrator is a serum pool from normal donors (Sigma) with undetectable level of the antigen. A 10× standard calibrator stock was prepared by mixing a known quantity of BF9 antigen source with the zero calibrator. In addition, two positive controls were prepared by pooling serum samples from breast cancer patients, previously identified as having high or low amount of BF9 marker levels. Large volumes of 10× standard calibrator stock, zero calibrator and positive controls were aliquoted and stored at −80° C. On the day of the assay, unknown serum samples, positive controls, zero and standard calibrators were diluted 1:10 in assay diluent (to 10% final serum concentration). A standard curve is constructed by measuring the absorbance of a serially diluted 1× standard calibrator using the 10% zero calibrator as diluent. Each assay plate contains standard curve calibrators, positive controls and unknown samples. Measurements of calibrators, controls and unknowns were done in duplicate, and the average was taken as final reading. Unknown samples producing ODs outside the range of the standard curve were re-assayed at an appropriate dilution until they fit the range of the standard curve. BF9 level (μgE/ml) in patient serum was interpolated from the standard curve generated by 4 parameter logistic model (R2=0.992).

Assay quality control. To ensure reproducibility between different assay runs, standard curves were plotted as ODs normalized between maximal and minimal values obtained on the same microplate as a function of Log [Ag concentration]. Statistical comparison of multiple standard curves was done using nonlinear regression curve fit (GraphPad Prism7.0). Midpoint antigen concentration (C50) values were calculated from the sigmoid curves and did not show statistically significant difference (p>0.05; F-test). If the results of the assay did not fit the established acceptable range of controls values, they were considered invalid.

Analytical validation. To examine the analytical dilution recovery, a known amount of BF9 surrogate antigen is spiked into assay diluent supplemented with 10% of a normal human serum pool containing undetectable levels of said antigen (zero calibrator). The sample is then serially diluted in assay diluent, in quadruplicate samples, as indicated. In each of the 5 dilutions tested, the measured antigen concentration is derived from the standard calibration curve, and the average measured concentration of quadruplicate values (SD) is plotted against the expected concentration. Then the ratio between the expected antigen concentration and the average measured concentration is calculated thus yielding a % recovery. Each % recovery result is within the acceptable range of 80-120%. The percent coefficient variation (% CV) is defined as the ratio of the standard deviation (SD) over the mean x 100 (SD/mean*100). % CV of quadruplicate values are within acceptable limits (<15%). Dilution linearity is defined by a regression coefficient R2 value of ˜1. Regression analysis yields a slope of 0.971 and R2 =0.999, indicating linear dilution in the range of concentrations tested. To measure the analytical spiking recovery, a normal human serum pool diluted 1:10 in assay diluent containing undetectable levels of antigen, is spiked, in quadruplicate, with 5 amounts of BF9 surrogate antigen, ranging from 1.07 to 17.1 μgE/ml. Recovered antigen concentration is measured and interpolated using the BF9 calibration curve. Quadruplicate values of each measured concentration are averaged. % recovery and % CV are calculated. To determine the inter-assay reproducibility, the spiking recovery experiment using 5 different concentrations of antigen was repeated in triplicate and in 3 independent days to measure inter-assay variance. % recovery and % CV of 9 measurements are within acceptable limits.

### Example 10: Statistical Analysis of Data

Statistical analysis, ROC curves and scatter plots were performed with GraphPad Prism7.0 (GraphPad Software, Inc., La Jolla, CA). The median biomarker concentration values in each group were compared to determine whether differences were statistically significant (p<0.05). The relationship between clinicopathological parameters and serum biomarker levels was examined as well (Table 1). Statistical analysis of two-group and multi-group comparisons was carried out using the non-parametric Mann-Whitney U test and the Kruskal-Wallis test, respectively. Receiver operating characteristic (ROC) curves were calculated to evaluate the diagnostic performance of the BF9 assay in various comparisons, by determining sensitivity (SE), specificity (SP), area and curve (AUC), and confidence interval (CI). For statistical analysis of age in different groups (when distribution passed D′Agostino & Pearson normality test) we compared the age means+SD using one-way ANOVA followed by Dunn's multiple comparison test.

### Example 11: Xenograft Models

A suspension of 5×106 MCF7 breast cancer cells in PBS was injected s.c. in each of 4 male athymic NCR nu/nu mice 7 to 8 weeks of age. Standard protocols were used (Morton, 2007). Tail vein blood (200 μl) was drawn from each mouse prior to injection, and every week thereafter, and corresponding serum was assayed in duplicate to measure BF9 levels. Tumor growth was visually inspected. Animals were kept in a pathogen-free animal facility and maintained according to PHS guidelines on Animal Welfare Assurance.

### Example 12: Clinical Samples (Serum)

Clinical samples were carefully selected to address two major clinical applications: breast cancer (BC) early detection and monitoring. We used four independent serum sample sets, described below. Clinicopathological features of breast cancer patients from the Clinical Sample Set #1 and #2 are summarized in Table 1. For preliminary measurement of BF9 SE/SP we used serum samples from our in-house collection, which were all obtained through the Cooperative Human Tissue Network (CHTN; Jewell, 2002), a network of institutions and clinical centers throughout the US. All CHTN samples are collected under harmonized SOPs, and encompass a broad patient population, thus minimizing bias due to single sample source. CHTN samples were provided with highly annotated path report, including disease condition, cancer stage and histological type, age, gender, race, prior personal and family cancer clinical history, information regarding diseases other than cancers and possible medications. Samples were provided with no private identifiers. This sample set (referred to as Clinical Sample Set #1) was composed of 14 BC cases (11 ductal adenocarcinoma including 1 DCIS, 2 mixed ductal/lobular, 1 unknown; 12 stage I/II, 2 stage IV; Table 1), collected at surgery prior to any treatment, and 13 healthy women normal controls (NL).

For measurement of BF9 diagnostic performance, particularly in early stage, we used a second set of serum samples (referred to as Clinical Sample Set #2) that amounted to 71 BC (69 TNM stage I/II, 2 late stage; Table1), 32 benign including non-proliferative (20 fibrocystic changes, FCC) and proliferative conditions (11fibroadenoma, FAD; 1 atypical ductal hyperplasia, ADH), and 76 normal controls. In this second set, the BC group comprised all the samples from Clinical Sample Set #1 plus 57 additional early cancer cases acquired from the PATH collection. Breakdown of the PATH early stage ductal adenocarcinoma samples was: 7 T1NOM0, 21 T1N1M0, 15 T2N0M0 and 14 T2N1M0 cases, from women aged 40-70, pre or post menopausal, with known ER/PR/HER2 status and grade (Table 1). The age mean+SD in the BC group (n=71) was 60+12 years. The PATH Foundation (Patients' Tumour Bank of Hope) is a German based biobank, collecting tissue, serum samples, and clinical and socio-demographic data from breast cancer patients at seven different certified breast centers in Germany. PATH patient recruitment follows strict SOP for collection and processing, under HIPAA-equivalent procedures aimed at protecting the privacy of patients, as supervised by the Ethics Committee of Bonn University and the Bavarian Data Protection Agency (Waldmann, 2014). The additional breast benign disease (BBD) patients in Clinical Sample Set #2 came from the CHTN. The additional normal controls came from our in-house collection, or procured from the CHTN. The normal control group comprises apparently healthy women, with no history of any serious or chronic illness or cancer, and known menopausal status (n=76; age mean±SD: 44±14 years).

The third set of serum samples (referred to as Clinical Sample Set #3) addressing breast cancer monitoring was obtained from a third party clinical laboratory hospital specialized in BC, and comprises 416 BC patients who suffered a recurrence after primary cancer and with known CA15-3/CEA levels measured at hospital visit (AdviaCentaur assay, Siemens).

The fourth set of clinical samples (referred to as Clinical Sample Set #4) used for the pilot longitudinal study, was composed of 57 serum samples serially collected from 10 patients after primary breast cancer treatment, through recurrence and beyond (4-9 time points per patient; follow-up range from 0 until 9.9 years). In addition, 9 serum samples from 4 patients collected at different time points after primary BC treatment (follow-up for 6.2 years after diagnosis), and 3 serum samples (pre-surgery, post-surgery, and follow-up blood drawn) form one patient who remained disease free for 9 years completed this set. Clinicopathological features of follow-up samples are provided in Table 2 together with time of follow-up after diagnosis, clinical status (NED, PD, AWD, DOD), recurrence type (loco-regional or distant), distant recurrence site (bone, lung, liver) and patient treatments (adjuvant, hormonal, radiotherapy, Herceptin, surgery). Three of the patients who remained disease free (NED) for 2.5 to 7.9 year follow-up, served as controls.

All samples used in this study were received on dry ice and immediately stored at −80° C. until their use, then prior to assay, samples are aliquoted on ice to avoid multiple freeze-thaw cycles. Sample quality control involved verification of clinical information and path report, and removal of samples with evidence of excess lipids, or hemolysis. All patient samples and clinical information were collected under strict human subject guidelines, with IRB-approved protocol and patient informed consent in place at the original institutions. Samples were handled, and packaged for transport by providers according to US and EU regulations, and provided to Applicant in a de-identified manner.

### Example 13: Immunodetection of Secreted BF9 Markers in Urine

Clinical samples. The clinical sample set for the urine experiment comprised 47 samples as follows: 10 colon cancer patients (6 stage 1, 4 stage II), 5 with inflammatory conditions of the colon, 4 with benign conditions of the colon, 13 prostate cancer patients (11 stage II, and 2 stage III), 5 pancreatic cancer patients (4 stage II and 1 unknown), and 10 normal controls. Note that cancer stages I and II are defined as “early”, while stages III and IV are defined as “late”.

Preparation of urine samples. Unprecipitated urine samples are first centrifuged to remove debris whenever turbid, then 250 nanoliters of each urine sample are printed on the MPAT either “as is” or upon dilution 1:2 or 1:10 in Tris-Triton buffer.

MPAT: After spotting on the MPAT membrane, samples are probed with the BF9 monoclonal antibody by incubation for 30 min, followed by six washes, processed and visualized as described in Example 3.

A BF9 test assessment in an individual can be translated to an assessment of cancer for the individual, including a score or other identifier that indicates whether an individual has cancer or that indicates a certain likelihood that the individual has cancer or that identifies additional known markers for disease initiation, progression, metastasis or any other characteristic of neoplastic disorders. Similarly, the score or other identifier may indicate a specific type of cancer assessment, such as the assessments of various cancer characteristics described herein, including (but not limited to), determination of whether an individual's cancer is metastasized, determination of the stage of an individual's cancer (such as distinguishing between stage I and stage III cancer), determination of whether an individual's cancer is a malignant tumor or a benign lesion, and determining tumor regression and/or recurrence.

As noted above, the invention includes methods for diagnosing diseases having differential expression of BF9 marker polypeptides. For example, normal, control, or standard values (e.g., that represent typical expression levels of a protein in healthy individuals) for BF9 biomarkers can be established in various assay formats, such as by combining body fluids, tissues, or cell extracts taken from a patient with specific antibodies to a protein under conditions for complex formation. Standard values for complex formation in normal and disease tissues can be established by various methods, such as photometric means. Complex formation, as it is expressed in a test sample, can be compared with the standard values for correlation to disease. Deviation from a normal standard and toward a disease standard can provide parameters for disease diagnosis or prognosis while deviation away from a disease standard and toward a normal standard can be used to evaluate treatment efficacy. Alternately, threshold levels of disease or normal are established.

Platform immunological methods for detecting and measuring complex formation as a measure of the expression of BF9 biomarkers using either specific polyclonal or monoclonal antibodies are known in the art. Examples of such techniques include ELISAs, radio-immunoassays (Ms), flow cytometry (also referred to as fluorescence-activated cell sorting, or FACS), and antibody arrays.

For example, ELISA can be used to detect or quantify BF9 markers. In certain exemplary ELISA methods, an antibody that specifically binds to one such marker may be coated to the well of a suitable container (e.g., a 96 well microliter plate), a patient sample (e.g., a serum sample) can be added to the well and incubated for a period of time, and the presence of said marker in the patient sample can be detected upon binding of an epitope on a BF9 polypeptide in the patient sample to the antibody that is coated to the well. In this instance, a second antibody conjugated to a detectable moiety may optionally be added following the addition of the patient sample to the coated well. ELISA methods such as these may be modified or optimized as desired.

Further, instead of coating the well with the BF9 mAb, BF9 markers may be coated to the well. Thus, in certain ELISA methods, a BF9 polypeptide is coated to the well of a suitable container (e.g., a 96 well microliter plate), a BF9 mAb (which may optionally be conjugated to a detectable moiety such as an enzymatic substrate (horseradish peroxidase or alkaline phosphatase)) is added to the well and incubated for a period of time, and the presence of BF9 marker is detected. An antibody to BF9, whether the BF9 mAb or another, does not have to be conjugated to a detectable moiety; for example, a second antibody (which recognizes the antibody to BF9 or the BF9 mAb disclosed herein) is conjugated to a detectable moeity added to the well.

These assays and their quantitation against purified, labeled standards are well known in the art (Ausubel, supra, unit 10:1-10.6). For example, a two-site, monoclonal-based immunoassay utilizing antibodies reactive to two non-interfering epitopes can be utilized, and competitive binding assay can also be utilized (Pound (1998) Immunochemical Protocols, Humana Press, Totowa N.J.).

For diagnostic applications, an antibody can be labeled with a detectable moiety (interchangeably referred to as a “label” or “detectable substance”), such as to facilitate detection by various imaging methods. Methods for detection of labels include, but are not limited to, fluorescence, light, confocal, and electron microscopy; magnetic resonance imaging and spectroscopy; fluoroscopy, computed tomography and positron emission tomography. Numerous detectable moieties are available for labeling antibodies, including, but not limited to: 1) radioisotopes, such as ³⁶S, ¹⁴C, ¹²⁵I, ³H, and ¹³¹I, 2) fluorescent labels such as rare earth chelates (europium chelates) or fluorescein and its derivatives, rhodamine and its derivatives, dansyl, Lissamine, phycoerythrin and Texas Red, 3) enzyme-substrate labels (e.g., U.S. Pat. Nos. 4,275,149 and 4,318,980).

BF9 can be detected in vivo in an individual patient by introducing into the patient a labeled antibody (or other type of detection agent) specific for the protein marker. For example, an antibody can be labeled with a radioactive marker as described above whose presence and location in an individual can be detected by standard imaging techniques.

The present invention includes the detection of any BF9 marker in the form of polypeptide or polynucleotide, or any combination of 2, 3, 4, or more from the Group 1 consisting of Protein FAM161A (FAM161A, SEQ ID NO: 13); Zinc finger protein 112 (ZNF112, SEQ ID NO: 15); Selenoprotein O (SELENOO, SEQ ID NO: 19); Prame family member 17 (PRAMEF17, SEQ ID NO: 22); Vacuolar protein sorting-associated protein 8 homolog (VPS8, SEQ ID NO: 44).

The present invention includes the detection of any BF9 marker in the form of polypeptide or polynucleotide, or any combination of 2, 3, 4, or more from the Group 2 consisting of Putative uncharacterized protein ASB16-AS1 (ASB16-AS1, SEQ ID NO: 1); Serine/arginine-rich splicing factor 4 (SRSF4, SEQ ID NO: 5); Cytosolic Fe-S cluster assembly factor (NUBP2, SEQ ID NO: 6); Zinc finger protein 214 (ZNF214, SEQ ID NO: 9); Uncharacterized protein C6orf132 (C6orf132, SEQ ID NO: 10); Zinc finger protein 629 (ZNF629, SEQ ID NO: 11); Coiled-coil domain-containing protein 185 (CCDC185, SEQ ID NO: 12); Glycoprotein integral membrane protein 1 (GINM1, SEQ ID NO: 24); Neuroligin-4 X-linked (NLGN4X, SEQ ID NO: 25).

The present invention includes the detection of any BF9 marker in the form of polypeptide or polynucleotide, or any combination of 2, 3, 4, or more from the Group 3 consisting of Spectrin alpha chain, non erythrocytic 1 (SPTAN1, SEQ ID NO: 2); Calcium-binding mitochondrial carrier protein ScaMC-1 (SLC25A24, SEQ ID NO: 14); Zinc finger protein 133 (ZNF133, SEQ ID NO: 16); Protein prune homolog 2 (PRUNE2, SEQ ID NO: 17); Ring finger protein 17 (RNF17, SEQ ID NO: 21); Putative deoxyribonuclease, (TATDN1, SEQ ID NO: 26); Sperm protein associated with the nucleus on the X chromosome (A/C/D) (SPANX, SEQ ID NO: 28); Iron-sulfur protein NUBPL (NUBPL, SEQ ID NO: 35); Myc target protein (MYCT1, SEQ ID NO: 40).

The present invention includes the detection of any BF9 marker in the form of polypeptide or polynucleotide, or any combination of 2, 3, 4, or more from the Group 4consisting of High Mobility Group Protein HMGI-C (HMGA2, SEQ ID NO: 3); Histone H1.2 (HIST1H1C, SEQ ID NO: 4); Glutamine amidotransferase-like class 1 domain-containing protein 3b, mitochondrial (GATD3B, SEQ ID NO: 7); Core histone macro-H2A.2 (H2AFY2, SEQ ID NO: 8); Serine/arginine repetitive matrix protein 3 (SRRM3, SEQ ID NO: 18); Zinc finger protein 536 (ZNF 536, SEQ ID NO: 20); NGFI-A-binding protein 1 (NAB1, SEQ ID NO: 23); Calcineurin B homologous protein 3 (TESC, SEQ ID NO: 27); CUE domain-containing protein 2 (CUEDC2, SEQ ID NO: 29); Nuclease-sensitive element-binding protein 1 (YBX1, SEQ ID NO: 30); Protein S100-A8 (S100A8, SEQ ID NO: 31); Alpha enolase (ENO1, SEQ ID NO: 32); Calmodulin-like protein 5(CALML5, SEQ ID NO: 33); High Mobility Group Protein HMG-I/HMG-Y (HMGA1, SEQ ID NO: 34); Ubiquilin 4 (UBQLN4, SEQ ID NO: 36); Ras-related protein RAB-11B (RAB11B, SEQ ID NO: 37); Vesicle trafficking protein SEC22b (SEC22B, SEQ ID NO: 38); Proline-rich protein 11 (PRR11, SEQ ID NO: 39); Centrin-2 (CETN2, SEQ ID NO: 41); NGFI-A-binding protein 2, (NAB2, SEQ ID NO: 42); Lysine-specific histone demethylase 1A (KDM1A, SEQ ID NO: 43); Polycystic kidney and hepatic disease 1-like protein 1 (PKHD1L1, SEQ ID NO: 45).

The present invention further includes any BF9 marker in the form of polypeptide or polynucleotide, or any combination, of 2, 3, 4, or more from the group consisting of Group 1 and/or Group 2 with any BF9 marker in the form of polypeptide or polynucleotide, or any combination, of 2, 3, 4, or more from the group consisting of Group 3 and/or Group 4 and/or Group 5.

Group 5 comprises the existing markers including but are not limited to Ki-67 (Ki-67), P16INK4a (p16), Estrogen receptor (ER-alfa), Progesterone receptor (PR), c-erbB-2 (HER-2), soluble HER2, Cathepsin D, CA15-3 (CA15-3), CA27.29 (CA27.29), Carcinoma embryonic antigen (CEA), Vimentin (Vimentin), Prostate specific antigen (PSA), Prostatic acid phosphatase (PAP), Kallikrein-2 (KLK-2), p504S (p504S), Tumor Protein p63 (p63), Chromogranin A (CgA), Progastrin releasing peptide type 3(ProGRP), Neuron specific enolase (NSE), Melanocyte lineage-specific antigen (Gp100), MART-1 (MART-1), MAGE-1 (MAGE-1), Calcium binding protein A4/Metastasin 100 (S100A4), Alfa-fetoprotein (AFP), Macrophage inhibitory cytokine (MIC-1), Osteopontin (OSPN), CA19-9 (CA19-9), Mucin-16/ovarian carcinoma antigen CA-125 (CA-125), Leukocyte common antigen (CD45 LCA), CD68 (CD68), Cytokeratins 5, 6 (CK5/6), Cytokeratin 16, 17 and 18 (CK16/17/18), Cytokeratin 17 (CK17), Cytokeratin 19 fragment/CYFRA 21.1, B-cell lymphoma-2 (BCL-2), B-Lymphocyte antigen (CD20), Hematopoietic progenitor CD34 (CD34), Proto-oncogene P53 (p53), Mucin-2 (MUC-2), Mucin-3A (MUC-3), Mucin-4 (MUC-4), Mucin 5AC (MUC-5AC), Mucin-6 (MUC-6), Proliferating cell nuclear antigen (PCNA), Tyrosinase (Tyr), Prostate specific membrane antigen (PSMA-1), Calcium binding protein (S1002), Tissue inhibitor of metalloproteinase (TIMP-1), Squamous cell carcinoma antigen (SCC), Androgen Receptor (ARC), Urokinase plasminogen activator (UPA), Plasminogen activator inhibitor (PAI), Protein uncharacterized ENSP0381381, CA-242, CYFRA21-1.

A description of the use and potential applicability of the markers to the present invention is provided at the following which are incorporated by reference. Duffy M J, Esteva F J, Harbeck N, Hayes D F, Molina R. Tumor markers in breast cancer, In: National Academy of Clinical Biochemistry (NACB) Laboratory Medicine Practice Guidelines “Use of Tumor Markers in testicular, prostate, colorectal, breast and ovarian cancer”, Sturgeon C M, Diamandis E P, Ed., Chapter 5, pp37-49, 2009; Harris L et al., American Society of Clinical Oncology, Update of Recommendations for the Use of Tumor Markers in Breast Cancer, J Clin Oncol 25 (33): 5287-5312, 2007; see also for Goggins M, Koopmann J, Yang D, Canto M I, Hruban R H. National Academy of Clinical Biochemistry (NACB) Laboratory Medicine Practice Guidelines for the Use of Tumor Markers in Pancreatic Ductal Adenocarcinoma. www.nacb.org/tumors, 2005; see also for Brunner N, Duffy M J, Haglund C, Holten-Andersen M, Nielsen H J. Tumor markers in colorectal malignancy, In: National Academy of Clinical Biochemistry (NACB) Laboratory Medicine Practice Guidelines “Use of Tumor Markers in testicular, prostate, colorectal, breast and ovarian cancer”, Sturgeon CM, Diamandis EP, Ed., Chapter 4 , pp27-35, 2009; Locker GY, Hamilton S, Harris J, Jessup JM, Kemeny N, Macdonald JS, Somerfield MR, Hayes DF, Bast RC Jr., ASCO Tumor Panel Expert Panel. ASCO 2006 update of recommendations for the use of tumor markers in gastrointestinal cancer. J Oncol, November 20;24 (33): 5313-27, 2006; see also for Stieber P, Hatz R, Molina R, von Pawel J, Schalhorn A, Schneider J, Yamaguchi K. Tumor markers in lung cancer, In: National Academy of Clinical Biochemistry (NACB) Laboratory Medicine Practice Guidelines “Use of Tumor Markers in testicular, prostate, colorectal, breast and ovarian cancer”, Sturgeon CM, Diamandis EP, Ed., 2006; see also for Chan D W, Bast R C Jr, Shih I-M. Sokoll L, Soletormos G. Tumor markers in ovarian cancer, In: National Academy of Clinical Biochemistry (NACB) Laboratory Medicine Practice Guidelines “Use of Tumor Markers in testicular, prostate, colorectal, breast and ovarian cancer”, Sturgeon C M, Diamandis E P, Ed., Chapter 6, pp51-59, 2009; see also for Lilja H, Semjonow A, Sibley P, Babaian R, Dowell B, Rittenhouse H, Sokoll L. R. Tumor markers in prostate cancer, In: National Academy of Clinical Biochemistry (NACB) Laboratory Medicine Practice Guidelines “Use of Tumor Markers in testicular, prostate, colorectal, breast and ovarian cancer”, Sturgeon C M, Diamandis E P, Ed., Chapter 3, pp15-25, 2009.

BF9 assays are provided that have at least 70% sensitivity at 95% specificity, or at least 70% specificity at 95% sensitivity. In certain embodiments, BF9 assays are provided that have at least 85% sensitivity at 95% specificity, or at least 85% specificity at 95% sensitivity. In further embodiments, BF9 assays are provided that have at least 90% sensitivity or at least 90% specificity or that have at least 95% sensitivity or at least 95% specificity. In yet further embodiments, assays are provided that have at least 70, 75, 80, 85, 90, 91, 92, 93, 94, 95, 96, 97, 98, or 99% (or any other percentage in-between) sensitivity and 70, 75, 80, 85, 90, 91, 92, 93, 94, 95, 96, 97, 98, or 99% (or any other percentage in-between) specificity. In yet further embodiments, BF9 assays are provided that have at least 0.7, 0.75, 0.8, 0.85, 0.9, 0.91, 0.92, 0.93, 0.94, 0.95, 0.96, 0.97, 0.98, or 0.99 (or any other value in-between).

BF9 assay devices for detection of BF9 biomarkers can be provided in the form of kits, such as for use in performing the methods disclosed herein. Furthermore, any kit can contain one or more detectable labels (e.g., detactably labeled reagents such as antibodies), such as a fluorescent moiety, etc. A kit can comprise (a) reagents comprising at least one antibody for detecting BF9 markers, and optionally (b) known markers specific for cancer or a type of cancer of interest.

For immunohistochemistry, a disease tissue sample may be, for example, fresh or frozen or may be embedded in paraffin and fixed with a preservative such as formalin. A fixed or embedded section can be contacted with a labeled primary BF9 antibody and secondary antibody, wherein the antibody is used to detect the expression of BF9 proteins in situ.

Antibodies can be used to detect BF9 polypeptide markers in situ, in vitro, or in a cell lysate or supernatant in order to evaluate the abundance and pattern of expression. Also, antibodies can be used to assess abnormal tissue distribution or abnormal expression during development or progression of a biological condition. Antibodies against BF9 markers are useful for detecting the presence of the proteins in cells or tissues to determine the pattern of expression of the proteins among various tissues in an organism and over the course of the organism's development.

Further, mAb BF9 is used to assess expression in disease states such as in active stages of a disease or in an individual with a predisposition toward disease related to the proteins' function. When a disorder is caused by inappropriate tissue distribution, developmental expression, or level of expression of BF9 markers, an antibody can be prepared against the normal protein. If a disorder is characterized by a specific mutation in a BF9 protein, antibodies specific for the mutant protein can be used to assay for the presence of the specific mutant.

In certain embodiments, the invention provides detection or diagnostic methods of BF9 markers using LC/MS. The differential expression of marker polypeptides detected by mAb BF9 in disease and healthy (or drug-resistant and drug-sensitive, for example) samples can be quantitated using mass spectrometry and ICAT (Isotope Coded Affinity Tag) labeling, which is known in the art. ICAT is an isotope label technique that allows for discrimination between two populations of proteins, such as a healthy and a disease sample. BF9 over-expression or under-expression of BF9, as measured by ICAT, can indicate, for example, the likelihood of having or developing a disease or an associated pathology.

LC/MS spectra can be correlated to disease and normal samples and processed as follows. The raw scans from the LC/MS instrument can be individualized for peak detection and to isolate sequence information using signal/noise reduction software. Filtered peak lists can then be used to detect ‘features’ corresponding to specific BF9 polypeptides from the original sample(s). Features are characterized by their mass/charge ratio, charge, intensity, retention time, isotope pattern, sequence, for example through labeled residue sequencing to determine examples of the BF9 polypeptide sequences herein (SEQ ID NO: 1 to SEQ ID NO: 48) to separate disease from normal.

The BF9 related signal intensity present in both healthy and disease samples can be used to calculate the differential expression, or relative abundance, of the polypeptide. The intensity of a peptide found exclusively in one sample can be used to calculate a theoretical expression ratio for that peptide. Expression ratios can be calculated for each peptide in an assay or experiment.

Natural or synthetic polynucleotides are useful as hybridization probes for determining the presence, level, form, and/or distribution of BF9 nucleic acid expression. Exemplary probes can be used to detect the presence of, or to determine levels of, a specific nucleic acid molecule in cells, tissues, and in organisms. Accordingly, probes corresponding to BF9 related sequences as described herein can be used to assess expression and/or gene copy number in a given patient sample, cell, tissue, or organism, which can be applied to, for example, diagnosis of disorders involving an increase or decrease in corresponding BF9 protein expression relative to normal BF9 protein expression levels.

Nucleic acid test kits for detecting the presence of natural BF9 polynucleotides (e.g., mRNA or genomic DNA) in a biological sample comprise reagents such as a labeled or labelable nucleic acid or agent capable of detecting BF9 polynucleotides in a biological sample and means for comparing the amount of BF9 polynucleotide in the sample with a standard.

Detection of mutations such as deletions, additions, or substitutions of one or more nucleotides in a gene, chromosomal rearrangements (such as inversions or transpositions), and modification of genomic DNA such as aberrant methylation patterns or changes in gene copy number or amplification can be detected at the nucleic acid level by a variety of techniques. For example, genomic DNA or RNA from a patient or group of patients can be analyzed directly or can be amplified (e.g., using PCR) prior to analysis. In certain exemplary embodiments, detection of a mutation involves the use of a probe/primer in a PCR reaction (see, e.g. U.S. Pat. Nos. 4,683,195 and 4,683,202), such as anchor PCR or RACE PCR, or, alternatively, in a ligation chain reaction (LCR) (see, e.g., Landegran et al., Science 241:1077-1080 (1988) and Nakazawa et al., PNAS 91:360-364 (1994)), the latter of which can be particularly useful for detecting point mutations in a gene (see Abravaya et al., Nucleic Acids Res. 23:675-682 (1995)). Exemplary methods such as these can include the steps of collecting a biological sample from a patient, isolating nucleic acid (e.g., genomic, mRNA, or both) from the cells of the sample, contacting the nucleic acid with one or more primers which specifically hybridize to a marker nucleic acid under conditions such that hybridization and amplification of the marker nucleic acid (if present) occurs, and detecting the presence or absence of an amplification product, or defecting the size of the amplification product and comparing the length to a control sample. Deletions and insertions can be detected by a change in size of the amplified product compared to a normal genotype. Point mutations can be identified by hybridizing amplified DNA to normal RNA or antisense DNA sequences, for example.

Alternatively, mutations in BF9 polynucleotides can be identified, for example, by alterations in restriction enzyme digestion patterns as determined by gel electrophoresis. Further, sequence-specific ribozymes (U.S. Pat. No. 5,498,531) can be used to identify the presence of specific mutations by development or loss of a ribozyme cleavage site. Perfectly matched sequences can be distinguished from mismatched sequences by nuclease cleavage digestion assays or by differences in melting temperature.

Sequence changes at specific locations can be assessed by nuclease protection assays such as RNase or chemical cleavage methods. Furthermore, sequence differences between a mutant BF9 gene and a corresponding wild-type gene can be determined by direct DNA sequencing. A variety of automated sequencing procedures can be utilized when performing diagnostic assays (Naeve, C. W., (1995) Biotechniques 19:448), including sequencing by mass spectrometry (e.g., PCT International Publication No. WO 94/16101; Cohen et al., Adv. Chromatogr. 36:127-162 (1996); and Griffin et al., Appl. Biochem. Biotechnol. 38:147-159 (1993)).

Methods for detecting mutations in a BF9 polynucleotide also include methods in which protection from cleavage agents is used to detect mismatched bases in RNA/RNA or RNA/DNA duplexes (Myers et al., Science 230:1242 (1985)); Cotton et al., PNAS 85:4397 (1988); Saleeba et al., Meth. Enzvmol. 217:286-295 (1992)), electrophoretic mobility of mutant and wild type nucleic acid is compared (Orita et al., PNAS 86:2766 (1989); Cotton et al., Maw. Res. 285:125-144 (1993); and Hayashi et al., Genet. Anal. Tech. Appl. 9:73-79 (1992)), and movement of mutant or wild-type fragments in polyacrylamide gels containing a gradient of denaturant is assayed using denaturing gradient gel electrophoresis (DGGE) (Myers et al., Nature 313:495 (1985)). Examples of other techniques for detecting point mutations include selective oligonucleotide hybridization, selective amplification, and selective primer extension.

Natural and synthetic molecules of the invention are also useful for monitoring the effectiveness of modulating agents on the expression or activity of BF9 marker polypeptides, such as in clinical trials or in a treatment regimen. For example, the gene expression pattern of a BF9 natural polynucleotide expression or the presence or amounts of a BF9 marker can serve as a barometer for the continuing effectiveness of treatment. The gene expression pattern can also serve as a marker indicative of a physiological response such as resistance or sensitivity of the cancer cells to the compound. For example, based on monitoring nucleic acid expression, the administration of a compound can be increased or alternative compounds to which the patient has not become resistant can be administered.

In one embodiment, the level of BF9 mRNA is determined either by in situ and by in vitro formats in a biological sample using methods known in the art. Many expression detection methods use isolated RNA. For in vitro methods, any RNA isolation technique that does not select against the isolation of mRNA can be utilized for the purification of RNA from tumor, tissue samples, or tissue cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The mRNA is used in hybridization or amplification assays that include, but are not limited to, Southern or Northern analyses, polymerase chain reaction analyses and probe arrays. One preferred diagnostic method for the detection of mRNA levels involves contacting the mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a marker of the present invention. Other suitable probes for use in the diagnostic assays of the invention are described herein. Hybridization of a BF9 mRNA with the probe indicates that a BF9 marker is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods of several formats having probes linked to a variety of detection systems (such as radioactive or fluorescent probes) for use in detecting the level of BF9 species.

For in situ methods, BF9 mRNA need not be isolated from the tissue or tumor cells prior to detection. In such methods, a cell or tissue sample is prepared/processed using known histological methods. The sample is then immobilized on a support, typically a glass slide, and then contacted with a probe that can hybridize to a given BF9 mRNA.

The invention also includes vectors and host cells containing natural and synthetic BF9 nucleic acid molecules. The term “vector” refers to a vehicle, such as a nucleic acid molecule, which can transport BF9 polynucleotides. When the vector is a nucleic acid molecule, the BF9 polynucleotides are covalently linked to the vector nucleic acid to yield a synthetic polynucleotide. A vector can be, for example, a plasmid, single or double stranded phage, a single or double stranded RNA or DNA viral vector, a mini-locus or artificial chromosome, such as a BAC, PAC, YAC, or MAC. A vector can be maintained in a host cell as an extrachromosomal element such as a plasmid where it replicates and produces additional copies of BF9 polynucleotides or the vector can integrate into the host cell genome and produce additional copies of BF9 polynucleotides when the host cell replicates.

Vectors of the invention include maintenance (cloning vectors) and vectors for expression (expression vectors) of the nucleic acid molecules, for example. Expression vectors can express a portion of, or all of, a protein sequence. Vectors can function in prokaryotic or eukaryotic cells or in both (shuttle vectors). Vectors also include insertion vectors, which integrate a nucleic acid molecule into another nucleic acid molecule, such as into the cellular genome (such as to alter in situ expression of a gene and/or gene product). For example, an endogenous protein-coding sequence can be entirely or partially replaced via homologous recombination with a variant of the protein-coding sequence containing one or more specifically introduced mutations.

Expression vectors can contain cis-acting regulatory regions that are operably linked in the vector to a BF9 polynucleotide such that transcription of the polynucleotide is allowed in a host cell. BF9 polynucleotides can be introduced into the host cell with a separate nucleic acid molecule capable of affecting transcription. The separate nucleic acid molecule may provide, for example, a trans-acting factor interacting with the cis-regulatory control region to allow transcription of the nucleic acid molecules from the vector. Alternatively, a trans-acting factor may be supplied by a host cell. Additionally, a trans-acting factor can be produced from a vector itself.

Regulatory sequences to which BF9 nucleic acid molecules can be operably linked include, for example, promoters for directing mRNA transcription. These include, but are not limited to, the left promoter from T7 bacteriophage promoter, the lac, TRP, and TAC promoters from E. coli, the early and late promoters from SV40, the CMV immediate early promoter, the adenovirus early and late promoters, and retrovirus long-terminal repeats.

In addition to control regions that promote transcription, expression vectors can also include regions that modulate transcription, such as repressor binding sites and enhancers. Examples include the SV40 enhancer, the cytomegalovirus immediate early enhancer, polyoma enhancer, adenovirus enhancers, and retrovirus enhancers.

In addition to containing sites for transcription initiation and control, expression vectors can also contain sequences necessary for transcription termination and, in the transcribed region, a ribosome binding site for translation. Other regulatory control elements for expression include translation, initiation, and termination codons as well as polyadenylation signals. Numerous regulatory sequences useful in expression vectors are well known in the art (e.g., Sambrook et al., Molecular Cloning: A Laboratory Manual. 3rd. ed., Cold Spring Harbor Laboratory Press, Cold Spring Harbor, N.Y. (2001)).

A variety of expression vectors can be used to express a BF9 polynucleotide. Such vectors include chromosomal, episomal, and virus-derived vectors, for example vectors derived from bacterial plasmids, from bacteriophage, from yeast episomes, from yeast chromosomal elements, including yeast artificial chromosomes, from viruses such as baculoviruses, papovaviruses such as SV40, Vaccinia viruses, adenoviruses, poxviruses, pseudorabies viruses, and retroviruses. Vectors may also be derived from combinations of these sources such as those derived from plasmid and bacteriophage genetic elements, e.g. cosmids and phagemids. Appropriate cloning and expression vectors for prokaryotic and eukaryotic hosts are described in Sambrook et al., Molecular Cloning: A Laboratory Manual. 3rd. ed., Cold Spring Harbor Laboratory Press, Cold Spring Harbor, N.Y. (2001). Bacterial cells include, but are not limited to, E. coli, Streptomyces, and Salmonella typhimurium. Eukaryotic cells include, but are not limited to, yeast, insect cells such as Drosophila, animal cells such as COS and CHO cells (e.g., DG44 or CHO-s), and plant cells.

A regulatory sequence can provide constitutive expression in one or more host cells (e.g., tissue specific) or can provide for inducible expression in one or more cell types such as by temperature, nutrient additive, or exogenous factors such as a hormone or other ligand. A variety of vectors providing for constitutive and inducible expression in prokaryotic and eukaryotic hosts are well known in the art.

Recombinant host cells can be prepared by introducing vector constructs, such as described herein, into cells by techniques readily available to a person of ordinary skill in the art. These techniques include, but are not limited to, calcium phosphate transfection, DEAL-dextran-mediated transfection, cationic lipid-mediated transfection, electroporation, transduction, infection, lipofection, microinjection, and other techniques such as those found in Sambrook, et al. (Molecular Cloning: A Laboratory Manual. 3rd. ed., Cold Spring Harbor laboratory Press, Cold Spring Harbor, N.Y. (2001)).

For example, using techniques such as these, a retroviral or other viral vector can be introduced into mammalian cells. Examples of mammalian cells into which a retroviral vector can be introduced include, but are not limited to, primary mammalian cultures or continuous mammalian cultures, COS and CHO cells, NIH3T3, 293 cells (ATCC #CRL 1573), and dendritic cells.

Host cells can contain more than one vector. Thus, different polynucleotide sequences can be introduced on different vectors of the same cell. Similarly, BF9 polynucleotides can be introduced either alone or with other unrelated nucleic acid molecules such as those providing trans-acting factors for expression vectors. When more than one vector is introduced into a cell, the vectors can be introduced independently, co-introduced, or joined to the nucleic acid molecule vector.

Bacteriophage and viral vectors can be introduced into cells as packaged or encapsulated virus by standard procedures for infection and transduction. Viral vectors can be replication-competent or replication-defective. If viral replication is defective, replication can occur in host cells that provide functions that complement the defects.

If secretion of BF9 markers from a host cell is desired, appropriate secretion signals can be incorporated into the vector harboring the expression sequence for that marker. The signal sequence can be endogenous or heterologous to the protein.

Recombinant host cells that express BF9 polypeptides or any variant thereof have a variety of uses. For example, such host cells are useful for producing BF9 variants, which can be further purified to produce desired amounts of the protein or fragments thereof. Thus, host cells containing expression vectors are useful for protein production or for conducting cell-based assays for BF9 polypeptide expression.

### Predictive Medicine

The present invention pertains to the field of predictive medicine in which diagnostic assays, prognostic assays, pharmacokinetics, and pharmacogenomics are used for prognostic (predictive) purposes to identify an asymptomatic patient or patient population or to propose course of for treatment in monitoring a cancer patient undergoing testing or treatment. Accordingly, the present invention includes the process of implementing a protocol for future use of markers for monitoring the progress of a patient or group of patients following a first screening, a first diagnosis, a plurality of additional, subsequent screenings or diagnose or treatment once a BF9 marker is detected. Accordingly, a first test for BF9 is followed by a subsequent test for BF9 or another marker at a future date, including a subsequent screening or analysis of BF9 markers to establish a protocol for diagnosis, including imaging, or treatment including biopsy or other surgical (i.e. resection) or chemical (chemo or immunotherapy) treatment, optimally including a prescribed time interval for future diagnosis or treatment. A preferred protocol for using the BF9 assay or mAb BF9 preferably includes a first screening using the BF9 assay followed by additional testing to monitor expression of BF9 markers, optionally including another marker, at prescribed time intervals to determine progress or stages from an early stage of cancer, the risk of developing cancer beyond the stage assessed at the first screening or predicting the progression of the disease beyond any prior analysis of mAb BF9. Accordingly, the outcome of the BF9 assay on a patient sample may lead a clinician to recommend to the patient to continue the same treatment, or change treatment, to receive confirmatory diagnosis of disease progression through the use of further imaging procedures, or pursue active surveillance at scheduled intervals of time

In the clinical oncology laboratory today single markers (CA15-3, CEA, CYFRA, NSE etc.) are most commonly measured. However diagnostic assays with multiple biomarkers are being developed as they tend to provide higher sensitivity and specificity than a single marker assay. Such assays may comprise measurement of biomarker proteins and autoantibodies (e.g. Videssa Breast; Henderson, 2016), or measurement of methylation of DNA by PCR and hemoglobin by immunoassay (e.g. Cologuard; Imperiale, 2014), or measurement of HE4 (human epididymis protein) and CA125 combined with menopausal status into a numerical score called the risk of malignancy for ovarian cancer (ROMA; Bast, 2012), or microarray analysis of multigene signatures combined to a breast cancer risk score (Oncotype Dx; Paik, 2004). Likewise, it may be found advantageous to adapt the BF9 assay into incorporating the measurement of some BF9 marker species as polypeptides, together with some marker species as polynucleotides, using techniques known in the art for accurate identification of a polypeptide as well as techniques known in the art for accurate identification of a polynucleotide.

The present invention teaches about detection of BF9 markers in tissues, as well as in biological fluids, both in serum and in urine, from a subject in relation to cancer through immunoassay detection. Most conventional diagnostic assays, whether in oncology or other areas, are immunoassays. Indeed immunoassays have significant advantages over alternate methodologies insofar they are serum-based and cost-effective. Additionally, due to their versatility, they come in many test formats, i.e large automated immunoassay platforms and semi-automated smaller formats, and thus specifically tailored to the large and small clinical lab infrastructure settings, respectively. Other immunoassay formats are the commonly known home-based immunoassays, such as urine-based hormone dipstick assays. In a preferred embodiment cancer recurrence may be monitored with patient-friendly home-based assays in urine. At the other end of the spectrum are more sophisticated immunoassays based on nanofludics developments which render immunoassays even more portable and more sensitive, and well suited to point of care testing. In another embodiment of the present invention, rapid point of care cancer testing or therapy or recurrence monitoring is contemplated with the use of hand-held devices.

This invention encompasses all immunoassay formats. Indeed as a way of enabling assay multiplexing, planar solid supports like antibody arrays (Haab, 2006) or bead-based assays, such as but not limited to Luminex technology (Zeh, 2005) can be preferably used to simultaneously measure hundreds of analytes in a biological sample.

It is contemplated in the present invention that data analysis as described herein (Example 10) be improved with the use of further statistical methods, such as but not limited to logistic regression analysis, forest model analysis, and other statistical model that enables the integration of multiple analyte data with patient data (age, gender, stage, ethnicity, smoking status, family and personal history of cancer etc.) to generate a cutoff or a score, based on a robust algorithm, representing a likelihood of having cancer, of disease progression, of being cancer free, or needing further practitioner visit.

In a preferred embodiment the compositions of the invention are applied to small hand-held devices offering rapid immunoassay testing at point of care such as regional community centers or general practitioner's office, preferably in remote areas where patients have limited access to clinical centers and oncologist practices. However a general practitioner, or physician assistant, or skilled nurse may assist a patient in conducting the test of the present invention as well as reading and interpreting test results.

In a further preferred embodiment to ensure the assay of the present invention is made accessible to everyone regardless of the quality of care the subject may access or afford, a software interface is linked to the test to facilitate reading of the results. In some embodiments, a computer-based analysis program is used to translate the raw data generated by the detection assay (e.g. elevation, decrease of a given marker with respect to a reference value) into data of predictive value for a clinician, enabling the latter to make a better clinical assessment of the patient and optimize the care of the subject. The clinician can access the predictive data using any suitable means. Thus, in some preferred embodiments, the present invention provides the benefit that the clinician, or a general practitioner, a physician assistant, a skilled nurse, or the patient is presented with information derived from the raw data in its most useful form, such as for example, in the form of an application translating test results into simple visual instructions as well as recommendations for patient care such as “negative”, “surveillance”, “further testing required”, “follow-up required with doctor”, etc.

1. A method to diagnose breast cancer comprising:
reacting a human serum or urine sample with a non-human monoclonal antibody, wherein the antibody is immunologically reactive with a motif comprising an artificial 12-mer mimotope having at least 67% homology with WHFEFLNIMVNN;
quantifying a total amount of proteins in the serum or urine sample that are immunologically bound to the non-human monoclonal antibody, wherein the serum or urine proteins are comprised of a plurality of Protein FAM161A (FAM161A, SEQ ID NO: 13), Zinc finger protein 112 (ZNF112, SEQ ID NO: 15), Selenoprotein O (SELENOO, SEQ ID NO: 19), Prame family member 17 (PRAMEF17, SEQ ID NO: 22), and Vacuolar protein sorting-associated protein 8 homolog (VPS8, SEQ ID NO: 44), and combinations thereof;
comparing the total quantity of proteins measured in the quantifying step to a reference value; and
diagnosing a presence or absence of breast cancer with at least 70% sensitivity and at least 70% specificity based on a difference between the quantity of total proteins and the reference value.

2. The method of claim 1, wherein the non-human antibody is also immunologically reactive with a motif comprising an artificial 12-mer mimotope having at least 67% homology with SVGDDVAAGVYG and wherein the quantifying step measures a total amount of proteins selected from the group consisting of Putative uncharacterized protein ASB16-AS1 (ASB16-AS1, SEQ ID NO: 1), Serine/arginine-rich splicing factor 4 (SRSF4, SEQ ID NO: 5), Cytosolic Fe-S cluster assembly factor (NUBP2, SEQ ID NO: 6), Zinc finger protein 214 (ZNF214, SEQ ID NO: 9), Uncharacterized protein C6orf132 (C6orf132, SEQ ID NO: 10), Zinc finger protein 629 (ZNF629, SEQ ID NO: 11), Coiled-coil domain-containing protein 185 (CCDC185, SEQ ID NO: 12), Glycoprotein integral membrane protein 1 (GINM1, SEQ ID NO: 24), Neuroligin-4 X-linked (NLGN4X, SEQ ID NO: 25), and combinations thereof.

3. The system of claim 2, wherein the non-human antibody is also immunologically reactive with a motif comprising an artificial 12-mer mimotope having at least 67% homology with RVFETPSMFKER and wherein the quantifying step measures a total amount of protein selected from the group consisting of Spectrin alpha chain, non erythrocytic 1 (SPTAN1, SEQ ID NO: 2), Calcium-binding mitochondrial carrier protein ScaMC-1 (SLC25A24, SEQ ID NO: 14), Zinc finger protein 133 (ZNF133, SEQ ID NO: 16), Protein prune homolog 2 (PRUNE2, SEQ ID NO: 17), Ring finger protein 17 (RNF17, SEQ ID NO: 21), Putative deoxyribonuclease, (TATDN1, SEQ ID NO: 26), Sperm protein associated with the nucleus on the X chromosome (A/C/D) (SPANX, SEQ ID NO: 28), Iron-sulfur protein NUBPL (NUBPL, SEQ ID NO: 35), and Myc target protein (MYCT1, SEQ ID NO: 40), and combinations thereof.

4. The method of claim 1, wherein the human serum or urine sample is comprised of a first sample taken at a first time and a second sample from the patient taken at a second time and the quantifying step is comprised of a comparison of the total quantity of naturally expressed proteins in the serum or urine sample immunologically bound by the non-human antibody at the first time and the serum or urine proteins bound by the non-human antibody at the second time.

5. The method of claim 4, further comprising the step of administering to said patient active surveillance based on the diagnosis.

6. The method of claim 4, further comprising the step of assessing the efficacy of a treatment based on the measurement of the total quantity of the proteins.

7. The method of claim 6, wherein the assessment of the efficacy of the treatment precedes metastasis.

8. A method to diagnose breast cancer comprising:
separating a collection of proteins present in a human serum or urine sample using a reaction mixture comprising a non-human monoclonal antibody that is immunologically reactive with a motif comprising a 12-mer mimotope having at least 67% homology with WHFEFLNIMVNN and is also immunologically reactive with a plurality of proteins selected from the group consisting of Protein FAM161A (FAM161A, SEQ ID NO: 13), Zinc finger protein 112 (ZNF112, SEQ ID NO: 15), Selenoprotein O (SELENOO, SEQ ID NO: 19), Prame family member 17 (PRAMEF17, SEQ ID NO: 22), and Vacuolar protein sorting-associated protein 8 homolog (VPS8, SEQ ID NO: 44), wherein the separating step immunologically binds the plurality of proteins to a solid support comprising the non-human monoclonal antibody immunologically bound to the plurality of naturally expressed proteins present in the human serum or urine sample;
quantifying the collective quantity of the plurality of proteins immunologically bound to the solid support;
comparing the quantified collective amount with a reference value; and
diagnosing a presence or absence of breast cancer with at least 70% sensitivity and at least 70% specificity based on a difference between the collective quantity of the plurality of proteins and the reference value.

9. The method of claim 8, wherein the non-human antibody is also immunologically reactive with a motif comprising a 12-mer mimotope having at least 67% homology with SVGDDVAAGVYG and wherein the plurality of naturally expressed proteins present in the human or urine sample is further comprised of Putative uncharacterized protein ASB16-AS1 (ASB16-AS1, SEQ ID NO: 1), Serine/arginine-rich splicing factor 4 (SRSF4, SEQ ID NO: 5), Cytosolic Fe-S cluster assembly factor (NUBP2, SEQ ID NO: 6), Zinc finger protein 214 (ZNF214, SEQ ID NO: 9), Uncharacterized protein C6orf132 (C6orf132, SEQ ID NO: 10), Zinc finger protein 629 (ZNF629, SEQ ID NO: 11), Coiled-coil domain-containing protein 185 (CCDC185, SEQ ID NO: 12), Glycoprotein integral membrane protein 1 (GINM1, SEQ ID NO: 24), Neuroligin-4 X-linked (NLGN4X, SEQ ID NO: 25), and combinations thereof.

10. The method of claim 8, wherein the non-human antibody is also immunologically reactive with 12-mer mimotope having at least 67% homology with RVFETPSMFKER; and
wherein the plurality of proteins present in the human or urine sample is further comprised of Spectrin alpha chain, non erythrocytic 1 (SPTAN1, SEQ ID NO: 2), Calcium-binding mitochondrial carrier protein ScaMC-1 (SLC25A24, SEQ ID NO: 14), Zinc finger protein 133 (ZNF133, SEQ ID NO: 16), Protein prune homolog 2 (PRUNE2, SEQ ID NO: 17), Ring finger protein 17 (RNF17, SEQ ID NO: 21), Putative deoxyribonuclease, (TATDN1, SEQ ID NO: 26), Sperm protein associated with the nucleus on the X chromosome (A/C/D) (SPANX, SEQ ID NO: 28), Iron-sulfur protein NUBPL (NUBPL, SEQ ID NO: 35), and Myc target protein (MYCT1, SEQ ID NO: 40), and combinations thereof.

11. The method of claim 8, wherein the plurality of naturally expressed proteins present in the human or urine sample is further comprised of High Mobility Group Protein HMGI-C (HMGA2, SEQ ID NO: 3), Histone H1.2 (HIST1H1C, SEQ ID NO: 4), Glutamine amidotransferase-like class 1 domain-containing protein 3b, mitochondrial (GATD3B, SEQ ID NO: 7), Core histone macro-H2A.2 (H2AFY2, SEQ ID NO: 8), Serine/arginine repetitive matrix protein 3 (SRRM3, SEQ ID NO: 18), Zinc finger protein 536 (ZNF 536, SEQ ID NO: 20), NGFI-A-binding protein 1 (NAB1, SEQ ID NO: 23), Calcineurin B homologous protein 3 (TESC, SEQ ID NO: 27), CUE domain-containing protein 2 (CUEDC2, SEQ ID NO: 29), Nuclease-sensitive element-binding protein 1 (YBX1, SEQ IDNO: 30), Protein S100-A8 (S100A8, SEQ IDNO: 31), Alpha enolase (ENO1, SEQ ID NO: 32), Calmodulin-like protein 5 (CALML5, SEQ ID NO: 33), High Mobility Group Protein HMG-I/HMG-Y (HMGA1, SEQ ID NO: 34), Ubiquilin 4(UBQLN4, SEQ ID NO: 36), Ras-related protein RAB-11B (RAB11B, SEQ ID NO: 37), Vesicle trafficking protein SEC22b (SEC22B, SEQ ID NO: 38), Proline-rich protein 11(PRR11, SEQ ID NO: 39), Centrin-2 (CETN2, SEQ ID NO: 41), NGFI-A-binding protein 2, (NAB2, SEQ ID NO: 42), Lysine-specific histone demethylase 1A (KDM1A, SEQ ID NO: 43), Polycystic kidney and hepatic disease 1-like protein 1 (PKHD1L1, SEQ ID NO: 45), and combinations thereof.

12. The method of claim 8, further comprising the step of separately detecting a biomarker selected from the group consisting of Ki-67 (Ki-67), P16INK4a (p16), Estrogen receptor (ER-alfa), Progesterone receptor (PR), c-erbB-2 (HER-2), soluble HER2, Cathepsin D, CA15-3 (CA15-3), CA27.29 (CA27.29), Carcinoma embryonic antigen (CEA), Vimentin (Vimentin), Prostate specific antigen (PSA), Prostatic acid phosphatase (PAP), Kallikrein-2 (KLK-2), p504S (p504S), Tumor Protein p63 (p63), Chromogranin A (CgA), Progastrin releasing peptide type 3 (ProGRP), Neuron specific enolase (NSE), Melanocyte lineage-specific antigen (Gp100), MART-1 (MART-1), MAGE-1 (MAGE-1), Calcium binding protein A4/Metastasin 100 (S100A4), Alfa-fetoprotein (AFP), Macrophage inhibitory cytokine (MIC-1), Osteopontin (OSPN), CA19-9 (CA19-9), Mucin-16/ovarian carcinoma antigen CA-125 (CA-125), Leukocyte common antigen (CD45 LCA), CD68 (CD68), Cytokeratins 5, 6 (CK5/6), Cytokeratin 16, 17 and 18 (CK16/17/18), Cytokeratin 17 (CK17), Cytokeratin 19 fragment/CYFRA 21.1, B-cell lymphoma-2 (BCL-2), B-Lymphocyte antigen (CD20), Hematopoietic progenitor CD34 (CD34), Proto-oncogene P53 (p53), Mucin-2 (MUC-2), Mucin-3A (MUC-3), Mucin-4 (MUC-4), Mucin 5AC (MUC-5AC), Mucin-6 (MUC-6), Proliferating cell nuclear antigen (PCNA), Tyrosinase (Tyr), Prostate specific membrane antigen (PSMA-1), Calcium binding protein (S1002), Tissue inhibitor of metalloproteinase (TIMP-1), Squamous cell carcinoma antigen (SCC), Androgen Receptor (ARC), Urokinase plasminogen activator (UPA), by the non-human antibody at the second Plasminogen activator inhibitor (PAI), and Protein uncharacterized ENSP0381381, CA-242, CYFRA21-1.

13. The method of claim 8, wherein the measurement of the separate biomarker is performed following a first and a second measurement of the plurality of proteins of claim 1 and a comparison of each of the first and second measurement with a reference value, and the measurement of the separate biomarker is correlated to a progression of breast cancer.

14. A method to diagnose breast cancer comprising:
a) reacting a human serum or urine sample with a non-human monoclonal capture antibody fixed on a solid support to immunologically bind a plurality of naturally expressed proteins selected from the group consisting of Protein FAM161A (FAM161A, SEQ ID NO: 13), Zinc finger protein 112 (ZNF112, SEQ ID NO: 15), Selenoprotein O (SELENOO, SEQ ID NO: 19), Prame family member 17 (PRAMEF17, SEQ ID NO: 22), and Vacuolar protein sorting-associated protein 8 homolog (VPS8, SEQ ID NO: 44), and combinations thereof,
wherein the non-human monoclonal capture antibody also immunologically binds a plurality of naturally expressed proteins present in the human or urine sample selected from the group consisting of Putative uncharacterized protein ASB16-AS1 (ASB16-AS1, SEQ ID NO: 1), Serine/arginine-rich splicing factor 4 (SRSF4, SEQ ID NO: 5), Cytosolic Fe-S cluster assembly factor (NUBP2, SEQ ID NO: 6), Zinc finger protein 214 (ZNF214, SEQ ID NO: 9), Uncharacterized protein C6orf132 (C6orf132, SEQ ID NO: 10), Zinc finger protein 629 (ZNF629, SEQ ID NO: 11), Coiled-coil domain-containing protein 185 (CCDC185, SEQ ID NO: 12), Glycoprotein integral membrane protein 1 (GINM1, SEQ ID NO: 24), Neuroligin-4 X-linked (NLGN4X, SEQ ID NO: 25), and combinations thereof; and
wherein the non-human monoclonal capture antibody also immunologically binds a plurality of naturally expressed proteins present in the human or urine sample selected from the group consisting of Spectrin alpha chain, non erythrocytic 1 (SPTAN1, SEQ ID NO: 2), Calcium-binding mitochondrial carrier protein ScaMC-1 (SLC25A24, SEQ ID NO: 14), Zinc finger protein 133 (ZNF133, SEQ ID NO: 16), Protein prune homolog 2 (PRUNE2, SEQ ID NO: 17), Ring finger protein 17 (RNF17, SEQ ID NO: 21), Putative deoxyribonuclease, (TATDN1, SEQ ID NO: 26), Sperm protein associated with the nucleus on the X chromosome (A/C/D) (SPANX, SEQ ID NO: 28), Iron-sulfur protein NUBPL (NUBPL, SEQ ID NO: 35), and Myc target protein (MYCT1, SEQ ID NO: 40), and combinations thereof;
b) labelling a combination of the capture antibody and the immunologically bound proteins with a detection antibody immunologically bound to the combination of the capture antibody and the immunologically bound proteins from the serum or urine sample, wherein the detection antibody is further comprised of a detectable moiety,
c) measuring a quantity of the plurality of immunologically bound proteins using the detection moiety;
d) comparing the measured quantity of the immunologically bound proteins with a reference value; and
e) diagnosing a presence or absence of breast cancer with at least 70% sensitivity and at least 70% specificity based on a difference between the measured quantity of the plurality of immunologically bound proteins and the reference value.

15. A method for detection of breast cancer at an early stage comprising:
a) generating a quantitative measurement of a total amount of a plurality of proteins present in a human serum or urine sample, wherein the naturally expressed human proteins are immunologically bound to a non-human monoclonal antibody that is immunologically reactive with an artificial mimotope having 67% homology with a 12-mer WHFEFLNIMVNN, wherein the non-human monoclonal antibody immunologically binds a plurality of naturally expressed proteins present in the serum or urine sample selected from the group consisting of Protein FAM161A (FAM161A, SEQ ID NO: 13), Zinc finger protein 112 (ZNF112, SEQ ID NO: 15), Selenoprotein O (SELENOO, SEQ ID NO: 19), Prame family member 17 (PRAMEF17, SEQ ID NO: 22), and Vacuolar protein sorting-associated protein 8 homolog (VPS8, SEQ ID NO: 44), and combinations thereof;
b) measuring a total quantity of the immunologically bound naturally expressed proteins bound to the non-human antibody to generate a quantitative value of a summation of the total quantity of immunologically bound proteins;
c) comparing the quantitative value of the total quantity bound proteins with a quantitative reference value; and
d) diagnosing a presence or absence of breast cancer with at least 70% sensitivity and at least 70% specificity based on a difference between the quantity of total proteins and the reference value.

16. A method to diagnose breast cancer comprising:
a) reacting a human serum or urine sample a non-human monoclonal antibody that is immunologically reactive with:
1) an artificial mimotope having at least 67% homology with a 12-mer WHFEFLNIMVNN, and
2) a motif shared by a collection of naturally expressed proteins comprising Protein FAM161A (FAM161A, SEQ ID NO: 13), Zinc finger protein 112 (ZNF112, SEQ ID NO: 15), Selenoprotein O (SELENOO, SEQ ID NO: 19), Prame family member 17 (PRAMEF17, SEQ ID NO: 22), and Vacuolar protein sorting-associated protein 8 homolog (VPS8, SEQ ID NO: 44), and combinations thereof;
b) measuring a collective quantity of proteins having the shared motif and immunologically bound to the non-human monoclonal antibody in the human serum or urine sample;
c) comparing the collective quantity of bound proteins with a reference value; and
d) diagnosing a presence or absence of breast cancer with at least 70% sensitivity and at least 70% specificity based on a difference between the quantity of total bound proteins and the reference value.

17. The method of claim 13, wherein the motif is also shared by Putative uncharacterized protein ASB16-AS1 (ASB16-AS1, SEQ ID NO: 1), Serine/arginine-rich splicing factor 4 (SRSF4, SEQ ID NO: 5), Cytosolic Fe-S cluster assembly factor (NUBP2, SEQ ID NO: 6), Zinc finger protein 214 (ZNF214, SEQ ID NO: 9), Uncharacterized protein C6orf132 (C6orf132, SEQ ID NO: 10), Zinc finger protein 629 (ZNF629, SEQ ID NO: 11), Coiled-coil domain-containing protein 185 (CCDC185, SEQ ID NO: 12), Glycoprotein integral membrane protein 1 (GINM1, SEQ ID NO: 24), Neuroligin-4 X-linked (NLGN4X, SEQ ID NO: 25), and combinations thereof.

18. The method of claim 17, wherein the motif is also shared by Spectrin alpha chain, non erythrocytic 1 (SPTAN1, SEQ ID NO: 2), Calcium-binding mitochondrial carrier protein ScaMC-1 (SLC25A24, SEQ ID NO: 14), Zinc finger protein 133 (ZNF133, SEQ ID NO: 16), Protein prune homolog 2 (PRUNE2, SEQ ID NO: 17), Ring finger protein 17 (RNF17, SEQ ID NO: 21), Putative deoxyribonuclease, (TATDN1, SEQ ID NO: 26), Sperm protein associated with the nucleus on the X chromosome (A/C/D) (SPANX, SEQ ID NO: 28), Iron-sulfur protein NUBPL (NUBPL, SEQ ID NO: 35), and Myc target protein (MYCT1, SEQ ID NO: 40), and combinations thereof.
